(19) United States
(12) Patent Application Publication
(10) Pub. No.: US 2023/0201361 A1
(43) Pub. Date: Jun. 29, 2023

(54) TARGETED AND LOCALIZED IN VIVO DELIVERY OF OLIGONUCLEOTIDES

(71) Applicant: Oncolmmunin, Inc., Gaithersburg, MD (US)

(72) Inventors: Beverly PACKARD, Gaithersburg, MD (US); Akira KOMORIYA, Gaithersburg, MD (US)

(21) Appl. No.: 17/791,846

(22) PCT Filed: Jan. 11, 2021
(86) PCT No.: PCT/US2021/012988

§ 371 Date: Jul. 8, 2022

Related U.S. Application Data
(60) Provisional application No. 62/959,928, filed on Jan. 11, 2020.

(51) Int. Cl. A61K 47/68

(52) U.S. Cl. CPC A61K 47/6807; A61K 47/6849; A61K 47/6851; A61K 47/6889

(57) ABSTRACT

This disclosure provides compositions and methods for the targeted and localized in vivo delivery of oligonucleotides. Compositions containing targeted oligonucleotide-HES conjugates are provided as are methods of making and using the conjugates in therapeutic, diagnostic, and other applications. The oligonucleotide-HES complexes contained in the targeted oligonucleotide-HES conjugates can cross membranes in a receptor-independent manner and can deliver oligonucleotides to complementary sequences in the cytosol of live cells in vivo. The targeted oligonucleotide-HES conjugates have uses that include the targeted and/or localized delivery of antisense oligonucleotides, siRNAs, shRNAs, Dicer substrates, miRNAs, anti-miRNA, and other nucleic acid sequence in a living organism.

### BACKGROUND

This disclosure pertains to the field of oligonucleotide therapeutics. In particular, the disclosure relates targeted conjugates that provide improved targeted and localized in vivo delivery for oligonucleotides including modified oligonucleotides and oligonucleotide mimics, as well as methods of making and using these conjugates.

Oligonucleotides are increasingly recognized for their potential as therapeutic agents against a variety of human diseases. However, a major challenge to the development of therapeutic oligonucleotides is specific and efficient in vivo delivery to the target cells, which is critical to their successful clinical application. Targeted systems can greatly improve the efficiency and specificity of oligonucleotides delivery. However, an effective delivery system must successfully overcome a multitude of biological barriers to enable the oligonucleotides to reach the site of action and access their biological targets. Several targeted delivery strategies based on different platform technologies and different targeting ligands have been developed to achieve these objectives, however, each strategy is associated with limitations that preclude their wide applicability in oligonucleotide therapeutics. Accordingly, there is a need for new targeted oligonucleotide delivery technologies.

### BRIEF SUMMARY

The disclosure relates to Targeted Oligonucleotide-HES conjugate compounds comprising oligonucleotide complexes containing H-type excitonic structures (HES) and methods of making and using these compounds. The disclosure is based in part on the important discovery of the inventors that conjugating an Oligo-HES complex to a targeting moiety results in an increased targeted and localized delivery of the oligonucleotide contained in the targeted oligonucleotide-HES conjugate to targeted nucleic acids in and/or around the cells that express cell surface antigens that are specifically bound by the targeting moiety of the conjugate. The targeted oligonucleotide-HES conjugates dramatically improve the pharmacokinetic properties, selective tissue distribution, and enhance cellular uptake of oligonucleotides in the conjugate in targeted cells and the microenvironment of targeted cells compared to the oligonucleotides alone and in oligonucleotide-HES complexes.

In some embodiments, the disclosure provides
[1] a conjugate comprising a targeting moiety conjugated to an oligonucleotide-HES complex, optionally wherein the oligonucleotide is a therapeutic oligonucleotide;
[2] the conjugate according to [1], wherein the targeting moiety is directly conjugated to the oligonucleotide-HES complex or is conjugated to the oligonucleotide-HES complex through a linker;
[3] the conjugate of [1] or [2], wherein the targeting moiety is conjugated to the oligonucleotide-HES complex through a linker;
[4] the conjugate of any one of [1] to [3] having the structure of formula (I)
$$\text{T-} {\left({\text{L}}_{\text{n}} \text{-} {\left(\text{Oligo-HES}\right)}_{\text{x}}\right)}_{\text{p}}$$
wherein:
   T is a targeting moiety that selectively binds a target of interest;
   L is a linker;
   Oligo-HES is an oligonucleotide complex containing a therapeutic oligonucleotide and an H-type excitonic structure (HES);
   n is 0 or 1;
   x is 1 to 30, 1-20, 1-10, or 1-5; and
   p is 1 to 30, 1-20, 1-10, or 1-5;
[5] the conjugate of any one of [1] to [4] having the structure of formula (II)
$$\text{T-} {\left[{\text{L}}_{\text{n}} \text{-} {\left({\left(\text{Oligo2-SP}\right)}_{\text{m}} \text{-Oligo1-HES}\right)}_{\text{s}}\right]}_{\text{u}}$$
wherein:
   T is a targeting moiety that selectively binds a target of interest;
   L is a linker;
   SP is a linker, optionally wherein SP is 6 to 12 amino acid residue peptide or alkyl chain spacer such as a C6, C10, or C18, linear or branched alkyl;
   Oligo1-HES is an oligonucleotide complex containing oligonucleotide 1 (Oligo1) and an H-type excitonic structure (HES);
   Oligo2 is an oligonucleotide that may be the same or different from Oligo1;
   n is 0 or 1;
   m is 0 or 1;
   s is 1 or 2; and
   u is 1, 2, 3, 4, or 5;
[6] the conjugate of any one of [1] to [5], wherein the oligonucleotide-HES complex comprises a therapeutic oligonucleotide that specifically hybridizes to a nucleic acid sequence in vivo and modulates the level of a protein encoded or regulated by the nucleic acid;
[7] the conjugate of [6], wherein the therapeutic oligonucleotide contains 1, 2, or 3 substitutions, deletions, or insertions, compared to the corresponding reverse complementary strand of the nucleic acid sequence;
[8] the conjugate of any one of [1] to [7], wherein the therapeutic oligonucleotide is from about 8 nucleotides to about 750 nucleotides in length;
[9] the conjugate of any one of [1] to [8], wherein the therapeutic oligonucleotide is 18-25, 18-35, 18-40, or 18-45, 18- 50, 18-60, 18- 70, 18-80, 18- 90, 18-100, 18- 150, or 18-200 nucleotides in length;
[10] the conjugate of any one of [1] to [9], wherein the therapeutic oligonucleotide is single stranded;
[11] the conjugate of any one of [1] to [9], wherein the therapeutic oligonucleotide is double stranded;
[12] The conjugate of [11], wherein the therapeutic oligonucleotide is 36-50, 36-60, 36-70, or 36-100 nucleotides in length;
[13] the conjugate of any one of [1] to [12], wherein the therapeutic oligonucleotide contains one or more modified nucleoside motifs selected from: locked nucleic add (LNA), alpha LNA, 2′-Fluoro (2′F), 2′-O(CH2)2OCH3 (2′-MOE), 2′--deoxy-2′--fluoro-D-arabinonucleic acids (FANA), 2′-OCH3 (2′-O-methyl) (2′OME), PNA, and morpholino;
[14] the conjugate of [13], wherein the modified nucleoside motif is an LNA or alpha LNA in which a methylene (--CH2--)n group bridges the 2′ oxygen atom and the 4′ carbon atom wherein n is 1 or 2;
[15] the conjugate of [13] or [14], wherein the LNA or alpha LNA contains a methyl group at the 5′ position;
[16] the conjugate of any one of [1] to [15], that contains one or more modified internucleoside linkages selected from: phosphorothioate, phosphorodithioate, phosphoramide, 3′-methylene phosphonate, O-methylphosphoroamidiate, PNA and morpholino;
[17] the conjugate of any one of [1] to [16], wherein the therapeutic oligonucleotide contains one or more modified nucleobases selected from C-5 propyne, and 5-methyl C;
[18] the method according to any one of [1] to [17], wherein the therapeutic oligonucleotide of the conjugate specifically hybridizes to a nucleic acid selected from: EGFR, HER2/neu, ErbB3, cMet, p561ck, PDGFR, VEGF, VEGFR, FGF, FGFR, ANG1, ANG2, bFGF,TIE2, protein kinase C-alpha (PKC-alpha), p561ck PKA, TGF-beta, IGFIR, P12, MDM2, BRCA, IGF1, HGF, PDGF, IGFBP2, IGFIR, HIF1 alpha, ferritin, transferrin receptor, TMPRSS2, IRE, HSP27, HSP70, HSP90, MITF, clusterin, PARP1C-fos, C-myc, n-myc, C-raf, B-raf, A1, H-raf, Skp2, K-ras, N-ras, H-ras, farensyltransferase, c-Src, Jun, Fos, Bcr-Abl, c-Kit, EphA2, PDGFB, ARF, NOX1, NF1, STAT3, E6/E7, APC, WNT, beta catenin, GSK3b, PI3k, mTOR, Akt, PDK-1, CDK, Mek1, ERK1, AP-1, P53, Rb, Syk, osteopontin, CD44, MEK, MAPK, NF kappa beta, E cadherin, cyclin D, cyclin E, Bcl2, Bax, BXL-XL, BCL-W, MCL1, ER, MDR, telomerase, telomerase reverse transcriptase, a DNA methyltransferase, a histone deacetlyase (e.g., HDAC1 and HDAC2), an integrin, an IAP, an aurora kinase, a metalloprotease (e.g., MMP2, MMP3 and MMP9), a proteasome, or a metallothionein gene;
[19] the method according to any one of [1] to [17], wherein the therapeutic oligonucleotide of the conjugate specifically hybridizes to a nucleic acid selected from: survivin, HSPB1, EIF4E, PTPN1, RRM2, BCL2, PTEN, Bcr-abl, TLR9, HaRas, Pka-rIA, JNK2, IGF1R, XIAP, TGF-β2, c-myb, PLK1, K-RAS, KSP, PKN3, a Ribonucleotide Reductase (e.g., Ribonucleotide Reductase R1 and Ribonucleotide Reductase R2), a RecQ helicase (e.g., WRN, RecQL1, BLM, RecQL4, RecQ5, and RTS), MEM2 and TLR9;
[20] the conjugate of any one of [1] to [19], wherein the oligonucleotide-HES complex comprises at least 1 fluorophore with an excitation and/or emission from 300-850 nm;
[21] the conjugate of any one of [1] to [20], wherein the oligonucleotide-HES complex comprises 2, 3, 4, or more fluorophores capable of forming one or more HES;
[22] the conjugate of any one of [1] to [21], wherein the oligonucleotide-HES complex comprises 2, 3, 4, or more fluorophores with an excitation and/or emission from 300-850 nm;
[23] the conjugate of any one of [1] to [22], wherein the oligonucleotide-HES complex comprises at least 1 fluorophore selected from a xanthene, an indocarbocyanine, an indodicarbocyanine, and a coumarin;
[24] the conjugate of any one of [1] to [23], wherein the oligonucleotide-HES complex comprises at least 1 fluorophore selected from: carboxyrhodamine 110, carboxytetramethylrhodamine, carboxyrhodamine-X, diethylaminocoumarin and an N-ethyl-N′-[5-(N″-succinimidyloxycarbonyl)pentyl]indocarbocyanine chloride, N-ethyl-N′-[5-(N″-succinimidyloxycarbonyl)pentyl]-3,3,3′,3′-tetramethyl-2′,2′-indodicarbocyanine chloride dye. In further embodiments, the Oligo-HES complex contains a fluorophore selected from the group consisting of: Rhodamine Green™ carboxylic acid, succinimidyl ester or hydrochloride; Rhodamine Green™carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl) sulfonerhodamine or di(succinimidyl, ester); 5-(and-6)-carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethyl ammonium) salt; 5-carboxytetramethylrhodamine; 6-carboxytetramethylrhodamine; 5-(and-6)-carboxytetramethylrhodamine; 5-carboxytetra methylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; Rhodamine Red™-X succinimidyl ester; 6-(tetramethylrhodamine-5-(and-6)-carboxamido)hexanoic acid succinimidyl ester; tetramethylrhodamine-5-isothiocyanate; tetramethylrhodamine-6-isothiocyanate; tetramethylrhodamine-5- (and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6)-isothiocyanate; and a carbocyanine;
[25] the conjugate of any one of [1] to [24], wherein the therapeutic oligonucleotide is selected from: siRNA, shRNA, miRNA, an antagmir, a Dicer substrate, and an antisense;
[26] the conjugate of any one of [1] to [25], wherein the therapeutic oligonucleotide is a siRNA;
[27] the conjugate of any one of [1] to [25], wherein the therapeutic oligonucleotide is a shRNA;
[28] the conjugate of any one of [1] to [25], wherein the therapeutic oligonucleotide is a miRNA or an antagmir (inhibitor of miRNA);
[29] the conjugate of any one of [1] to [25], wherein the therapeutic oligonucleotide is a Dicer substrate;
[30] the conjugate of [29], wherein the therapeutic oligonucleotide contains 2 nucleic complementary nucleic acid strands that are each 18-25, 18-30, 18-35, 18-40, 18-45, or 18-50, nucleotides in length and a 2 nucleotide 3′ overhang;
[31] the conjugate of any one of [1] to [30], wherein the therapeutic oligonucleotide can induce RNA interference (RNAi);
[32] the conjugate of [25], wherein the therapeutic oligonucleotide is a substrate for RNAse H when hybridized to the RNA;
[33] the conjugate of [25], wherein the therapeutic oligonucleotide is a gapmer;
[34] the conjugate of [25], wherein the therapeutic oligonucleotide is not a substrate for RNAse H when hybridized to the RNA;
[35] the conjugate of any one of [1] to [25], wherein the therapeutic oligonucleotide is an antisense oligonucleotide;
[36] the conjugate of [35], wherein the antisense oligonucleotide specifically hybridizes to an RNA of interest;
[37] the conjugate of [35] or [36], wherein the antisense oligonucleotide is DNA or a DNA mimic;
[38] the conjugate of any one of [35] to [37], wherein each nucleoside of the therapeutic antisense oligonucleotide comprises a modified sugar moiety comprising a modification at the 2′-position, a PNA motif, or a morpholino motif;
[39] the conjugate of any one of [35] to [38], wherein, the therapeutic antisense oligonucleotide sequence specifically hybridizes to a target region of an RNA selected from the group consisting of:
   (a) a sequence within 30 nucleotides of the AUG start codon of an mRNA;
   (b) nucleotides 1-10 of a miRNA;
   (c) a sequence in the 5′ untranslated region of an mRNA;
   (d) a sequence in the 3′ untranslated region of an mRNA;
   (e) an intron/exon junction of an mRNA;
   (f) a sequence in a precursor-miRNA (pre-miRNA) or primary-miRNA (pri-miRNA) that when bound by the oligonucleotide blocks miRNA processing; and
   (g) an intron/exon junction and a region 1 to 50 nucleobases 5′ of an intron/exon junction of an RNA;
[40] the conjugate of any one of [35] to [39], wherein hybridization of the oligonucleotide to its target mRNA sterically blocks translation of the coding sequences or modulates expression of the target mRNA, and/or blocks a nucleotide binding protein and thereby modulates target mRNA stability;
[41] The conjugate of any one of [35] to [40], wherein the antisense oligonucleotide-HES complex comprises a plurality of antisense strands, optionally wherein the antisense strands are cross-linked;
[42] the conjugate of [41], wherein the oligonucleotide-HES complex comprises a plurality of antisense oligonucleotides linked in linear with a spacer arm in series 5′ to 3′ and linked with a spacer arm (e.g., 6 to 30 amino acid residue peptide or a linear alkyl (e.g., C6, C10, or C12) or a polyethyloxy-glycol (e.g., triethyloxy-glycol, tetraethyloxy-glycol or hexa-ethyloxy-glycol) to another antisense oligonucleotide linked with 2nd strand 5′ end or 3′ terminal residue;
[43] the conjugate of any one of claims 1 to 42, wherein with linker arm (L or SP) is a linear alkyl of C6, C10, or C12, or a polyethyloxy-glycol (e.g., a triethyloxy-glycol, tetraethyloxy-glycol or hexa-ethyloxy-glycol);
[44] the conjugate of any one of [41] to [43], wherein the Oligo-HES complex comprises a plurality of therapeutic antisense cross-linked with 2 or more antisense strands 5′ to 3′ and 2 or more antisense strands 3′ to 5′ in opposite orientation, Oligo-HES complex comprises a plurality of therapeutic antisense cross-linked with 2 or more antisense strands in 5′ to 3′ with a spacer SP then 3′ to 5′ orientation;
[45] the conjugate of [44], wherein the antisense strands have 2 or more of the same complementary sequence and/or 2 or more different complementary sequences;
[46] the conjugate of any one of [1] to [45], which comprises a linear or branched linker;
[47] the conjugate of any one of [1] to [46], which comprises a peptide linker or SP spacer of 6 to 30 amino acid residues in length;
[48] the conjugate of [46] or [47], wherein the linker is a linear peptide of 6 to 30 amino acid residues in length;
[49] the conjugate of any one of [46] to [48], wherein the linker is cleavable;
[50] the conjugate of [49], wherein the linker has an amino acid sequence comprising a protease cleavage site containing P1-P1′ residues and having a looped conformation;
[51] the conjugate of [49] or [50], wherein the cleavable linker comprises an amino acid sequence that is a substrate for at least one protease;
[52] the conjugate of any one [49] to [51], wherein the cleavable linker comprises an amino acid sequence that is a substrate for at least one protease that is active in a diseased tissue;
[53] the conjugate of any one of [49] to [52], wherein the cleavable linker comprises an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease;
[54] the conjugate of any one of [49] to [53], wherein the cleavable linker comprises an amino acid sequence that is a substrate for at least one protease selected from:
   (a) MMP9;
   (b) MMP14;
   (c) MMP1, MMP2, MMP3, MMP7, MMP8, MMP10, MMP11, MMP12, MMP13, MMP15, MMP16, MMP17, MMP19, MMP20, MMP23, MMP24, MMP26, and MMP27;
   (d) a serine protease (e.g., MT-SP1, uPA, and TMPRSS2);
   (e) a cysteine protease;
   (f) a metalloprotease including (a)-(c);
   (g) an aspartyl protease; and
   (h) a threonine protease;
[55] the conjugate of [54], wherein the cleavable linker comprises an amino acid sequence that is a substrate for MMP9 or MMP14;
[56] the conjugate of [54] or [55], wherein the cleavable linker comprises an amino acid sequence that is a substrate for at least one enzyme of the immune complement system such as u-plasminogen activator, tissue plasminogen activator, trypsin, and plasmin;
[57] the conjugate of any one of [49] to [56], wherein the cleavable linker comprises an amino acid sequence that is a substrate for a protease known or reported to be colocalized with the target of the conjugate;
[58] the conjugate of [49], wherein the linker is cleavable under intracellular conditions (e.g., conditions within a lysosome or endosome or caveolae);
[59] the conjugate of [49], wherein the linker is pH-sensitive;
[60] the conjugate of [49], wherein the linker is cleavable under reducing conditions;
[61] the conjugate of conjugate of any of [44] to [56], wherein the linker is a malonate linker;
[62] the conjugate of any of [46] to [48], wherein the linker is non-cleavable;
[63] the conjugate of any one of [46] to [62], wherein the linker has an H-dimer forming fluorophore (e.g., a linker comprising an H-dimer forming fluorophore conjugated at the amino and/or carboxyl terminal residues);
[64] the conjugate of any one of [46] to [63], wherein the linker has an H-dimer forming fluorophore conjugated at the amino and carboxyl terminal residues;
[65] the conjugate of any one of [46] to [64], wherein the linker has a sulfhydryl or amino functional group at the amino and carboxyl terminus of the peptide;
[66] the conjugate of any one of [1] to [65], wherein the targeting moiety of the conjugate is an aptamer, avimer, a receptor-binding ligand, a nucleic acid, a biotin-avidin binding pair, a peptide, protein a carbohydrate, lipid, vitamin, a component of a microorganism, a hormone, a receptor ligand (including Fc fusion proteins containing the same), an antibody, an antigen binding portion of an antibody, an alternative binding scaffold, or any derivative thereof;
[67] the conjugate of any one of [1] to [66], wherein the targeting moiety is an antibody, an antigen binding portion of an antibody (e.g., a Fab, and a scFv), or a single-domain antibody;
[68] the conjugate of any one of [1] to [67], wherein the targeting moiety is an antibody, a humanized antibody, an antigen binding fragment of an antibody, a single chain antibody, a bi-specific antibody, a synthetic antibody, or a pegylated antibody;
[69] the conjugate of any one of [1] to [68], wherein the targeting moiety is an antibody;
[70] the conjugate of [69], wherein the targeting moiety is a monospecific, bispecific or multispecific antibody and/or a monovalent, bivalent, or multivalent antibody;
[71] the conjugate of [69] or [70], wherein the targeting moiety is an IgG1, IgG2, or IgG4 antibody;
[72] the conjugate of any one of [69] to [70], wherein the targeting moiety is a therapeutic antibody.
[73] the conjugate of [72], wherein the antibody is selected from: trastuzumab (HER2/neu), pertuzumab (HER2/neu), panitumumab (EGFR), nimotuzumab (EGFR), zalutumumab (EGFR), cetuximab (EGFR), (HER3), onartuzumab (c-MET), patritumab, clivatuzumab (MUC1), sofituzumab (MUC16), edrecolomab, (EPCAM), adecatumumab (EPCAM), anetumab (MSLN), huDS6 (CA6), lifastuzumab (NAPI2B), sacituzumab (TROP2), PR1A3, humanized PR1A3 (CEA), humanized Ab 2-3 (CEA), IMAB362/claudiximab (Claudin18.2), AMG595 (EGFRvIII), ABT806 (EGFRvIII), sibrotuzumab (FAP), DS-8895a variant 1 (EphA2), DS-8895a variant 2 (EphA2), anti-EphA2 (EphA2), MEDI-547 (EphA2), narnatumab (RON), RG7841 (LY6E), farletuzumab (FRA /folate receptor alpha), mirvetuximab (FRA), J591 variant 1 (PSMA), J591 variant 2 (PSMA), rovalpituzumab (DLL3), PF-06647020 (PTK7), anti-PTK7 (PTK7), ladiratuzumab (LIV1), cirmtuzumab (ROR1), rituximab (CD20), ibritumomab tiuxetan (CD52), alemtuzumab (CD33), Gemtuzumab ozogamicin (CD33), CT-011 (PD1), tositumomab (CD20), ipilimumab (CTLA4), tremelimumab (CP-675,206)(CTLA4), nivolumab (PD1), and pembrolizumab (PD1), durvalumab (PDL1) anti-MAGE-A3, anti-NY-ESO-1, anti-ACE2, anti-hyaluronidase, or anti-neuraminidase;
[74] the conjugate of any one of [1] to [68], wherein the targeting moiety is an antigen binding fragment of an antibody, a single chain antibody, a single-domain antibody, or a bi-specific antibody;
[75] the conjugate of any one of [1] to [66], wherein the targeting moiety is an alternative binding scaffold selected from: an affibody, nanobody, anticalin, fynomer, DARPin, Tetranectin, Transbody, AdNectin, Affilin, Microbody, peptide aptamer, alterase, plastic antibody, phylomer, stradobody, maxibody, evibody, Z domain, D domain, armadillo repeat protein, Kunitz domain, avimer, atrimer, probody, immunobody, triomab, troybody, pepbody, vaccibody, UniBody, Affimer, or a DuoBody;
[76] the conjugate of any one of [1] to [75], wherein the targeting moiety specifically binds a cell surface antigen on or near a cell or tissue of interest such as, a diseased cell, a cancer cell, an immune cell, an infected cell, or an infectious agent;
[77] the conjugate of any one of [1] to [76], wherein the targeting moiety specifically binds a cell surface antigen(s) derived, from or determined to be expressed on, a specific subject’s cancer (e.g., tumor) such as a neoantigen;
[78] the conjugate of any one of [1] to [77], wherein the targeting moiety specifically binds a cell surface antigen that does not internalize the conjugate upon binding;
[79] the conjugate of any one of [1] to [75], wherein the targeting moiety specifically binds a cell surface antigen that internalizes the conjugate upon binding;
[80] the conjugate of any one of [1] to [79], wherein the targeting moiety specifically binds a tumor cell surface antigen;
[81] the conjugate of any one of [1] to [80], wherein the targeting moiety specifically binds a tumor cell surface antigen on a leukemic cell, lymphoma cell, pancreatic cancer cell, breast cancer cell, melanoma cell, lung cancer cell, head and neck cancer cell, ovarian cancer cell, bladder cancer cell, colon cancer cell, kidney cancer cell, liver cancer cell, prostate cancer cell, bone cancer cell, or brain cancer cell including a glioblastoma cell; or any lymphoma, myeloma, blastoma, sarcoma, leukemia or carcinoma cell;
[82] the conjugate of any one of [1] to [81], wherein the targeting moiety specifically binds a cell surface antigen selected from: CD5, CD19, CD20, CD25, CD37, CD30, CD33, CD45, CD204, CD206, CD301, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Ley, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, HER2/neu, MAGE A3, P53 nonmutant, NY-ESO-1, MelanA/MART1, Ras mutant, gp100, P53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PDL, VTCN1, and VISTA;
[83] the conjugate of any one of [1] to [81], wherein the targeting moiety specifically binds a cell surface antigen selected from: HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15;
[84] the conjugate of any one of [1] to [83], wherein the targeting moiety specifically binds a tumor microenvironment cell surface antigen (including certain membrane anchored proteases);
[85] the conjugate of any one of [1] to [84], wherein the targeting moiety specifically binds a cell surface antigen expressed on endothelial cells or macrophages (e.g., VEGFR, TIE1, and TIE2), or tumor stromal cells such as cancer-associated fibroblasts (CAFs), and tumor infiltrating T cells and other leukocytes, and myeloid cells including mast cells, eosinophils, and tumor-associated macrophages;
[86] the conjugate of any one of [1] to [85], wherein the targeting moiety specifically binds a cell surface antigen on an immune cell;
[87] the conjugate of [86], wherein the targeting moiety specifically binds a cell surface antigen on an immune cell of lymphoid or myeloid origin such as a T cell, a B cell, an NK cell, an NKT cell, or a dendritic cell;
[88] the conjugate of [86] or [87], wherein the targeting moiety specifically binds a cell surface antigen on an antigen presenting cell;
[89] The conjugate of [88], wherein the targeting moiety specifically binds an antigen selected from: OX40L, 4-1BBL, MARCO, DC-SIGN, Dectin1, Dectin2, DEC-205, CLEC5A, CLEC9A, CLEC10A, CLEC12A, CD1A, CD16A, CD32A, CD32B, CD36, CD40, CD47, CD64, CD204, CD206, HVEM, PDL1, mannose scavenger receptor1, and BDCA2;
[90] the conjugate of [86] or [87], wherein the targeting moiety specifically binds a cell surface antigen on an immune cell that is not an antigen presenting cell;
[91] the conjugate of any one of [1] to [90], wherein the targeting moiety binds the target (e.g., cell surface antigen) of interest with an equilibrium dissociation constant (Kd) in a range of 0.5 × 10⁻¹⁰ to 10 × 10⁻⁶ as determined using BIACORE® analysis;
[92] a method for modulating a nucleic acid or protein level in a cell, said method comprising contacting the cell with a therapeutically effective amount of the conjugate of any one of [1] to [91], wherein the oligonucleotide-HES complex comprises:
   (a) a Targeting moiety that binds to a cell surface antigen on the cell or a nearby cell; and
   (b) an oligonucleotide that specifically hybridizes to the nucleic acid and modulates the level of the nucleic acid and/or protein encoded or regulated by the nucleic acid;
[93] the method of [92], wherein the targeting moiety of the T-Oligo-HES conjugate specifically binds a cell surface antigen on the contacted cell and the cell expresses a cell surface protease that cleaves a cleavable linker of the T-Oligo-HES conjugate to release an Oligo-HES complex.
[94] the method of [92] or [93], wherein the modulated nucleic acid or protein is in a diseased cell, an infected cell, an infectious agent, or an immune cell;
[95] the method of [94], wherein the modulated nucleic acid or protein is in a diseased cell;
[96] the method of [95], wherein the modulated nucleic acid or protein is in a cancer cell;
[97] the method of [96], wherein the cancer cell is a hematologic cancer cell or a solid tumor cancer cell;
[98] the method of [97], wherein the cancer cell is a leukemic cell, pancreatic cancer cell, breast cancer cell, melanoma cell, lung cancer cell, head and neck cancer cell, ovarian cancer cell, bladder cancer cell, colon cancer cell, kidney cancer cell, liver cancer cell, prostate cancer cell, bone cancer cell, or brain cancer cell including a glioblastoma cell; or any lymphoma, myeloma, blastoma, sarcoma, leukemia or carcinoma cell;
[99] the method of [94], wherein the modulated nucleic acid or protein is in an infected or infectious cell;
[100] the method of [99], wherein the modulated nucleic acid or protein is in an infected cell or an infectious agent;
[101] the method of [99] or [100], wherein the cell is infected with: HIV, HTLV-1, Zika Virus, Dengue Virus, Influenza Virus, Ebola Virus, Marburg Virus, Crimean Congo Hemorrhagic Fever Virus, Lassa Fever Virus, Variola Virus, SARS Virus, Rift Valley Fever Virus, TB, Anthrax, Botulism, Tularemia, Plague, Brucellosis, glanders, Mellioidosis, Q fever, or an Alpha virus such as Chikungunya virus, Sindbis virus, Semliki Forest virus, the western, eastern and Venezuelan equine encephalitis viruses, the Ross River virus, COVID, or Influenza;
[102] the method of [94], wherein the modulated nucleic acid or protein is in an immune cell;
[103] the method of [102], wherein the immune cell is a cell of lymphoid of myeloid origin such as a T cell, a B cell, an NK cell, an NKT cell, or a dendritic cell;
[104] the method of any one of [92] to [103], wherein the therapeutic oligonucleotide is selected from: siRNA, shRNA, miRNA, antagmir, a Dicer substrate, and an antisense;
[105] the method of any one of [92] to [104], wherein the conjugate comprises a cleavable linker containing an amino acid sequence that is a substrate for at least one protease;
[106] the method of [105], wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease;
[107] the method of any one of [92] to [106], wherein the targeting moiety of the conjugate is an antibody, an antigen binding portion of an antibody (e.g., a Fab, and a scFv), or a single-domain antibody;
[108] a method for modulating a nucleic acid or protein level in a subject, said method comprising administering a therapeutically effective amount of the conjugate of any one of [1] to [91] to a subject in need thereof, and wherein the conjugate comprises:
   (a) a targeting moiety that binds to a cell surface antigen on or near a cell in which the nucleic acid or protein is to be modulated; and
   (b) an oligonucleotide that specifically hybridizes to the nucleic acid and modulates the level of the nucleic acid and/or protein encoded or regulated by the nucleic acid
[109] the method of [108], wherein the nucleic acid or protein is characterized by
   (a) overexpression or underexpression of the nucleic acid in the subject, or
   (b) overexpression or underexpression of the protein encoded by the nucleic acid in the subject;
[110] the method of [108] or [109], wherein the modulated nucleic acid or protein is in a diseased cell, an infected cell, an infectious agent, or an immune cell;
[111] the method of [108], wherein the modulated nucleic acid or protein is in a diseased cell;
[112] the method of [110], wherein the modulated nucleic acid or protein is in a cancer cell;
[113] the method of [112], wherein the cancer cell is a hematologic cancer cell or a solid tumor cancer cell;
[114] the method of [113], wherein the cancer cell is a leukemic cell, pancreatic cancer cell, breast cancer cell, melanoma cell, lung cancer cell, head and neck cancer cell, ovarian cancer cell, bladder cancer cell, colon cancer cell, kidney cancer cell, liver cancer cell, prostate cancer cell, bone cancer cell, or brain cancer cell including a glioblastoma cell; or any lymphoma, myeloma, blastoma, sarcoma, leukemia or carcinoma cell;
[115] the method of [110], wherein the modulated nucleic acid or protein is in an infected cell or an infectious agent;
[116] the method of [115, where the modulated nucleic acid or protein is in an infectious agent;
[117] the method of [115], wherein the infected cell is an immune cell of lymphoid or myeloid origin;
[118] the method of [110], wherein the modulated nucleic acid or protein is in an immune cell;
[119] the method of [118], wherein the immune cell is a lymphoid or a myeloid cell such as, a T cell, a B cell, an NK cell, an NKT cell, or a dendritic cell;
[120] the method of any one of [108] to [119], wherein the therapeutic oligonucleotide is selected from: a siRNA, a shRNA, a miRNA, an antagmir, a Dicer substrate, and an antisense;
[121] the method of any one of [108] to [120], wherein the conjugate comprises a cleavable linker containing an amino acid sequence that is a substrate for at least one protease;
122] the method of [121], wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease;
[123] the method of any one of [108] to [122], wherein the conjugate targeting moiety is an antibody, an antigen binding portion of an antibody (e.g., a Fab, and a scFv), or a single-domain antibody;
[124] a method for treating a disease or disorder in a subject, said method comprising administering to a subject in need thereof, a therapeutically effective amount of the conjugate of any one of [1] to [91], wherein the oligonucleotide specifically hybridizes to a nucleic acid sequence in vivo and modulates the level of a protein encoded or regulated by the nucleic acid; and wherein the disease or disorder is characterized by:
   (a) overexpression or underexpression of the target a nucleic acid in the subject, or
   (b) overexpression or underexpression of a protein encoded by the target nucleic in the subject;
[125] the method of [124], wherein the conjugate comprises a targeting moiety that binds to a cell surface antigen on or near a cell in which the nucleic acid or protein is to be modulated;
[126] the method of [124] or [125], wherein the disease or disorder is a proliferative disease or disorder such as cancer, a disease or disorder of the immune system, an inflammatory disease or disorder, an infectious disease, a neurological disease or disorder, a disease or disorder of the cardiovascular system, a metabolic disease or disorder, a disease or disorder of the skeletal system, or a disease or disorder of the skin or eyes;
[127] the method of any one of [124] to [126], wherein the disease or disorder is cancer, an inflammatory disease or disorder, or a disease or disorder of the immune system, an infectious disease, or a neurological or disease or disorder such as a neurodegenerative disease or disorder;
[128] the method of any one of [124] to [127], wherein the disease or disorder is cancer;
[129] the method of [128], wherein the cancer is a hematologic cancer or a solid tumor cancer;
[130] the method of [129], wherein the cancer is leukemia, pancreatic cancer, breast cancer, melanoma, lung cancer, head and neck cancer, ovarian cancer, bladder cancer, colon cancer, kidney cancer, liver cancer, prostate cancer, bone cancer, or brain cancer including a glioblastoma; or any lymphoma, myeloma, blastoma, sarcoma, leukemia or carcinoma;
[131] the method of [124], wherein the disease or disorder is an inflammatory disease or disorder, a disease or disorder of the immune system, or an infectious disease;
[132] the method of [131], wherein the disease is an inflammatory disease or disorder, or an autoimmune disease or disorder (e.g., rheumatoid arthritis);
[133] the method of [132], wherein the disease or disorder is inflammation;
[134] the method of [133], wherein the disease or disorder is an infectious disease;
[135] the method of [134], wherein the infectious disease is HIV, HTLV-1, Zika, Dengue, Influenza, Ebola, Marburg, Crimean Congo Hemorrhagic Fever, Lassa Fever Virus, Variola, SARS, Rift Valley Fever, TB, Anthrax, Botulism, Tularemia, Plague, Brucellosis, glanders, Mellioidosis, Q fever, or infected with an Alpha virus such as Chikungunya virus, Sindbis virus, Semliki Forest virus, the western, eastern and Venezuelan equine encephalitis viruses, the Ross River virus, or COVID;
[136] the method of [126], wherein the disease or disorder is a neurological disease or disorder;
[137] the method of [136], wherein the neurological disease or disorder is a neurodegenerative disease such as familial and sporadic amyotrophic lateral sclerosis (FALS and ALS, respectively), familial and sporadic Parkinson’s disease, Huntington’s disease (Huntington’s chorea), familial and sporadic Alzheimer’s disease, Spinal Muscular Atrophy (SMA), multiple sclerosis, diffuse cerebral cortical atrophy, dementia, or Pick disease;
[138] the method of any one of [124] to [137], wherein the therapeutic oligonucleotide of the conjugate is selected from: a siRNA, a shRNA, a miRNA, an antagmir, a Dicer substrate, and an antisense;
[139] the method of any one of [124] to [138], wherein the conjugate comprises a cleavable linker containing an amino acid sequence that is a substrate for at least one protease;
[140] the method of [139], wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease;
[141] the method of any one of [124] to [140], wherein the targeting moiety of the conjugate is an antibody, an antigen binding portion of an antibody (e.g., a Fab, and a scFv), or a single-domain antibody;
[142] the method of any one of [124] to [141], wherein the targeting moiety of the conjugate is an antibody or antigen binding fragment of an antibody;
[143] the method of [142], wherein the targeting moiety is an antibody or nanobody;
[144] the method of [143], wherein the antibody is an IgG1, IgG2, or IgG4 antibody;
[145] the method of [143] or [144], wherein the antibody is a therapeutic antibody;
[146] the method of any one of [124] to [145], wherein the conjugate is administered simultaneously, sequentially or separately with one or more other therapeutic drugs;
[147] a method of treating cancer in a subject, said method comprising administering a therapeutically effective amount of the conjugate of any one of [1] to [91] to a subject in need thereof], wherein the oligonucleotide specifically hybridizes to a nucleic acid in the cancer cell or tissue and modulates the level of the nucleic acid and/or protein encoded or regulated by the nucleic acid, and wherein the nucleic acid or protein is characterized by:
   (a) overexpression or underexpression of the nucleic acid in the cancer cell, tissue, and/or subject, or
   (b) overexpression or underexpression of the protein encoded by the nucleic acid in the cancer cell, tissue, and/or subject;
[148] the method of [147], wherein the targeting moiety of the conjugate specifically binds to a cell surface antigen on or near the cancer cell;
[149] the method of [147] or [148], wherein the targeting moiety of the conjugate specifically binds to a cell surface antigen on the cancer cell;
[150] the method of any one of [147] to [149], wherein the targeting moiety of the conjugate specifically binds to a cell surface antigen on a cell near the cancer cell (e.g., a cell in the tumor microenvironment such as a stromal cell, cancer associated fibroblast, immune cell, blood or lymphatic vascular cell, endothelial cell, adipose cell, or neuroendocrine cell);
[151] the method of any one of [147] to [150], wherein the cancer is a solid tumor;
[152] the method of any one of [147] to [151], wherein the cancer is leukemia, pancreatic cancer, breast cancer, melanoma, lung cancer, head and neck cancer, ovarian cancer, bladder cancer, colon cancer, kidney cancer, liver cancer, prostate cancer, bone cancer, or brain cancer including a glioblastoma; or any lymphoma, myeloma, blastoma, sarcoma, leukemia or carcinoma;
[153] the method of [152], wherein the cancer is a hematologic cancer;
[154] the method of [153], wherein the hematologic cancer is a leukemia or lymphoma;
[155] the method of any one of claims 147 to 154, wherein the targeting moiety of the conjugate specifically binds a cell surface antigen selected from: CD5, CD19, CD20, CD25, CD37, CD30, CD33, CD45, CD204, CD206, CD301, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Ley, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, HIV GP120, HIV GP160, EGFRvIII, HER2/neu, MAGE A3, P53 nonmutant, NY-ESO-1, MelanA/MART1, Ras mutant, gp100, P53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PDL, VTCN1, and VISTA;
[156] the method of any one of [147] to [154], wherein the targeting moiety of the conjugate specifically binds a cell surface antigen selected from: DLL3, fibroblast activation protein α (FAPα), NG2 (Neuroglial Antigen-2), platelet-derived growth factor receptor-β (PDGFR-β), PD1, CD163, KIR, HMGB1, VEGFR3, LYVE1, CD31, CD34, P1GF, and VEGF;
[157] the method of any one of [147] to [154], wherein the targeting moiety of the conjugate specifically binds a cell surface antigen selected from: HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, Clorf186, and LRRC15;
[158] the method of any one of [147] to [156], wherein the therapeutic oligonucleotide of the conjugate is selected from: a siRNA, a shRNA, a miRNA, an antagmir, a Dicer substrate, and an antisense;
[159] The method according to any one of [147] to [157], wherein the therapeutic oligonucleotide of the conjugate specifically hybridizes to a nucleic acid selected from:
   EGFR, HER2/neu, ErbB3, cMet, p56lck, PDGFR, VEGF, VEGFR, FGF, FGFR, ANG1, ANG2, bFGF,TIE2, protein kinase C-alpha (PKC-alpha), p56lck PKA, TGF-beta, IGFIR, P12, MDM2, BRCA, IGF1, HGF, PDGF, IGFBP2, IGFlR, HIF1 alpha, ferritin, transferrin receptor, TMPRSS2, IRE, HSP27, HSP70, HSP90, MITF, clusterin, PARPlC-fos, C-myc, n-myc, C-raf, B-raf, A1, H-raf, Skp2, K-ras, N-ras, H-ras, farensyltransferase, c-Src, Jun, Fos, Bcr-Abl, c-Kit, EphA2, PDGFB, ARF, NOX1, NF1, STAT3, E6/E7, APC, WNT, beta catenin, GSK3b, PI3k, mTOR, Akt, PDK-1, CDK, Mek1, ERK1, AP-1, P53, Rb, Syk, osteopontin, CD44, MEK, MAPK, NF kappa beta, E cadherin, cyclin D, cyclin E, Bcl2, Bax, BXL-XL, BCL-W, MCL1, ER, MDR, telomerase, telomerase reverse transcriptase, a DNA methyltransferase, a histone deacetlyase (e.g., HDAC1 and HDAC2), an integrin, an IAP, an aurora kinase, a metalloprotease (e.g., MMP2, MMP3 and MMP9), a proteasome, or a metallothionein gene;
[160] the method according to any one [147] to [157], wherein the therapeutic oligonucleotide of the conjugate specifically hybridizes to a nucleic acid selected from:
   survivin, HSPB1, EIF4E, PTPN1, RRM2, BCL2, PTEN, Bcr-abl, TLR9, HaRas, Pka-rIA, JNK2, IGF1R, XIAP, TGF-β2, c-myb, PLK1, K-ras, KSP, PKN3, a Ribonucleotide Reductase (e.g., Ribonucleotide Reductase R1 and Ribonucleotide Reductase R2), a RecQ helicase (e.g., WRN, RecQL1, BLM, RecQL4, RecQ5, and RTS), MEM2 and TLR9;
[161] the method of any one of [147] to [159], wherein the conjugate comprises a cleavable linker containing an amino acid sequence that is a substrate for at least one protease.
[162] the method of [160], wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease;
[163] the method of any one of [147] to [161], wherein the conjugate targeting moiety is an antibody, an antigen binding portion of an antibody (e.g., a Fab, and a scFv), or a single-domain antibody;
[164] the method of any one of [147] to [162], wherein the targeting moiety of the conjugate is an antibody or antigen binding fragment of an antibody;
[165] the method of [163], wherein the targeting moiety is an antibody;
[166] the method of [164], wherein the antibody is an IgG1, IgG2, or IgG4 antibody;
[167] the method of [164] or [165], wherein the antibody is a therapeutic antibody;
[168] the method of [166], wherein the antibody is selected from: trastuzumab (HER2/neu), pertuzumab (HER2/neu), panitumumab (EGFR), nimotuzumab (EGFR), zalutumumab (EGFR), cetuximab (EGFR), (HER3), onartuzumab (c-MET), patritumab, clivatuzumab (MUC1), sofituzumab (MUC16), edrecolomab, (EPCAM), adecatumumab (EPCAM), anetumab (MSLN), huDS6 (CA6), lifastuzumab (NAPI2B), sacituzumab (TROP2), PR1A3, humanized PR1A3 (CEA), humanized Ab 2-3 (CEA), IMAB362/claudiximab (Claudin18.2), AMG595 (EGFRvIII), ABT806 (EGFRvIII), sibrotuzumab (FAP), DS-8895a variant 1 (EphA2), DS-8895a variant 2 (EphA2), anti-EphA2 (EphA2), MEDI-547 (EphA2), narnatumab (RON), RG7841 (LY6E), farletuzumab (FRA /folate receptor alpha), mirvetuximab (FRA), J591 variant 1 (PSMA), J591 variant 2 (PSMA), rovalpituzumab (DLL3), PF-06647020 (PTK7), anti-PTK7 (PTK7), ladiratuzumab (LIV1), cirmtuzumab (ROR1), rituximab (CD20), ibritumomab tiuxetan (CD52), alemtuzumab (CD33), Gemtuzumab ozogamicin (CD33), CT-011 (PD1), tositumomab (CD20), ipilimumab (CTLA4), tremelimumab (CP-675,206)(CTLA4), nivolumab (PD1), and pembrolizumab (PD1), durvalumab (PDL1) anti-MAGE-A3, and anti-NY-ESO-1;
[169] the method of any one of [146] to [167], wherein the conjugate is administered simultaneously, sequentially or separately with one or more other anticancer drugs.
[170] a conjugate according to any one of [1] to [91] for use in medicine;
[171] a conjugate as defined to any one [1] to [91] for use in treatment of a disease or disorder in a subject;
[172] the conjugate according to any one [1] to [91] for use in treating a disease or disorder selected from: an infectious disease, cancer, a proliferative disease or disorder, a neurological disease or disorder, and inflammatory disease or disorder, a disease or disorder of the immune system, a disease or disorder of the cardiovascular system, a metabolic disease or disorder, a disease or disorder of the skeletal system, and a disease or disorder of the skin or eyes;
[173] a conjugate according to any one of [1] to [91] for use in modulating a target nucleic acid or protein subject; treating a disease or disorder characterized by overexpression or underexpression of a nucleic acid in a subject, treating a disease or disorder characterized by overexpression or underexpression of a protein in a subject; treating a disease or disorder characterized by aberrant nucleic acid or protein expression in a subj ect.

Still other features and advantages of the compositions and methods described herein will become more apparent from the following detailed description when read in conjunction with the accompanying drawings.

### BRIEF DESCRIPTION OF THE DRAWINGS/FIGURES

FIG. 1 depicts the exemplary synthesis of a T (antibody)-oligo-HES conjugate. Structures of some click chemistry reagents are simplified for illustrative purposes. Linkers are covalently bound to an antibody through either the antibody’s amino groups or its sulfhydryls, the latter following reduction with agents such as dithiothreitol or TCEP. For labeling amino groups, functional groups such as N-hydroxy-succinimidyl esters or other leaving groups can be used. For labeling the antibody’s sulfhydryls, functional groups such as maleimides or 3-arylpropiolonitriles can be used. The linker arm typically ranges between six and fifty atoms. The functional group on the distal end of the linker, i.e., the end furthest from the antibody, can be either an azide or an alkyne, the latter preferably a carbon-carbon triple bond in a strained conformation such as a cyclooctyne ring with sp2 orbitals on both sides of the triple bond, e.g., a dibenzocyclooctyne. Click chemistry in which an alkyne or an azide, respectively, can then be used to conjugate the linker on the antibody to a peptide containing a conformation-dependent cleavage site which will serve as a cleavage site for a protease on a cell surface. The peptide will be conjugated on the side distal to the antibody linkage to an oligonucleotide, either a single strand antisense or a double strand siRNA. Both the peptide containing the conformation-dependent cleavage site and the oligonucleotide will each be previously derivatized with two fluorophores such that they form an HES, specifically, a peptide HES and an oligonucleotide HES. Structures of some click chemistry reagents are simplified for illustrative purposes.

FIG. 2. Exemplary derivatization of an antibody covalently labeled with a linker arm. An IgG2a was first reduced with TCEP and then a linker with 25 atoms was added. The linker which terminated with a dibenzocyclooctyne group was then conjugated with an azide-bearing fluorophore. After a 1 hour reaction time, the solution was passed over a gel filtration column with the conjugate eluting immediately after the void volume. The peak at 280 nm is due to the antibody and the peak at 641 indicates the covalent attachment of the fluorophore. The complete synthesis of T (e.g., antibody)-oligo-HES conjugates can occur in multiple steps such as, for example, (a) by adding the linker first and then sequentially adding a peptide containing a conformation-dependent cleavage site and an oligonucleotide or (b) by adding the linker already conjugated to the peptide containing the conformation-dependent cleavage site and oligonucleotide directly to the antibody. Each step can be checked by using complementary functional chemical groups containing reporter groups such as fluorophores.

FIG. 3. Specificity of conformation-dependent cleavage site. An eighteen amino acid peptide containing the amino acid sequence of PLGIA (SEQ ID NO:77) and covalently labeled with the same fluorophore near each end (giving rise to an HES structure) is recognized by matrix metalloprotease -9 (MMP-9) and is cleaved between the LG and the IA. MMP-19 is added in a pH 7.5 buffer in which the PLGIA (SEQ ID NO:77) peptide is at 2 uM. Cleavage of the peptide gives rise to an increase in fluorescence intensity. This specificity is compared with a control peptide, i.e., an HES-bearing peptide of the same length, with the same labeling, but that does not contain the conformation-dependent cleavage site. The fluorescence of the latter does not increase upon addition of the MMP indicating specificity of the PLGIA (SEQ ID NO:77) sequence for MMP-19.

FIGS. 4A-4B. Fragmentation/cleavage of a PLGIA (SEQ ID NO:77) peptide containing a conformation-dependent cleavage site. The retention time of the HES-PLGIA (SEQ ID NO:77) peptide containing a conformation-dependent cleavage site was determined by HPLC where the retention time on a C18 column was determined under reverse phase conditions, i.e., loading in an aqueous buffer and eluting in an acetonitrile buffer, to be 38 minutes (FIG. 4A). After exposure to MMP-9, the major peaks were at ca. 30 and 31 minutes with the almost complete disappearance of the 38 minute peak (FIG. 4B), consistent with the cleavage as indicated in FIG. 3.

FIG. 5. Formation of an antibody linked to a peptide containing a conformation-dependent cleavage site. Rituximab, a monoclonal antibody which recognizes CD20 on B-lymphocytes, was conjugated to a linker and a peptide containing a conformation-dependent cleavage site. The peak at 280 nm indicates the presence of the antibody. The conformation-dependent specificity of the peptide cleavage site which is due to the presence of the HES is indicated by the more intense peak at 520 nm relative to that at 552 nm. If the two fluorophores which form the intramolecular H-dimer were not present, then the peak at 552 would be higher than that at 520. Thus, the conformational specificity of the peptide is maintained after covalent bond formation with the linker.

FIG. 6. Recognition of rituximab-oligo-HES conjugate by B cells. Raji cells, a CD20+ B lymphocyte cell line, was exposed to rituximab labeled with a linker arm and a peptide containing a conformation-dependent cleavage site at 4° C. After washing and addition of a viability dye, the cells were examined by flow cytometry. Cells exposed to the modified rituximab conjugate recognized the antibody and bound it with no effect on the cells′ viability. Thus, modification of the antibody by the chemistry of addition by click chemistry did not diminish the recognition function of the monoclonal antibody.

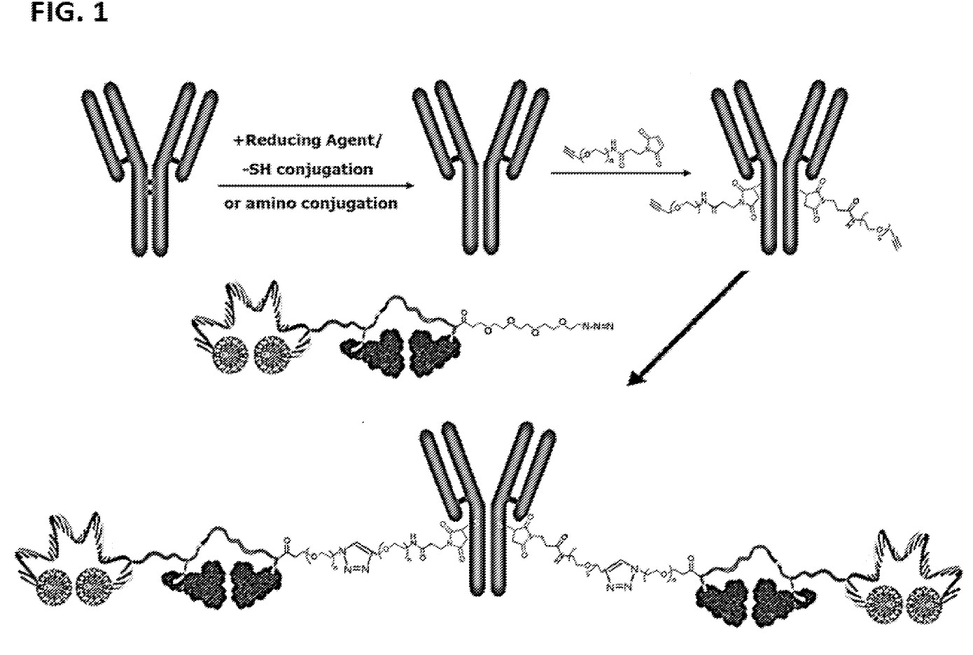

### DETAILED DESCRIPTION

This disclosure provides compositions and methods for the targeted and localized in vivo delivery of oligonucleotides. Compositions containing targeted oligonucleotide-HES conjugates are provided as are methods of making and using the conjugates in therapeutic, diagnostic, and other applications. The oligonucleotide-HES complexes contained in the targeted oligonucleotide-HES conjugates can cross membranes in a receptor-independent manner and can deliver oligonucleotides that complementary sequences into the cytosol of live cells in vivo. The targeted oligonucleotide-HES conjugates have uses that include the targeted and/or localized delivery of antisense oligonucleotides, siRNAs, shRNAs, Dicer substrates, miRNAs, anti-miRNA, and other nucleic acid sequence in a living organism.

### Definitions

The meaning of certain terms recited herein are provide below or elsewhere in the disclosure:

The terms “nucleic acid” or “oligonucleotide” refer to at least two nucleotides covalently linked together. A nucleic acid oligonucleotide provided herein is preferably single-stranded or double-stranded and generally contains phosphodiester bonds, although in some cases, as outlined below, nucleic acid/oligonucleotide analogs are included that have alternate backbones, comprising, for example, phosphoramide (see, e.g., Beaucage et al., Tetrahedron 49(10): 1925 (1993)) and references therein; Letsinger, J. Org. Chem. 35:3800 (1970); Sprinzl et al., Eur. J. Biochem. 81: 579 (1977); Letsinger et al., Nucl. Acids Res. 14:3587 (1986); Sawai et al., Chem. Lett. 805 (1984); Letsinger et al., J. Am. Chem. Soc. 1 10:4470 (1988); and Pauwels et al., Chemica Scripta 26: 1419 (1986), the entire contents of each of which is herein incorporated by reference in its entirety), phosphorathioate (Mag et al., Nucleic Acids Res. 19:1437 (1991); and U.S. Pat. No 5,644,048, the entire contents of each of which is herein incorporated by reference in its entirety), phosphorodithioate (Briu et al., J. Am. Chem. Soc. 111:2321 (1989)), O-methylphosphoroamidiate linkages (see, e.g., Eckstein, Oligonucleotides and Analogues: A Practical Approach, Oxford University Press), and peptide nucleic acid backbones and linkages (see, e.g., Egholm, J. Am. Chem. Soc. 1 14: 1895 (1992); Meier et al., Chem. Int. Ed. Engl. 31: 1008 (1992); Nielsen, Nature 365:566 (1993); Carlsson et al., Nature 380:207 (1996), the entire contents of each of which is herein incorporated by reference in its entirety). Other analog nucleic acids/oligonucleotides include those with positive backbones (see, e.g., Dempcy et al., Proc. Natl, Acad. Sci USA 92:6097 (1995), the entire contents of each of which is herein incorporated by reference in its entirety); non-ionic backbones (see, e.g., U.S. Pat. Nos. 5,386,023, 5,637,684, 5,602,240, 5,216,141, and 4,469,863; Angew, Chem. Intl, Ed. English 30:423 (1991); Letsinger et al., J. Am. Chem. Soc. 110:4470 (1988); Letsinger et al., Nucleoside & Nucleotide 13: 1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Chaturvedi et al., Tetrahedron Lett. 37:743 (1996), the entire contents of each of which is herein incorporated by reference in its entirety), and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y.S. Sanghui and P. Dan Cook. Nucleic acids/oligonucleotides containing one or more carbocyclic sugars are also included within the definition of nucleic acids/oligonucleotides (see, e.g., Jenkins et al., Chem. Soc. Rev. pp 169-176 (1995), the entire contents of each of which is herein incorporated by reference in its entirety). Several nucleic acid/oligonucleotide analogs are described in Rawls, C & E News Jun. 2, 1997, page 35, which is herein incorporated by reference in its entirety). These modifications of the ribose-phosphate backbone may be done for example, to facilitate the addition of additional moieties such as labels, or to increase the stability and half-life of such molecules in physiological environments. Nucleic acid/oligonucleotide backbones of oligonucleotides provided herein range from about 5 nucleotides to about 750 nucleotides. Preferred nucleic acid/oligonucleotides in the T-Oligo-HES conjugates provided herein range from about 5 nucleotides to about 500 nucleotides, and preferably from about 10 nucleotides to about 100 nucleotides in length. As used herein, the term “about” or “approximately” when used in conjunction with a number refers to any number within 0.25%, 0.5%, 1%, 5% or 10%) of the referenced number.

The oligonucleotides in the Oligo-HES complexes of the T-Oligo-HES conjugates are polymeric structures of nucleoside and/or nucleotide monomers capable of specifically hybridizing to at least a region of a nucleic acid target. As indicated above, HES-oligonucleotides include, but are not limited to, compounds comprising naturally occurring bases, sugars and intersugar (backbone) linkages, non-naturally occurring modified monomers, or portions thereof (e.g., oligonucleotide analogs or mimetics) which function similarly to their naturally occurring counterpart, and combinations of these naturally occurring and non-naturally occurring monomers. As used herein, the term “modified” or “modification” includes any substitution and/or any change from a starting or natural oligomeric compound, such as an oligonucleotide. Modifications to oligonucleotides encompass substitutions or changes to internucleoside linkages, sugar moieties, or base moieties, such as those described herein and those otherwise known in the art.

The term “antisense” as used herein, refers to an oligonucleotide sequence, written in the 5′ to 3 ′ direction, comprises the reverse complement of the corresponding region of a target nucleic acid and/or that is able to specifically hybridize to the target nucleic acid under physiological conditions. Thus, in some embodiments, the term antisense refers to an oligonucleotide that comprises the reverse complement of the corresponding region of a small noncoding RNA, untranslated mRNA and/or genomic DNA sequence. In particular embodiments, an antisense oligonucleotide in a T-Oligo-HES conjugate provided herein, once hybridized to a nucleic acid target, is able to induce or trigger a reduction in target gene expression, target gene levels, or levels of the protein encoded by the target nucleic acid.

“Complementary,” as used herein, refers to the capacity for pairing between a monomeric component of an oligonucleotide and a nucleotide in a targeted nucleic acid (e.g., DNA, mRNA, and a non-coding RNA such as, a raiRNA). For example, if a nucleotide at a certain position of an oligonucleotide is capable of hydrogen bonding with a nucleotide at the same position of a DNA/RNA molecule, then the oligonucleotide and DNA/RNA are considered to be complementary at that position.

In the context of this application, “hybridization” means the pairing of an oligonucleotide with a complementary nucleic acid sequence. Such pairing typically involves hydrogen bonding, which may be Watson-Crick, Hoogsteen or reversed Hoogsteen hydrogen bonding, between complementary nucleoside or nucleotide bases (nucleobases) of an oligonucleotide and a target nucleic acid sequence (e.g., wherein the oligonucleotide comprises the reverse complementary nucleotide sequence of the corresponding region of the target nucleic acid). In particular embodiments, an oligonucleotide specifically hybridizes to a target nucleic acid. The terms “specifically hybridizes” and “specifically hybridizable” are used interchangeably herein to indicate a sufficient degree of complementarity such that stable and specific binding occurs between the oligonucleotide and the target nucleic acid (i.e., DNA or RNA). It is understood that an oligonucleotide need not be 100% complementary to its target nucleic acid sequence to be specifically hybridizable. In particular embodiments, an oligonucleotide is considered to be specifically hybridizable when binding of the oligonucleotide to a target nucleic acid sequence interferes with the normal function of the target nucleic acid and results in a loss or altered utility or expression therefrom. In preferred embodiments, there is a sufficient degree of complementarity between the oligonucleotide and target nucleic acid to avoid or minimize non-specific binding of the oligonucleotide to undesired non-target sequences under the conditions in which specific binding is desired (e.g., under physiological conditions in the case of in vivo assays or therapeutic treatment, and in the case of in vitro assays, under conditions in which the assays are performed). It is well within the level of skill of scientists in the oligonucleotide field to routinely determine when conditions are optimal for specific hybridization to a target nucleic acid with minimal non-specific hybridization events. Thus, in some embodiments, oligonucleotides in the T-Oligo-HES conjugate includes 1, 2, or 3 base substitutions compared to the corresponding complementary sequence of a region of a target DNA or RNA sequence to which it specifically hybridizes. In some embodiments, the location of a non- complementary nucleobase is at the 5′ end or 3′ end of an antisense oligonucleotide. In additional embodiments, a non-complementary nucleobase is located at an internal position in the oligonucleotide. When two or more non-complementary nucleobases are present in an oligonucleotide, they may be contiguous (i.e., linked), non-contiguous, or both. In some embodiments, the oligonucleotides in the complexes provided herein have at least 85%, at least 90%, or at least 95% sequence identity to a target region within the target nucleic acid. In other embodiments, oligonucleotides have 100% sequence identity to a polynucleotide sequence within a target nucleic acid. Percent identity is calculated according to the number of bases that are identical to the corresponding nucleic acid sequence to which the oligonucleotide being compared. This identity may be over the entire length of the oligomeric compound (i.e., oligonucleotide), or in a portion of the oligonucleotide (e.g., nucleobases 1-20 of a 27-mer may be compared to a 20-mer to determine percent identity of the oligonucleotide to the oligonucleotide). Percent identity between an oligonucleotide and a target nucleic acid can routinely be determined using alignment programs and BLAST programs (basic local alignment search tools) known in the art (see, e.g., Altschul et al., J. Mol. Biol., 215:403-410 (1990); Zhang and Madden, Genome Res., 7:649-656 (1997)).

As used herein, the terms “target nucleic acid” and “nucleic acid encoding a target” are used to encompass any nucleic acid capable of being targeted including, without limitation, DNA encoding a given molecular target (i.e., a protein or polypeptide), RNA (including miRNA, pre-mRNA and mRNA) transcribed from such DNA, and also cDNA derived from such RNA. Exemplary DNA functions to be interfered with include replication, transcription and translation. The overall effect of such interference with target nucleic acid function is modulation of the expression of the target molecule. As used herein, “modulation” means a quantitative change, either an increase (stimulation) or a decrease (inhibition), for example in the expression of a gene. The inhibition of gene expression through reduction in RNA levels is a preferred form of modulation.

As used herein, the terms “pharmaceutically acceptable,” or “physiologically tolerable” and grammatical variations thereof, as they refer to compositions, carriers, diluents and reagents, are used interchangeably and represent that the materials are capable of administration to or upon a subject (e.g., a mammal such as a mouse, rat, rabbit, or a primate such as a human), without the production of therapeutically prohibitive undesirable physiological effects such as nausea, dizziness, gastric upset and the like.

As used herein, a “pharmaceutical composition comprising an antisense oligonucleotide” refers to a composition comprising an T-Oligo-HES conjugate and a pharmaceutically acceptable diluent. By way of example, a suitable pharmaceutically acceptable diluent is phosphate-buffered saline.

A “stabilizing modification” or “stabilizing motif means providing enhanced stability, in the presence of nucleases, relative to that provided by 2 -deoxy nucleosides linked by phosphodiester internucleoside linkages. Thus, such modifications provide “enhanced nuclease stability” to oligonucleotides. Stabilizing modifications include at least stabilizing nucleosides and stabilizing internucleoside linkage groups.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The terms “administering” and “administration” as used herein, refer to adding a chemical such as an oligonucleotide to a subject in vivo or ex vivo. Thus, administering encompasses both the addition of an HES-oligonucleotide directly to a subject and also contacting cells with HES-oligonucleotide compositions and then introducing the contacted cells into a subject. In one embodiment, cells removed from a subject are contacted with an HES-oligonucleotide and the contacted cells are then re-introduced to the subject. The term “contacting” refers to adding a chemical such as an oligonucleotide to an in vivo organism such as a mammal, plant, bacterium, or virus. For mammals, common routes of contacting include peroral (through the mouth), topical (skin), transmucosal (nasal, buccal/sublingual, vaginal, ocular and rectal), inhalation (lungs), intramuscular (muscle) and intravenous (vein). For bacteria and viruses contact may be delivery inside a cell or tissue of a host organism.

“Treating” or “treatment” includes the administration of an HES-oligonucleotide to prevent or delay the onset of the symptoms, complications, or biochemical indicia of a disease, condition, or disorder, alleviating the symptoms or arresting or inhibiting further development of the disease, condition, or disorder. Treatment can be prophylactic (to prevent or delay the onset of the disease, or to prevent the manifestation of clinical or subclinical symptoms thereof) or therapeutic suppression or alleviation of symptoms after the manifestation of the disease, condition, or disorder. Treatment can be with an HES- oligonucleotide complex containing composition alone, or in combination with 1, 2, 3 or more additional therapeutic agents.

The term “therapeutically effective amount” refers to an amount of an HES-oligonucleotide complex (“therapeutic agent”) or other drug effective to achieve a desired therapeutic result and/or to “treat” a disease or disorder in a subject. The term “therapeutically effective amount” may also refer to an amount required to produce a slowing of disease progression, an increase in survival time, and/or an improvement in one or more indicators of disease or the progression of a disease in a subject suffering from the disease. For example, in the case of cancer, a therapeutically effective amount an HES-oligonucleotide complex may: reduce angiogenesis and neovascularization; reduce the number of cancer cells, a therapeutically effective amount an HES-oligonucleotide complex may reduce tumor size, inhibit (i.e., slow or stop) cancer cell infiltration into peripheral organs, inhibit (i.e., slow or stop) tumor metastasis, inhibit or slow tumor growth or tumor incidence, stimulate immune responses against cancer cells and/or relieve one or more symptoms associated with the cancer. In the case of an infectious disease, a therapeutically effective amount an HES-oligonucleotide complex may be associated with a reduced number of the infectious agent (e.g., viral load) and/or in amelioration of one or more symptoms or conditions associated with infection caused by the infectious agent. A “therapeutically effective amount” also may refer to an amount effective, at dosages and for periods of time necessary, to achieve a desired therapeutic result. A therapeutically effective amount of an HES-oligonucleotide complex may vary according to factors such as, the disease state, age, sex, and weight of the subject, and the ability of the HES-oligonucleotide complex to elicit a desired response in the subject. A therapeutically effective amount is also one in which any toxic or detrimental effects of the HES-oligonucleotide complex are outweighed by the therapeutically beneficial effects.

“Therapeutic index” means the ratio of the dose of an HES-oligonucleotide complex which produces an undesired effect to the dose which causes desired effects. In the context of the present disclosure, an HES-oligonucleotide complex exhibits an “improved therapeutic index” when activity is retained, but undesired effects are reduced or absent. For example, an HES-oligonucleotide complex having an improved therapeutic index retains the ability to inhibit miRNA activity without resulting in undesired effects such as immunostimulatory activity, or, at least, without resulting in undesired effects to a degree that would prohibit administration of the complex.

As used herein a “therapeutic oligonucleotide” refers to an oligonucleotide capable of achieving a desired therapeutic result and/or to “treat” a disease or disorder in a subject or ex vivo when administered at sufficient doses. Such desirable results include for example, a slowing of disease progression, an increase in survival time, and/or an improvement in one or more indicators of disease, disease progression, or disease related conditions in a subject suffering from the disease. Exemplary therapeutic oligonucleotides include a siRNA, a shRNA, a Dicer substrate (e.g., dsRNA), a miRNA, an anti-miRNA, an antisense, a decoy, an aptamer and a plasmid capable of expressing a siRNA, a miRNA, a ribozyme, an antisense oligonucleotide, or a protein coding sequence. Oligonucleotides such as probes and primers that are not able to achieve a desired therapeutic result are not considered therapeutic oligonucleotides for the purpose of this disclosure. On average, less than 1% of mRNA is a suitable target for antisense oligonucleotides. Numerous antisense oligonucleotides suitable for incorporation to the HES-oligonucleotide complexes contained in the provided T-Oligo-HES conjugates are described herein or otherwise known in the art. Likewise, suitable therapeutic oligonucleotides can routinely be designed using guidelines, algorithms and programs known in the art (see, e.g., Aartsma-Rus et al., Mol. Ther. 17(3):548-553 (2009) and Reynolds et al., Nat. Biotech. 22(3):326-330 (2004), and Zhang et al., Nucleic Acids Res. 31 e72 (2003), the contents of each of which is herein incorporated by reference in its entirety). Suitable therapeutic oligonucleotides can likewise routinely be designed using commercially available programs (e.g., MysiRNA-Designer, AsiDesigner (Bioinformatics Research Center, KRIBB), siRNA Target Finder (Ambion), Block-iT RNAi Designer (Invitrogen), Gene specific siRNA selector (The Wistar Institute), siRNA Target Finder (GeneScript), siDESIGN Center (Dharmacon), SiRNA at Whitehead, siRNA Design (IDT), D: T7 RNAi Oligo Designer (Dudek P and Picard D.), sfold-software, and RNAstructure 4.5); programs available over the internet such as, human splicing finder software (e.g., at “.umd.be/HSF/”) and Targetfmder (available at “bioit.org.cn/ao/targetfinder”); and commercial providers (e.g., Gene Tools, LLC). In certain instances, an oligonucleotide, Oligonucleotide-HES, HES-Oligonoucleotide, or Oligo-HES, and a therapeutic oligonucleotide may be used interchangeably herein unless the context clearly dictates otherwise.

As used herein, a “therarapeutic antibody” in the context of a T-Oligo-HES conjugate provided herein refers to a targeting moiety that is an antibody that binds to a therapeutic target molecule and is expected to result in alleviation, or a decrease in the progression, of a disease in vivo.

The terms “specifically binds” or “specific affinity” in the context of a therapeutic antibody or other targeting moiety mean that a targeting moiety such as an antibody or antigen binding antibody fragment, reacts or associates more frequently, more rapidly, with greater duration, with greater affinity, or with some combination of the above to the epitope, protein, or target molecule than with alternative substances, including proteins unrelated to the target epitope. Because of the sequence identity between homologous proteins in different species, specific affinity can, in several embodiments, include a binding agent that recognizes a protein or target in more than one species. Likewise, because of homology within certain regions of polypeptide sequences of different proteins, the term “specific affinity” or “specifically binds” can include a binding agent that recognizes more than one protein or target. It is understood that, in certain embodiments, a targeting moiety that specifically binds a first target may or may not specifically bind a second target. As such, “specific affinity” does not necessarily require (although it can include) exclusive binding, e.g., binding to a single target. Thus, a targeting moiety may, in certain embodiments, specifically bind more than one target. In certain embodiments, multiple targets may be bound by the same targeting moiety. In some embodiments, the targeting moiety (e.g., a therapeutic antibody) binds a target (e.g., cell surface antigen) with an equilibrium dissociation constant (Kd) in a range of 0.5 × 10⁻¹⁰ to 10 × 10⁻⁶ as determined using BIACORE® analysis

The term “infectious agent” as used herein refers to agents that cause an infectious disease. Infectious agents belong to four main groups: viruses, bacteria, fungi, and parasites. Said infectious agents can be extracellular or intracellular (e.g., an infected cell).

The term “infectious disease” as used herein refers to diseases caused by infectious agents such as bacteria, viruses, parasites or fungi. Infectious diseases can be spread, directly or indirectly (through a vector and/or reservoir), from one organism (e.g., human) to another.

### T-Oligo-HES Connjugates

The provided T-oligonucleotide-HES (T-Oligo-HES) conjugates comprise a targeting moiety (T) conjugated to an oligonucleotide-HES complex (Oligo-HES), wherein the targeting moiety is optionally conjugated to the Oligo-HES complex through a linker. In particular embodiments, the targeting moiety is conjugated to the Oligo-HES complex through a linker. In further particular embodiments, the targeting moiety is conjugated to the Oligo-HES complex through a cleavable linker. In particular embodiments, an oligonucleotide contained in the T-Oligo-HES conjugate is a therapeutic oligonucleotide. In further particular embodiments, the conjugate contains a therapeutic oligonucleotide selected from an siRNA, shRNA, miRNA, an antagmir, a dicer substrate, an antisense oligonucleotide, and an expression cassette (e.g., plasmid) capable of expressing an siRNA, a miRNA, a ribozyme or an antisense oligonucleotide.

In particular embodimnets, the T-Oligo-HES conjugate comprises a targeting moiety conjugated to the Oligo-HES complex through a linker and the conjugate comprises a therapeutic oligonucleotide. In further particular embodiments, the T-Oligo-HES conjugate comprises a targeting moiety conjugated to the Oligo-HES complex through a linker and the conjugate comprises a therapeutic oligonucleotide selected from siRNA, shRNA, miRNA, an antagmir, a dicer substrate, an antisense oligonucleotide, and a expression cassette (e.g., plasmid) capable of expressing an siRNA, a miRNA, a ribozyme or an antisense oligonucleotide.

In some embodiments, the disclosure provides a T-Oligo-HES conjugate having the structure of formula (I)

$$\text{T-} {\left({\text{L}}_{\text{n}} \text{-} {\left(\text{Oligo-HES}\right)}_{\text{x}}\right)}_{\text{p}}$$

wherein:
   T is a targeting moiety that selectively binds a target of interest;
   L is a linker;
   Oligo-HES is an oligonucleotide complex containing a therapeutic oligonucleotide and an H-type excitonic structure (HES);
   n is 0 or 1;
   x is 1 to 30, 1-20, 1-10, or 1-5; and
   p is 1 to 30, 1-20, 1-10, or 1-5.

In some embodiments, the T-Oligo-HES conjugate having the structure of formula (I) comprises a therapeutic oligonucleotide that specifically hybridizes to a nucleic acid sequence in vivo and modulates the level of a protein encoded or regulated by the nucleic acid.

In some embodiments, the T-Oligo-HES conjugate having the structure of formula (I) comprises a therapeutic oligonucleotide that contains 1, 2, or 3 substitutions, deletions, or insertions, compared to the corresponding reverse complementary strand of the nucleic acid sequence.

In some embodiments, the disclosure provides a T-Oligo-HES conjugate having the structure of formula (II)

$$\text{T-} {\left[{\text{L}}_{\text{n}} \text{-} {\left\{{\left({\left(\text{Oligo2-LL}\right)}_{\text{m}} \text{-Oligo1-HES}\right)}_{\text{s}}\right\}}_{\text{t}}\right]}_{\text{u}}$$

wherein:
   T is a targeting moiety that selectively binds a target of interest;
   L is a linker;
   LL is a linker, optionally wherein LL is an alkyl such as a C6, C10, or C18 alkyl;
   Oligo1-HES is an oligonucleotide complex containing oligonucleotide 1 (Oligol) and an H-type excitonic structure (HES);
   Oligo2 is an oligonucleotide that may be the same or different from Oligol;
   n is 0 or 1;
   m is 0 or 1;
   s is 1 or 2;
   t is 1 or 2; and
   u is 1, 2, 3, 4, or 5.

In some embodiments, the T-Oligo-HES conjugate having the structure of formula (II) comprises a therapeutic oligonucleotide that specifically hybridizes to a nucleic acid sequence in vivo and modulates the level of a protein encoded or regulated by the nucleic acid.

In some embodiments, the T-Oligo-HES conjugate having the structure of formula (II) comprises a therapeutic oligonucleotide that contains 1, 2, or 3 substitutions, deletions, or insertions, compared to the corresponding reverse complementary strand of the nucleic acid sequence.

Where aspects or embodiments, disclosed herein are described in terms of a Markush group or other grouping of alternatives, the disclosure encompasses not only the entire group listed as a whole, but also each member of the group individually and all possible subgroups of the main group, and also the main group absent one or more of the group members. The disclosure also envisages the explicit exclusion of one or more of any of the group members.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### H-type Excitonic Structures (HES)

A “chromophore” is a group, substructure, or molecule that is responsible for the absorbance of light. Typical chromophores each have a characteristic absorbance spectrum.

A “fluorophore” is a chromophore that absorbs light at a characteristic wavelength and then re-emits the light most typically at a characteristic different wavelength. Fluorophores are well known to those of skill in the art and include, but are not limited to xanthenes and xanthene derivatives (xanthenes include fluoresceins and derivatives thereof), cyanines and cyanine derivatives (e.g., indocarbocyanine, an indodicarbocyanine), coumarins and coumarin derivatives, and chelators with the lanthanide ion series. A fluorophore is distinguished from a chromophore which absorbs, but does not characteristically re-emit light.

An “H-type excitonic structure” (HES) refers to two or more fluorophores whose transition dipoles are arranged in a parallel configuration resulting in a splitting of the excited singlet state; transitions between a ground state and an upper excited state are considered allowed and transitions between a ground state and lower excited state forbidden. HES formation in connection with certain fluorophores is known in the art and the disclosure encompasses the attachment of these fluorophores to oligonucleotides (e.g., diagnostic and therapeutic oligonucleotides) and the use of the resulting Oligo-HES complexes in T-Oligo-HES conjugates according to the methods described herein. Examples of HES forming fluorophores that can be contained in the T-Oligo-HES conjugates and used in the methods provided herein include, but are not limited to, xanthenes and xanthene derivatives, cyanine and cyanine derivatives, coumarins and chelators with the lanthanide ion series. In some embodiments, at least one fluorophore in the HES has an excitation and/or emission maxima from 350 to 800 nm. In some embodiments, at least two fluorophore in the HES have an excitation and/or emission maxima from 350 to 800 nm.

The terms “Oliognucleotide-HES” complex, “Oligo-HES” complex, and “HES-oligonucleotide” complex are used interchangeably herein to refer to a complex of one or more oligonucleotide strands (e.g., a single strand, double strand, triple strand or a further plurality of strands of linear or circular oligonucleotides containing the same, complementary or distinct oligonucleotide sequences) that contain 2 or more fluorphores that form an HES. The fluorophores of the HES-oligonucleotide may be attached at the 5′ and/or 3′ terminal backbone phosphates and/or at another base within an oligonucleotide or in different oligonucleotides so long as the collective HES-oligonucleotide contains one or more HES. The fluorophores are optionally attached to the oligonucleotide via a linker, such as a flexible aliphatic chain. Oliognucleotide-HES complexes and their use uses are further described in Intl. Appl. Publ. No. WO2014201306A1, the contents of which is herein incorporated by reference in its entirety and for all purposes.

An Oligo-HES may contain 1, 2, 3, 4, or more HES. Additionally, a HES in an HES-oligonucleotide may contain 2, 3, 4 or more of the same or different fluorophores. See, e.g., Toptygin et al., Chem. Phys. Lett. 277:430-435 (1997). In some embodiments, an HES is formed as a consequence of fluorophore aggregates between HES-oligonucleotides. In some embodiments, an HES is formed as a consequence of fluorophore aggregates between oligonucleotides that are singly labeled with a fluorophore capable of forming a HES.

The fluorophores in the Oligo-HES complexes contained in the provided T-Oligo-HES conjugates can be any fluorophores in the complex that are capable of forming an HES with a homotypic or heterotypic cognate fluorophore(s) in the complex. In some embodiments, the Oligo-HES complex comprises 2 fluorophores capable of forming an H-type excitonic structure. In some embodiments, the Oligo-HES complex comprises at least 1 fluorophore with an excitation and/or emission from 300-850 nm. In additional embodiments, the Oligo-HES complex comprises 2, 3, 4 or more fluorophores capable of forming an H-type excitonic structure. In further embodiments, Oligo-HES complex in the T-Oligo-HES conjugates comprise 2, 3, 4 or more fluorophore with an excitation and/or emission from 300-850 nm. In further embodiments, the Oligo-HES complex contains from about 2-20, from about 2-10, from about 2-6, or from about 2-4 fluorophores capable of forming an H-type excitonic structure. In additional embodiments, the Oligo-HES complex comprises 2, 3, 4, 5 or more fluorophores capable of forming one or more H-type excitonic structure. In further embodiments, the Oligo-HES complex comprises 2, 3, 4, 5 or more fluorophores with an excitation and/or emission from 300-850 nm. Two or more fluorophores are said to quench each other in an HES when their aggregate fluorescence is detectably less than the aggregate fluorescence of the fluorophores when they are separated, e.g., in solution at approximately 1 uM or less. The maximum of an HES absorbance spectrum as compared with spectra of the individual fluorophores shows the maximum absorbance wavelength to be shifted to a shorter wavelength, i.e., a blue shift. Fluorescence intensity of H-type Excitonic Structures or aggregates (herein “HES”) exhibits an intensity less than those of its components. Either a blue shift in the absorbance spectrum or a decrease in fluorescence intensity behavior of the H-type excitonic structures or aggregates can be utilized as an indicator of a signal reporter moiety. In preferred embodiments, two or more fluorophores in the Oligo-HES complex of the T-Oligo-HES conjugate increase or quench by at least 50%, preferably by at least 70%, more preferably by at least 80%, and most preferably by at least 90%, 95%, or even at least 99%. Examples of fluorophores that can form H-type excitonic structures include but are not limited to xanthenes, indocarbocyanines, indodicarbocyanines, and coumarins. In particular embodiments, the HES of an Oligo-HES complex contained in a T-Oligo-HES conjugate provided herein, contains at least one fluorophore that is a xanthene, indocarbocyanine, indodicarbocyanine, or a coumarin.

In some embodiments, the Oligo-HES complex of the T-Oligo-HES conjugate contains a fluorophore selected from: carboxyrhodamine 110, carboxytetramethylrhodamine, carboxyrhodamine-X, diethylaminocoumarin and an N-ethyl-N’-[5-(N″-succinimidyloxycarbonyl)pentyl]indocarbocyanine chloride, N-ethyl-N′-[5-(N″-succinimidyloxycarbonyl)pentyl]-3,3,3′,3′-tetramethyl-2′,2′-indodicarbocyanine chloride dye, and a Cy7 NHS Ester.

In further embodiments, the Oligo-HES complex contained in the T-Oligo-HES conjugate contains a fluorophore selected from: Rhodamine Green™ carboxylic acid, succinimidyl ester or hydrochloride; Rhodamine Green™ carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl) sulfonerhodamine or di(succinimidyl, ester); 5-(and-6)-carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethyl ammonium) salt; 5-carboxytetramethylrhodamine; 6-carboxytetramethylrhodamine; 5-(and-6)-carboxytetramethylrhodamine; 5-carboxytetra methylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; Rhodamine Red™-X succinimidyl ester; 6-(tetramethylrhodamine-5-(and-6)-carboxamido)hexanoic acid succinimidyl ester; tetramethylrhodamine-5-isothiocyanate; tetramethylrhodamine-6-isothiocyanate; tetramethylrhodamine-5- (and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; X-rhodamine-5-(and-6)-isothiocyanate; and the carbocyanines.

In some embodiments, Oligo-HES complex contained in the T-Oligo-HES conjugate contains a hetero-HES composed of different fluorophores. In particular embodiments, the hetero-HES contains a rhodamine or rhodamine derivative and a fluorescein or a fluorescein derivative or two carbocyanines. In further embodiments, the hetero-HES contains a fluorescein or fluorescein derivative selected from: 6-carboxy-4′,5′-dichloro-2′,7\′-dimethoxyfluorescein succinimidyl ester; 5-(and-6)-carboxyeosin; 5-carboxyfluorescein; 6-carboxyfluorescein; 5-(and-6)-carboxyfluorescein; 5-carboxyfluorescein-bis-(5-carboxymethoxy-2-nitrobenzyl)ether, -alanine-carboxamide, or succinimidyl ester; 5-carboxyfluorescein succinimidyl ester; 6-carboxyfluorescein succinimidyl ester, 5-(and-6)-carboxyfluorescein succinimidyl ester; 5-(4,6-dichlorotriazinyl) aminofluorescein; 2′\,7′-difluorofluorescein; eosin-5-isothiocyanate; erythrosin-5-isothiocyanate; 6-(fluorescein-5-carboxamido) hexanoic acid or succinimidyl ester; 6-(fluorescein-5-(and-6)-carboxamido) hexanoic acid or succinimidyl ester; fluorescein-5-EX succinimidyl ester; fluorescein-5-isothiocyanate; and fluorescein-6-isothiocyanate.

In some embodiments, at least one Oligo-HES complex contained in the T-Oligo-HES conjugate contains a fluorophores with excitation and/or emission maxima from 350 to 800 nm. In some embodiments, at least two Oligo-HES complexes contained in the T-Oligo-HES conjugate contain a fluorophore with excitation and/or emission maxima from 350 to 800 nm. In some embodiments, all the Oligo-HES complexes contained in the T-Oligo-HES conjugate contain a fluorophore with excitation and/or emission maxima from 350 to 800 nm. In further embodiments, all of the fluorophores in the T-Oligo-HES conjugate have an excitation and/or emission maxima from 350 to 800 nm.

H-type Excitonic Structures HES and fluorophores capable of forming HES are further described in Intl. Appl. Publ. No. WO2014201306A1, the contents of which are herein incorporated by reference in its entirety and for all purposes.

### Oligonucleotides

The term “oligonucleotide” or “Oligo” as used herein refers to an oligomer or polymer of ribonucleic acid (RNA), deoxyribonucleic acid (DNA) or a mimetic thereof. This term includes oligonucleotides composed of naturally-occurring nucleobases, sugars and covalent internucleoside (backbone) linkages (i.e., “unmodified oligonucleotide), as well as oligomeric compounds having non-naturally-occurring nucleobases, sugars and/or internucleoside linkages and/or analogs of DNA and/or RNA which function in a similar manner (i.e., nucleic acid “mimetics” or “mimics”). Such mimetic oligonucleotides are often preferred over native forms because of desirable properties such as: enhanced affinity for nucleic acid target and increased stability in the presence of nucleases. For example, as used herein, the term “oligonucleotide” includes morpholino (MNO) wherein one or more ribose rings of the nucleotide backbone is replaced with a morpholine ring and phosphorodiamidate morpholino oligomers (PMOs) wherein one or more ribose ring of the nucleotide backbone is replaced with a morpholine ring and the negatively charged intersubunit linkages are replaced by uncharged phosphorodiamidate linkages. Likewise, the term oligonucleotide encompasses PNAs in which one or more sugar phosphate backbone of an oligonucleotide is replaced with an amide containing backbone. For the purposes of this specification, and as sometimes referenced in the art, modified oligonucleotides that do not have a phosphorus atom in their internucleoside backbone can also be considered to be oligonucleosides. Moreover, the oligonucleotides may be referred to as oligomers

The delivery of oligonucleotides using the T-Oligo-HES conjugates provided herein is sequence independent and accordingly, the oligonucleotides contained in the T-Oligo-HES conjugate vehicles can be any form of nucleic acid or mimetic that is known to be desirable to introduce into a cell.

Oligonucleotides in the Oligo-HES complex of the T-Oligo-HES conjugate can be in the form of single-stranded, double-stranded, circular or hairpin oligonucleotides. In some embodiments, the oligonucleotides are single-stranded DNA, RNA, or a nucleic acid mimetic (e.g., PMO, MNO, PNA, or oligonucleotides containing one or more modified nucleotides such as a 2′OME and LNA). In some embodiments, the oligonucleotides are double-stranded DNA, RNA, nucleic acid mimetic, DNA/nucleic acid mimetic, DNA-RNA and RNA-nucleic acid mimetic.

The inventors have surprisingly discovered that complexes containing Oligo-HES such as ssDNA and dsRNA display superior sequence independent intracellular delivery that require the administration of orders of magnitude of less oligonucleotides than that required by conventional oligonucleotide delivery vehicles. Examples of single-stranded nucleic acids contained in the complexes and T-Oligo-HES conjugates provided herein include, but are not limited to, antisense, siRNA, shRNA, ribozymes, miRNA, anti-miRNA, triplex-forming oligonucleotides and aptamers.

In some embodiments, an oligonucleotide in an Oligo-HES complex of the T-Oligo-HES conjugate is single stranded DNA (ssDNA). In preferred embodiments, at least a portion of the ssDNA oligonucleotide specifically hybridizes with a target RNA to form an oligonucleotide-RNA duplex. In further preferred embodiments, the oligonucleotide-RNA duplex is susceptible to an RNase cleavage mechanism (e.g., RNase H). In some embodiments, a single stranded oligonucleotide in the complex comprises at least one modified backbone linkage, at least one modified sugar, and/or at least one modified nucleobase (e.g., as described herein). In some embodiments, a single stranded oligonucleotide in the complex comprises at least one modified backbone linkage, at least one modified sugar, and/or at least one modified nucleobase (e.g., as described herein) and is capable of forming an oligonucleotide-RNA duplex that is susceptible to an RNase cleavage mechanism. In particular embodiments, the single stranded oligonucleotide is a gapmer (i.e., as described herein or otherwise known in the art). In additional embodiments, the T-Oligo-HES conjugate contains an oligonucleotide comprising at least one modified backbone linkage, at least one modified sugar, and/or at least one modified nucleobase that decreases the sensitivity of the oligonucleotide to an RNase cleavage mechanism (e.g., as described herein). In particular embodiments, the single stranded oligonucleotide comprises at least one 2′OME, LNA, MNO or PNA motif

Examples of double-stranded DNA oligonucleotides contained in an Oligo-HES complex of T-Oligo-HES conjugates provided herein include, but are not limited to, dsRNAi and dicer substrates and other RNA interference reagents, and sequences corresponding to structural genes and/or control and termination regions.

In some embodiments, an oligonucleotide contained in the Oligo-HES complex of a T-Oligo-HES conjugate is a linear double-stranded RNA (dsRNA). In preferred embodiments, the ds-RNA is susceptible to an RNase cleavage mechanism (e.g., Dicer and Drosha (an RNase III enzyme)). In additional embodiments, the dsRNA is able to be inserted into the RNA Induced Silencing Complex (RISC) of a cell. In further embodiments, a RNA strand of the dsRNA is able to use the RISC complex to effect cleavage of an RNA target.

In additional embodiments, an oligonucleotide contained in the Oligo-HES complex of a T-Oligo-HES conjugate is a double stranded oligonucleotide in which one or both oligonucleotides contain at least one modified backbone linkage, at least one modified sugar, and/or at least one modified nucleobase. In preferred embodiments, the double strand oligonucleotide is susceptible to an RNase cleavage mechanism (e.g., Dicer and Drosha (an RNase III enzyme). In additional embodiments, the double stranded oligonucleotide is able to be inserted into the RNA Induced Silencing Complex (RISC) of a cell. In further embodiments, an oligonucleotide strand of the double stranded oligonucleotide is able to use the RISC complex to effect cleavage of an RNA target.

In further embodiments, an oligonucleotide contained in the Oligo-HES complex of a T- Oligo-HES conjugate is a triple-stranded DNA/RNA chimeric. In some embodiments, the oligonucleotide complex contains at least one oligonucleotide comprising at least one modified backbone linkage, at least one modified sugar, and/or at least one modified nucleobase. In particular embodiments, at least one oligonucleotide in the complex comprises at least one 2′OME, LNA, MNO or PNA motif.

Oligonucleotides in the T-Oligo-HES conjugates provided herein are routinely prepared linearly but can be joined or otherwise prepared to be circular and may also include branching. Separate oligonucleotides can specifically hybridize to form double stranded compounds that can be blunt-ended or may include overhangs on one or both termini. In particular embodiments, double stranded oligonucleotides (e.g., dsRNA and double stranded oligonucleotide in which at least one of the oligonucleotide strands is a nucleic acid mimetic) contained in the contained in the Oligo-HES complex of a T- Oligo-HES conjugate provided herein are between 21-25 nucleotides in length and have 1, 2, or 3 nucleotide overhangs at either or both ends.

Oligonucleotides contained in the Oligo-HES complex of a T- Oligo-HES conjugate provided herein may be of various lengths, generally dependent upon the particular form of nucleic acid or mimetic and its intended use. In some embodiments, nucleic acid/ oligonucleotides in the Oligo-HES complex range from about 5 nucleotides to about 500 nucleotides, and preferably from about 10 nucleotides to about 100 nucleotides in length.

In some embodiments, the Oligo-HES complex of a T- Oligo-HES conjugate contains an oligonucleotide comprising at least 8 contiguous nucleobases that are complementary to a target nucleic acid sequence. In various related embodiments, an oligonucleotide in the Oligo-HES complex is from about 8 to about 100 monomeric subunits (used interchangeably with the term “nucleotides” herein) or from about 8 to about 50 nucleotides in length.

In additional embodiments, an oligonucleotide in the Oligo-HES complex of a T- Oligo-HES conjugate ranges in length from about 8 to about 30 nucleotides, from about 15 to about 30 nucleotides, from about 20 to about 30 nucleotides, from about 18 to 26 nucleotides, from about 19 to 25 nucleotides, from about 20 to 25 or from about 21 to 25 nucleotides.

In further embodiments, an oligonucleotide in the Oligo-HES complex of a T- Oligo-HES conjugate is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 subunits (nucleotides) in length. In particular embodiments, the oligonucleotides are 19, 20, 21, 22, 23, 24 or 25 nucleotides in length.

In particular embodiments, an oligonucleotide in the Oligo-HES complex of a T- Oligo-HES conjugate contains a double strand of RNA oligonucleotides of between 21-25 nucleotides in length and have 1, 2, or 3 nucleotide overhangs at either or both ends. In other embodiments, the oligonucleotide in complex contains a double strand of oligonucleotides in which at least one of the oligonucleotide strands is a nucleic acid mimetic of between 21-25 nucleotides in length and the double stranded oligonucleotide has a 1, 2, or 3 nucleotide overhang at either or both ends.

### Oligonucleotides Containing Modifications

Oligonucleotide in the Oligo-HES complex of a T- Oligo-HES conjugate provided herein preferably include one or more modified internucleoside linkages, modified sugar moieties and/or modified nucleobases. Such modified oligonucleotides (i.e., mimetics) are typically preferred over native forms because of desirable properties including for example, enhanced cellular uptake, enhanced affinity for nucleic acid target, increased stability in the presence of nucleases and/or increased inhibitory activity.

### Modified Internucleoside Linkages

The term “oligonucleotide” as used herein, refers to those oligonucleotides that retain a phosphorus atom in their internucleoside backbone as well as those that do not have a phosphorus atom in their internucleoside backbone.

In some embodiments, an oligonucleotide in the Oligo-HES complex of a T- Oligo-HES conjugate comprise one or more modified internucleoside linkages. Modified internucleoside linkages in the oligonucleotides of the complexes and conjugates provided herein may include for example, any manner of internucleoside linkages known to provide enhanced nuclease stability to oligonucleotides relative to that provided by phosphodiester internucleoside linkages. Oligonucleotides having modified internucleoside linkages include internucleoside linkages that retain a phosphorus atom as well as internucleoside linkages that do not contain phosphorus. In some embodiments, the oligonucleotides comprise modified internucleoside linkages that alternate between modified and unmodified internucleoside linkages. In some embodiments, most of the internucleoside linkages in the oligonucleotide are modified. In further embodiments, every internucleoside linkage in the oligonucleotide is modified.

Preferred modified oligonucleotide backbones include, for example, phosphorothioates, chiral phosphorothioates, phosphorodithioates, phosphodiesters, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e., a single inverted nucleoside residue which may be abasic (the nucleobase is missing or has a hydroxyl group in place thereof). Various salts, mixed salts and free acid forms are also included.

In preferred embodiments, an oligonucleotide in the Oligo-HES complex of a T- Oligo-HES conjugate includes at least one phosphorothioate (PS) internucleoside linkage wherein one of the nonbridging oxygen atoms in the phosphodiester bond is replaced by sulfur. Oligonucleotides containing PS internucleoside linkage form regular Watson-Crick base pairs, activate RNase H, carry negative charges for cell delivery and display other additional desirable pharmacokinetic properties. In some embodiments, the at least one modified internucleoside linkage is phosphorothioate. In some embodiments, at least 2, 3, 4, 5, 10 or 15 of the internucleoside linkages contained in the oligonucleotide is a phosphorothioate linkage. In some embodiments, at least 1-10, 1-20, 1-30 of the modified internucleoside linkages is a phosphorothioate linkage. In some embodiments, at least 2, 3, 4, 5, 10 or 15 of the modified internucleoside linkages is a phosphorothioate linkage. In additional embodiments, each internucleoside linkage of an oligonucleotide is a phosphorothioate internucleoside linkage.

In some embodiments, an oligonucleotide in the Oligo-HES complex of a T- Oligo-HES conjugate contains a 8 to 14 base PS-modified deoxynucleotide ‘gap’ flanked on either end with 2 to 5 MOE nucleotides (i.e., a MOE gapmer). In some embodiments, the T-Oligo-HES conjugates provided herein have an oligonucleotide containing a 8 to 14 base PS-modified deoxynucleotide ‘gap’ flanked on either end with 2 to 5 LNA nucleotides (i.e., a LNA gapmer). In additional embodiments, the Oligo-HES complex has an oligonucleotide containing a 8 to 14 base PS-modified deoxynucleotide ‘gap’ flanked on either end with 2 to 5 tricyclo-DNA nucleotides (i.e., a tcDNA gapmer).

Another suitable phosphorus-containing modified internucleoside linkage is the N3′-P5′ phosphoroamidates (NPs) in which the 3′-hydroxyl group of the 2′-deoxyribose ring is replaced by a 3′-amino group. Oligonucleotides containing NPs internucleoside linkages exhibit high affinity towards complementary RNA and resistance to nucleases. Since phosphoroamidate do not induce RNase H cleavage of the target RNA, oligonucleotides containing these internucleoside linkages have applications in those instances where RNA integrity needs to be maintained, such as those instances in which the oligonucleotides modulation mRNA splicing. In some embodiments, at least 2, 3, 4, 5, 10 or 15 of the internucleoside linkages in an oligonucleotide contained in a T- Oligo-HES conjugate is a phosphoroamidate linkage. In some embodiments, at least 1-10, 1-20, 1-30 of the modified internucleoside linkages is a phosphoroamidate linkage. In some embodiments, at least 2, 3, 4, 5, 10 or 15 of the modified internucleoside linkages is a phosphoroamidates linkage. In additional embodiments, each internucleoside linkage of an antisense compound is a phosphoroamidate internucleoside linkage.

Numerous modified internucleoside linkages and their method of synthesis are known in the art and encompassed by the modifications that may be contained in the oligonucleotides of the T-Oligo-HES conjugates. Exemplary U.S. patents that teach the preparation of phosphorus-containing internucleoside linkages include, but are not limited to, U.S. Pat. Nos. 3,687,808; 4,469,863; 4,476,301; 5,023,243; 5,177,196; 5,188,897; 5,194,599; 5,264,423; 5,276,019; 5,278,302; 5,286,717; 5,321,131; 5,399,676; 5,405,939; 5,489,677; 5,453,496; 5,455,233; 5,466,677; 5,476,925; 5,519,126; 5,527,899; 5,536,821; 5,541,306; 5,550,111; 5,563,253; 5,565,555; 5,602,240; 5,571,799; 5,587,361; 5,625,050; 5,646,269; 5,663,312; 5,672,697; 5,677,439; and 5,721,218; each of which is herein incorporated by reference in its entirety.

T-Oligo-HES conjugates containing oligonucleotides that do not include a phosphorus atom are also provided herein. Examples of such oligonucleotides include those containing backbones formed by short chain alkyl or cycloalkyl internucleoside linkages, mixed heteroatom and alkyl or cycloalkyl internucleoside linkages, or one or more short chain heteroatomic or heterocyclic internucleoside linkages. These modified backbones include, but are not limited to oligonucleotides having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts. Methods of making oligonucleotides containing backbones that do not include a phosphorous atom are known in the art and include, but are not limited to, those methods and compositions disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,646,269; 5,663,312; 5,633,360; 5,677,437; 5,677,439; 5,792,608; and each of which is herein incorporated by reference in its entirety.

In some embodiments, the oligonucleotides in the T-Oligo-HES conjugate contains one or more modified backbone linkages selected from: 3′-methylene phosphonate, methylene (methylimino) (also known as MMI), morpholino, locked nucleic acid, and a peptide nucleic acid linkage. The modified backbone linkages may be uniform or may be alternated with other linkages, particularly phosphodiester or phosphorothioate linkages, as long as RNAse H cleavage is not supported.

In some embodiments, the oligonucleotides in the T-Oligo-HES conjugate contains oligonucleotides that are nucleic acid mimetics. The term mimetic as it is applied to oligonucleotides is intended to include oligonucleotides wherein the sugar or both the sugar and the internucleotide linkage are replaced with alternative groups.

In some embodiments, the T-Oligo-HES conjugate contains an oligonucleotide having one or more morpholino linkages. The RNAse and nuclease resistant properties of morpholinos make them particularly useful in regulating transcription in a cell. Accordingly, in some embodiments, a complex containing a morpholino unit is used to modulate gene expression. In some embodiments, morpholino unit is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino. In particular embodiments, each monomeric unit of the oligonucleotide corresponds to a phosphorodiamidate morpholino (PMO). In additional embodiments, a T-Oligo-HES conjugate containing a morpholino oligonucleotide (e.g., PMO) is used to alter mRNA splicing in a subject. In additional embodiments, a T-Oligo-HES conjugate containing an oligonucleotide comprising one or more morpholino nucleobases such as a PMO, is used as an antisense an agent.

In additional embodiments, the T-Oligo-HES conjugate contains an oligonucleotide that is a peptide nucleic acid (PNA). PNAs are nucleic acid mimetics in which the sugar phosphate backbone of an oligonucleotide is replaced with an amide containing backbone. In particular embodiments, the phosphate backbone of the oligonucleotide is replaced with an aminoethylglycine backbone and the nucleobases are bound directly or indirectly to aza nitrogen atoms of the amide portion of the backbone. Numerous PNAs and methods of making PNAs are known in the art (see, e.g., Nielsen et al., Science, 254: 1497-150 (1991), and U.S. Pat. Nos. 5,539,082; 5,714,331; and 5,719,262, each of which is herein incorporated by reference in its entirety. PNA containing oligonucleotides provide increased stability and favorable hybridization kinetics and have a higher affinity for RNA than DNA compared to unsubstituted counterpart nucleic acids and do not activate RNAse H mediated degradation. Oligonucleotides that can be contained in the T-Oligo-HES conjugate includes PNA analogues including PNAs having modified backbones with positively charged groups and/or one or more chiral constrained stereogenic centers at the C2(alpha), such as a D-amino acid, or C5(gamma), such as an L-amino acid (e.g., L-lysine) position of one or more monomeric units of the oligonucleotide.

The RNAse and nuclease resistant properties of PNA oligonucleotides make them particularly useful in regulating RNA (e.g., mRNA and miRNA) in a cell via a steric block mechanism. In some embodiments, an oligonucleotide in the T-Oligo-HES conjugate comprises at least one PNA oligonucleotide. In some embodiments, the oligonucleotide contains an oligonucleotide comprising at least one PNA oligonucleotide and modulates gene expression by strand invasion of chromosomal duplex DNA. In a further embodiment, the T-Oligo-HES conjugate contains an oligonucleotide comprising at least one PNA oligonucleotide and alters mRNA splicing in a subject. In additional embodiments, the T-Oligo-HES conjugate contains an oligonucleotide comprising at least one PNA oligonucleotide such as, a PMO, and acts as an antisense.

Similarly, the RNAse and nuclease resistant properties of morpholino containing oligonucleotides make these oligonucleotides useful in regulating RNA (e.g., mRNA and miRNA) in a cell via a steric block mechanism. In some embodiments, the T-Oligo-HES conjugate contains an oligonucleotide comprising at least one morpholino oligonucleotide such as, a PMO, and modulate gene expression by strand invasion of chromosomal duplex DNA. In a further embodiment, the oligonucleotide comprises at least one morpholino such as, a PMO, and alters mRNA splicing in a subject. In additional embodiments, the oligonucleotide comprises at least one morpholino oligonucleotide such as, a PMO, and act as an antisense.

Additionally, the RNAse and nuclease resistant properties of bicyclic sugar-containing nucleotides make these oligonucleotides useful in regulating RNA (e.g., mRNA and miRNA) in a cell via a steric block mechanism. In some embodiments, the T-Oligo-HES conjugate contains an oligonucleotide comprising at least one bicyclic sugar containing nucleotide. In some embodiments, the bicyclic sugar containing nucleotide is a locked nucleic acid (LNA). In further embodiments, the LNA has a 2′-hydroxyl group linked to the 3′ or 4′ carbon atom of the sugar ring. In a further embodiment, the oligonucleotide comprises at least one locked nucleic acid (LNA) in which a methylene (--CH2--)n group bridges the 2′ oxygen atom and the 4′ carbon atom wherein n is 1 or 2. In some embodiments, the T-Oligo-HES conjugate contains an oligonucleotide comprising at least one bicyclic sugar containing nucleotide such as an LNA, and modulates gene expression by strand invasion of chromosomal duplex DNA. In other embodiments, the oligonucleotide comprises at least one bicyclic sugar, such as an LNA, and alters mRNA splicing in a subject. In additional embodiments, the oligonucleotide comprises at least one bicyclic sugar oligonucleotide, such as an LNA, and act as an antisense.

### Modified Sugar Moieties

In some embodiments, an oligonucleotides in the T-Oligo-HES conjugate comprises one or more nucleosides having one or more modified sugar moieties which are structurally distinguishable from, yet functionally interchangeable with, naturally occurring or synthetic unmodified nucleobases. In further embodiments, the T-Oligo-HES conjugate contains an oligonucleotide comprising a modified sugar at each nucleoside (unit).

Examples of sugar modifications useful in the oligonucleotides contained in T-Oligo-HES conjugates include, but are not limited to, compounds comprising a sugar substituent group selected from: OH; F; O—, S—, or N-alkyl; or O-alkyl-O-alkyl, wherein the alkyl, alkenyl and alkynyl may be substituted or unsubstituted C1 to C10 alkyl or C2 to C10 alkenyl and alkynyl.

Representative modified sugars include carbocyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. Examples of 2′-sugar substituent groups useful in the oligonucleotides include, but are not limited to: OH; F; O—, S—, or N-alkyl; O—, S—, or N-alkenyl; allyl, amino; azido; thio; O-allyl; O(CH2)2SCH3; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. In particular embodiments, the oligonucleotides contain at least one 2′-sugar substituent group selected from: O[(CH2)nO]mCH3, O(CH2)nOCH3, O(CH2)nNH2, O(CH2)nCH3, O(CH2)nONH2, and O(CH2)nON[(CH2)nCH3)]2, where n and m are from 1 to about 10. Other preferred oligonucleotides contain at least one 2′-sugar substituent group selected from: a C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligonucleotide compound, and other substituents having similar properties.

In particular embodiments, an oligonucleotide in the T-Oligo-HES conjugate comprises at least one 2′-substituted sugar having a 2′-methoxyethoxy (2′-O—CH2CH2OCH3, aka 2′-MOE) substituent group.

In some embodiments, an oligonucleotide in the T-Oligo-HES conjugate comprises at least one 2′-modified nucleoside selected from: 2′-allyl (2′-CH2—CH—CH2), 2′-O-allyl (2′-O—CH2—CH—CH2), 2′-aminopropoxy (2′-OCH2CH2CH2NH2), and 2′-acetamido (2′-O—CH2C(—O)NR1R1 wherein each R1 is independently, H or Cl-Cl alkyl.

In further embodiments, an oligonucleotide in the T-Oligo-HES conjugate comprises at least one 2′-substituted sugar having: a 2′-dimethylaminooxyethoxy (2′-O(CH2)2ON(CH3)2 group, also known as 2′-DMAOE) substituent group; a 2′-dimethylaminoethoxyethoxy (2′-O—CH2—O—CH2—N(CH2)2, also known as 2′-O-dimethylaminoethoxyethyl or 2′-DMAEOE) substituent group; or a 2′-O-methyl (2′-O--CH3) substituent group. In further embodiments, an oligonucleotide in the T-Oligo-HES conjugate comprises least one 2′-substituted sugar having a 2′-fluoro (2′-F) substituent group.

In some embodiments, an oligonucleotide in the T-Oligo-HES conjugate comprises at least one bicyclic sugar. In specific embodiments, the oligonucleotide has at least one locked nucleic acid (LNA) in which the 2′-hydroxyl group is linked to the 3′ or 4′ carbon atom of the sugar ring. In a particular embodiment, the oligonucleotide comprises at least one locked nucleic acid (LNA) in which a methylene (—CH2—)n group bridges the 2′ oxygen atom and the 4′ carbon atom wherein n is 1 or 2. In another embodiment, the oligonucleotide contains at least one bicyclic modified nucleoside having a bridge between the 4′ and the 2′ ribosyl ring atoms wherein the bridge is selected: 4′-(CH2)—O-2′ (LNA); 4′-(CH2)—S-2; 4′-(CH2)2—O-2′ (ENA); 4′-C(CH3)2—O-2′; 4′-CH(CH3)—O-2′; 4′-CH(CH2OCH3)—O-2′; 4′-CH2—N(OCH3)-2′; 4′-CH2—O—N(CH3)-2′; 4′-CH2—N(R)—O-2′; 4′-CH2—CH(CH3)-2′ and 4′-CH2—C(—CH2)-2′, wherein R is independently, H, a C1-C12 alkyl, or a protecting group. In some embodiments, an oligonucleotide in T-Oligo-HES conjugate comprises at least one of the foregoing sugar configurations and an additional motif such as, alpha-L-ribofuranose, beta-D-ribofuranose or alpha-L-methyleneoxy (4′-CH2—O-2′). Further LNAs useful in of the oligonucleotides provided herein and their preparation are known in the art. See, e.g., U.S. Pat. Nos. 6,268,490, 6,670,461, 7,217,805, 7,314,923, and 7,399,845; WO 98/39352 and WO 99/14226; and Singh et al., Chem. Commun. 4:455-456 (1998), the contents of each of which is herein incorporated by reference in its entirety.

In some embodiments, an oligonucleotide in the T-Oligo-HES conjugate comprises a chemically modified furanosyl (e.g., ribofuranose) ring moiety. Examples of chemically modified ribofuranose rings include, but are not limited to, addition of substituent groups (including 5′ and 2′ substituent groups, and particularly the 2′ position, bridging of non-geminal ring atoms to form bicyclic nucleic acids (BNA), replacement of the ribosyl ring oxygen atom with S, N(R), or C(R1)(R)2 (R--H, C1-C12 alkyl or a protecting group) and combinations thereof. Examples of chemically modified sugars include 2′-F-5′-methyl substituted nucleoside (see e.g., WO 2008/101157, for other +disclosed 5′,2′-bis substituted nucleosides) or replacement of the ribosyl ring oxygen atom with S with further substitution at the 2′-position (see e.g., US20050130923) or alternatively 5′-substitution of a BNA (WO 2007/134181 wherein LNA is substituted with for example, a 5′-methyl or a 5′-vinyl group).

T-Oligo-HES conjugates containing an oligonucleotide comprising at least one nucleotide having a similar modification to those described above, at the 3′ position of the sugar on the 3′ terminal nucleotide or in 2′-5′ linked oligonucleotides and the 5′ position of 5′ terminal nucleotide are also provided herein. Representative U.S. patents that teach the preparation of 2′-modified nucleosides contained in the provided oligonucleotides include, but are not limited to, U.S. Pat. Nos. 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; and 5,792,747, each of which is herein incorporated by reference in its entirety.

In some embodiments, the T-Oligo-HES conjugate contains an oligonucleotide comprising at least one heterocyclic bicyclic nucleic acid. For example, in some embodiments, the oligonucleotide has at least one ENA motif (see, e.g., WO 01/49687, the contents of which are herein incorporated by reference in its entirety).

In additional embodiments, the T-Oligo-HES conjugate contains an oligonucleotide comprising at least one replacement of a five-membered furanose ring by a six-membered ring. In at least one embodiment, the oligonucleotide has at least one cyclohexene nucleic acid (CeNAs).

In some embodiments, the T-Oligo-HES conjugate contains an oligonucleotide comprising at least one tricyclo-DNA (tcDNA). In additional embodiments, the oligonucleotide contains a 8 to 14 base PS-modified deoxynucleotide ‘gap’ flanked on either end with 2 to 5 tricyclo-DNA nucleotides (i.e., a tcDNA gapmer).

In particular embodiments, the T-Oligo-HES conjugate contains an oligonucleotide comprising a phosphorothioate backbone and/or an oligonucleosides with a heteroatom backbones, such as —CH2—NH—O—CH2—, —CH2—N(CH3)—O—CH2— (also known as a methylene (methylimino) or MMI backbone), —CH2—O—N(CH3)—CH2—, —CH2—N(CH3)—N(CH3)—CH2— and —O—N(CH3)—CH2—CH2—, and an amide backbone (see, e.g., U.S. Pat. No. 5,602,240). In additional embodiments, the T-Oligo-HES conjugate contains an oligonucleotide comprising a phosphorodiamidate backbone structure. In further embodiments, the T-Oligo-HES conjugate contains an oligonucleotide comprising a phosphorodiamidate morpholino (i.e., PMO) backbone structure (see, e.g., U.S. Pat. No. 5,034,506, the contents of which are incorporated herein in their entirety).

### Modified Nucleobases

The T-Oligo-HES conjugates may also contain an oligonucleotide comprising one or more nucleobase modifications which are structurally distinguishable from, yet functionally interchangeable with, naturally occurring or synthetic unmodified nucleobases.

The terms “unmodified” or “natural” nucleobases as used herein, include the purine bases adenine (A) and guanine (G), and the pyrimidine bases thymine (T), cytosine (C) and uracil (U). Modified nucleobases include synthetic and natural nucleobases such as, for example, 5-methylcytosine (5-me-C). In some embodiments, the T-Oligo-HES conjugate contains an oligonucleotide comprising at least one 5′ methylcytosine or a C-5 propyne. In some embodiments, each cytosine in the oligonucleotide is a methylcytosine.

Modified nucleobases are also referred to herein as heterocyclic base moieties and include other synthetic and natural nucleobases such as xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(--CC--CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 3-deazaguanine and 3-deazaadenine.

Heterocyclic base moieties contained in the oligonucleotides may also include those in which the purine or pyrimidine base is replaced with other heterocycles such as, 7-deazaadenine, 7-deazaguanosine, 2-aminopyridine and 2-pyridone. Nucleobases that are particularly useful for increasing the binding affinity of the provided oligonucleotides include 5-substituted pyrimidines, 6-azapyrimidines and N-2, N-6 and O-6 substituted purines, including 2 aminopropyladenine, 5-propynyluracil and 5-propynylcytosine.

Additional modified nucleobases that are optionally included in the oligonucleotide contained in a T-Oligo-HES conjugate, include, but are not limited to, tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-b][1,4]benzoxazin-2(3H)-one), carbazole cytidine (2H-pyrimido [4,5-b]indol-2-one), pyridoindole cytidine (H-pyrido [3′,2′:4,5] pyrrolo[2,3-d]pyrimidin-2-one), or guanidinium G-clamps and analogs. Representative guanidino substituent groups are disclosed in U.S. Pat. No. 6,593,466, which is hereby incorporated by reference in its entirety. Representative acetamido substituent groups are disclosed in U.S. Pat. No. 6,147,200, which is hereby incorporated by reference in its entirety.

Numerous modified nucleobases encompassed by the oligonucleotides contained in the T-Oligo-HES conjugates provided herein and their methods of synthesis are known in the art, and include, for example, the modified nucleobases disclosed in The Concise Encyclopedia Of Polymer Science And Engineering, pages 858-859, Kroschwitz, J. I., ed. John Wiley & Sons, 1990; Englisch et al., Angewandte Chemie, International Edition, 30:613 (1993); Sanghvi, Y. S., Chapter 15, Antisense Research and Applications, pages 289-302; Crooke, S. Ted., CRC Press, 1993; and U.S. Pat. Nos. 3,687,808; 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,681,941; 5,750,692; 5,830,653; 5,763,588; 6,005,096; 6,028,183 and 6,007,992 and U.S. Appl. Publ. No. 20030158403, each of which herein incorporated by reference in its entirety.

### Chimeric Oligonucleotides

In some embodiments, the T-Oligo-HES conjugates contain oligonucleotides comprising one or more modified internucleoside linkages, modified sugar moieties and/or modified nucleobases. In some embodiments, oligonucleotides are chimeric oligonucleotides (e.g., chimeric oligomeric compounds). The terms “chimeric oligonucleotides” or “chimeras” are oligonucleotides that contain at least 2 chemically distinct regions (i.e., patterns and/or orientations of motifs of chemically modified subunits arranged along the length of the oligonucleotide) each made up of at least one monomer unit, i.e., a nucleotide or nucleoside in the case of a nucleic acid based oligonucleotide compound. Chimeric oligonucleotides have also been referred to as for example, hybrids (e.g., fusions) and gapmers. Representative United States patents that teach the preparation of such chimeric oligonucleotide structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

Chimeric antisense compounds typically contain at least one region modified so as to confer increased resistance to nuclease degradation, increased cellular uptake, increased binding affinity for the target nucleic acid, and/or increased inhibitory activity. By way of example, gapmers are chimeric oligonucleotides comprising a contiguous sequence of nucleosides that is divided into 3 regions, a central region (gap) flanked by two external regions (wings). Gapmer design typically includes a central region of about 5-10 contiguous 2′-deoxynucleotides which serves as a substrate for RNase H is typically flanked by one or two regions of 2′-modified oligonucleotides that provide enhanced target RNA binding affinity, but do not support RNAse H cleavage of the target RNA molecule. Consequently, comparable results can often be obtained with shorter oligonucleotides having substrate regions when chimeras are used, compared to for example, phosphorothioate deoxyoligonucleotides hybridizing to the same target region. Other chimeric oligonucleotides rely on regions conferring for example, altered levels of binding affinity over the length of an oligonucleotide for its target including regions of modified nucleosides which exhibit either increased or decreased affinity as compared to the other regions. So called, “MOE-gapmers” have 2′-MOE modifications in the wings, often contain full PS backbones, and frequently include 5′MeC modifications on all cytosines.

Alternatively, for those situations in which RNAse H activity may be undesirable, such as in the modulation of RNA processing, it may be preferable to use uniformly modified oligonucleotides, such as designs using modified oligonucleotides that do not support RNAse H activity at each nucleotide or nucleoside position. As used herein the term “fully modified motif” is meant to include a contiguous sequence of sugar modified nucleosides wherein essentially each nucleoside is modified to have the same modified sugar moiety. Suitable sugar modified nucleosides for fully modified oligonucleotides include, but are not limited to, 2′-Fluoro (2′F), 2′-O(CH2)2OCH3 (2′-MOE), 2′-OCH3 (2′-O-methyl), and bicyclic sugar modified nucleosides. In one aspect the 3′ and 5′-terminal nucleosides are left unmodified. In a preferred embodiment, the modified nucleosides are either 2′-MOE, 2′-F, 2′-O—Me or a bicyclic sugar modified nucleoside.

In some embodiments, the provided T-Oligo-HES conjugates contain oligonucleotides that are modified to have one or more stabilizing groups. In some embodiments, the stabilizing groups are attached to one or both termini of the oligonucleotides to enhance properties such as, nuclease stability. In some embodiments, the stabilizing groups are cap structures. By “cap structure or terminal cap moiety” is meant chemical modifications, which have been incorporated at either terminus of oligonucleotides (see for example WO 97/26270, which is herein incorporated by reference in its entirety). These terminal modifications may serve to protect the oligonucleotides having terminal nucleic acid molecules from exonuclease degradation and/or may help in the delivery and/or localization of the oligonucleotide within a cell. The oligonucleotide may contain the cap at the 5′-terminus (5′-cap), the 3′-terminus (3′-cap), or both the 5′-terminus and the 3′-termini. In the case of double-stranded oligonucleotides, the cap may be present at either or both termini of either strand. Cap structures are known in the art and include, for example, inverted deoxy abasic caps. Further 3′ and 5′-stabilizing groups that can be used to cap one or both ends of an oligonucleotide (e.g., antisense) compound to impart nuclease stability include those disclosed in WO 03/004602, which is herein incorporated by reference in its entirety.

In some embodiments, the T-Oligo-HES conjugate comprises a 5′-cap and/or a structure that is an inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotide; alpha-nucleotide; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or a bridging or non-bridging methylphosphonate moiety (see e.g., WO 97/26270, which is herein incorporated by reference in its entirety).

In some embodiments, the T-Oligo-HES conjugate comprises 3′-cap and/or a 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (See also the stabilizing groups disclosed in Beaucage et al., Tetrahedron 49:1925 (1993); which is herein incorporated by reference in its entirety).

In additional embodiments, the T-Oligo-HES conjugate comprises a 5′-cap and one or more cationic tails. In further embodiments, the oligonucleotide is conjugated with at least 1, 2, 3, 4 or more positively-charged amino acids such as, lysine or arginine. In specific embodiments, the oligonucleotide is a PNA and one or more lysine or arginine residues are conjugated to the C-terminal end of the molecule. In a further preferred embodiment, the oligonucleotide is a PNA and comprises from 1 to 4 lysine and/or arginine residues are conjugated to each PNA linkage.

In additional related embodiments, the disclosure provides T-Oligo-HES conjugates and/or pharmaceutical compositions comprising T-Oligo-HES conjugates that further comprise one or more active agents or therapeutic agents. In one embodiment, the active agent or therapeutic agent is a nucleic acid. In various embodiments, the nucleic acid is a plasmid, an immunostimulatory oligonucleotide, a siRNA, a shRNA, a miRNA, an anti-miRNA, a dicer substrate, a decoy, an aptamer, an antisense oligonucleotide, or a ribozyme.

### Oligonucleotide Synthesis

Oligonucleotides can be synthesized and/or modified by methods well established in the art. Oligomerization of modified and unmodified nucleosides is performed according to literature procedures for DNA-like compounds (Protocols for Oligonucleotides and Analogs, Ed. Agrawal (1993), Humana Press) and/or RNA-like compounds (see, e.g., Scaringe, Methods 23:206-217 (2001) and Gait et al., Applications of Chemically synthesized RNA in RNA: Protein Interactions, Ed. Smith (1998), 1-36. Gallo et al., Tetrahedron 57:5707-5713 (2001), synthesis as appropriate. (see, also, Current Protocols in Nucleic Acid Chemistry, Beaucage, S. L. et al.,(Edrs.), John Wiley & Sons, Inc., New York, N.Y., USA, which is herein incorporated herein by reference in its entirety). Oligonucleotides are preferably chemically synthesized using appropriately protected reagents and a commercially available oligonucleotide synthesizer. Suppliers of oligonucleotide synthesis reagents useful in manufacturing the provided oligonucleotides include, but are not limited to, Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). Alternatively, oligomers may be purchased from various oligonucleotide synthesis companies such as, for example, Dharmacon Research Inc., (Lafayette, Colo.), Qiagen (Germantown, MD), Proligo and Ambion.

In certain embodiments, the preparation of oligonucleotides as disclosed herein is performed according to literature procedures for DNA: Protocols for Oligonucleotides and Analogs, Agrawal, Ed., Humana Press, 1993, and/or RNA: Scaringe, Methods, 23:206-217 (2001); Gait et al., Applications of Chemically synthesized RNA in RNA: Protein Interactions, Smith, Ed., 1998, 1-36; Gallo et al., Tetrahedron 57:5707-5713 (2001). Additional methods for solid-phase synthesis may be found U.S. Pat. Nos. 4,415,732; 4,458,066; 4,500,707; 4,668,777; 4,725,677; 4,973,679; and 5,132,418; and Re. 34,069.

Irrespective of the particular protocol used, the oligonucleotides contained in the provided T-Oligo-HES conjugates can be conveniently and routinely made through the well-known technique of solid phase synthesis. Equipment for such synthesis is sold by several vendors including, for example, Gene Forge (Redwood City, Calif.). Suitable solid phase techniques, including automated synthesis techniques, are described in Oligonucleotides and Analogues, a Practical Approach, F. Eckstein, Ed., Oxford University Press, New York, 1991. Any other means for such synthesis known in the art may additionally or alternatively be employed (including solution phase synthesis).

The synthesis and preparation of the bicyclic sugar modified monomers adenine, cytosine, guanine, 5-methyl-cytosine, thymine and uracil, along with their oligomerization, and nucleic acid recognition properties have been described (Koshkin et al., Tetrahedron, 54:3607-3630 (1998); WO 98/39352 and WO 99/14226), the contents of each of which is herein incorporated by reference in its entirety. Other bicyclic sugar modified nucleoside analogs such as the 4′-CH2—S-2′ analog have also been prepared (Kumar et al., Bioorg. Med. Chem. Lett., 8:2219-2222 (1998)). Preparation of other bicyclic sugar analogs containing oligodeoxyribonucleotide duplexes as substrates for nucleic acid polymerases has also been described (WO 98-DK393 19980914), the contents of each of which is herein incorporated by reference in its entirety

Techniques for linking fluorophores to oligonucleotides such as those used according to the provided methods are well known in the art and can be used or routinely modified to prepare the HES- oligonucleotide complexes contained in the provided T-Oligo-HES conjugate. See, e.g., Connolly et al., Nucleic Acids Res. 13:4485-4502 (1985); Dreyer et al., Proc. Natl. Acad. Sci. 86:9752-9756 (1989); Nelson et al., Nucleic Acids Res. 17:7187-7194 (1989); Sproat et al., Nucleic Acids Res. 15:6181-6196 (1987) and Zuckerman et al., Nucleic Acids Res. 15:5305-5321 (1987), the contents of each of which is herein incorporated by reference in its entirety. Many fluorophores normally contain suitable reactive sites. Alternatively, the fluorophores may be derivatized to provide reactive sites for linkage to another molecule. Fluorophores derivatized with functional groups for coupling to a second molecule are commercially available from a variety of manufacturers. The derivatization may be by a simple substitution of a group on the fluorophore itself, or may be by conjugation to a linker.

Fluorophores are optionally attached to the 5′ and/or 3′ terminal backbone phosphates and/or other bases of the oligonucleotide via a linker. Various suitable linkers are known to those of skill in the art and/or are discussed below. In some embodiments, the linker is a flexible aliphatic linker. In additional embodiments, the linker is a C1 to C30 linear or branched, saturated or unsaturated hydrocarbon chain. In some embodiments, the linker is a C2 to C6 linear or branched, saturated or unsaturated hydrocarbon chain. In additional embodiments, the hydrocarbon chain linker is substituted by one or more heteroatoms, aryls; or lower alkyls, hydroxylalkyls or alkoxys.

In some embodiments, one or more fluorophores are incorporated into an oligonucleotide during automated synthesis using one or more fluoropophore-modified nucleosides, fluorophore and sugar/base/ and/or linkage modified nucleosides, and/or deoxynucleoside phosphoramidites.

In some embodiments, one or more fluorophores are incorporated into an oligonucleotide in a post-synthesis labeling reaction. Appropriate post-synthesis labeling reactions are known in the art and can routinely be applied or modified to synthesize the Oligonucleotide-HES complexes contained in the T-Oligo-HES conjugates provided herein. In one embodiment, one or more fluorophores are incorporated into an oligonucleotide in a post-synthesis labeling reaction in which an amine- or thiol-modified nucleotide or deoxynucleotide in the synthesized oligonucleotide is reacted with an amine- or thiol-reactive fluorophore such as, a succinimidyl ester fluorophore.

In further embodiments, one or more of the same fluorophores are integrated into the oligonucleotide in a single reaction that involves contacting a reactive form of the dye with an oligonucleotide containing a desired number of reactive groups capable of reacting with the fluorophore in a suitable buffer under conditions and for an amount of time sufficient to accomplish the integration of the fluorophores into the oligonucleotide. The reactive groups can routinely be incorporated into the oligonucleotide during synthesis using standard techniques and reagents known in the art.

### Exemplary Modes of Action

In some embodiments, the disclosure provides a method of treating a disease or disorder characterized by the overexpression of a nucleic acid in a subject, comprising administering to the subject an HES-oligonucleotide complex containing an oligonucleotide which is targeted to a nucleic acid comprising or encoding the nucleic acid and which acts to reduce the levels of the nucleic acid and/or interfere with its function in the subject. In further embodiments, the disclosure provides a method of treating a disease or disorder characterized by the overexpression of a protein in a subject, comprising administering to the subject an HES-oligonucleotide complex containing an oligonucleotide which is targeted to a nucleic acid encoding the protein or decreases the endogenous expression, processing or function of the protein in the subject. In some embodiments, the nucleic acid is DNA, mRNA or miRNA. In additional embodiments, the oligonucleotide is selected from a siRNA, shRNA, miRNA, an antagmir (anti-miRNA), a dicer substrate, an antisense oligonucleotide, and a plasmid capable of expressing a siRNA, a miRNA, a ribozyme and an antisense oligonucleotide.

### Antisense

In some embodiments, the T-Oligo-HES conjugate contains an antisense oligonucleotide. The term “antisense oligonucleotide” or simply “antisense” is meant to include oligonucleotides corresponding to single strands of nucleic acids (e.g., DNA, RNA and nucleic acid mimetics such as PNAs morpholinos (e.g., PMOs), and compositions containing modified nucleosides and/or internucleoside linkages) that bind to their cognate mRNA in the cells of the treated subject and modulate RNA function by for example, altering the translocation of target RNA to the site of protein translation, translation of protein from the target RNA, altering splicing of the target RNA (e.g., promoting exon skipping) and altering catalytic activity which may be engaged in or facilitated by the target RNA, and targeting the mRNA for degradation by endogenous RNase H. In some embodiments, the antisense oligonucleotides alter cellular activity by hybridizing specifically with chromosomal DNA. The term antisense oligonucleotide also encompasses antisense oligonucleotides that may not be exactly complementary to the desired target gene. Thus, the provided methods can be utilized in instances where non-target specific-activities are found with antisense, or where an antisense sequence containing one or more mismatches with the target sequence is preferred for a particular use. The overall effect of such interference with target nucleic acid function is modulation of a targeted protein of interest. In the context of the present disclosure, “modulation” means either an increase (stimulation) or a decrease (inhibition) in the expression of a gene or protein in the amount, or levels, of a small non-coding RNA, nucleic acid target, an RNA or protein associated with a small non-coding RNA, or a downstream target of the small non-coding RNA (e.g., a mRNA representing a protein-coding nucleic acid that is regulated by a small non-coding RNA). Inhibition is a suitable form of modulation and small non-coding RNA is a suitable nucleic acid target. Small non-coding RNAs whose levels can be modulated include miRNA and miRNA precursors. In the context of the present disclosure, “modulation of function” means an alteration in the function or activity of the small non-coding RNA or an alteration in the function of any cellular component with which the small non-coding RNA has an association or downstream effect. In one embodiment, modulation of function is an inhibition of the activity of a small non-coding RNA.

Antisense oligonucleotides are preferably from about 8 to about 80 contiguous linked nucleosides in length. In some embodiments, the antisense oligonucleotides are from about 10 to about 50 nucleosides or from about 13 to about 30 nucleotides. Antisense oligonucleotides provided herein include ribozymes, antimiRNAs, external guide sequence (EGS) oligonucleotides (oligozymes), and other short catalytic RNAs or catalytic oligonucleotides which specifically hybridize to the target nucleic acid and modulate its expression.

In some embodiments, the T-Oligo-HES conjugate contains an antisense oligonucleotides from about 15 to about 30 nucleosides in length, (i.e., from 15 to 30 linked nucleosides) or alternatively, from about 17 to about 25 nucleosides in length. In particular embodiments, an antisense oligonucleotide is 8, 9, 10, 11, 12, 13, 14, 15, 16, 17, 18, 19, 20, 21, 22, 23, 24, 25, 26, 27, 28, 29, or 30 nucleosides in length. In additional embodiments, an antisense oligonucleotide is from about 10 to about 50 nucleotides, more preferably about 15 to about 30 nucleotides. In further embodiments, the antisense oligonucleotide is 4, 5, 6 or 7 nucleotides in length.

In additional embodiments, the oligonucleotide in a T-Oligo-HES conjugate interferes with the transcription of a target RNA of interest. In some embodiments, the oligonucleotide interferes with transcription of an mRNA or miRNA of interest by strand displacement. In other embodiments, the oligonucleotide interferes with the transcription of an mRNA by forming a stable complex with a portion of a targeted gene by strand invasion or triplex formation (triplex forming oligonucleotides (THOs), such as those containing LNAs see, e.g., U.S. Appl. Publ. No. 2012/0122104, herein incorporated by reference in its entirety). In additional embodiments, the oligonucleotide in a T-Oligo-HES conjugate interferes with the transcription of a target RNA (e.g., mRNA or miRNA) by interfering with the transcription apparatus of the cell. In some embodiments, the oligonucleotides in the Oligo-HES complex are designed to specifically bind a region in the 5′ end of an mRNA or the AUG start codon (e.g., within 30 nucleotides of the AUG start codon) and to reduce translation. In some embodiments, the oligonucleotide component of the T-Oligo-HES conjugate is designed to specifically hybridize to an intron/exon junction in an RNA. In some embodiments, the oligonucleotide is designed to specifically bind the 3′ untranslated target sequence in an RNA (e.g., mRNA). In further embodiments, the oligonucleotide is designed to specifically bind nucleotides 1-10 of a miRNA. In additional embodiments, the oligonucleotide is designed to specifically bind a sequence in a precursor-miRNA (pre-miRNA) or primary-miRNA (pri-miRNA) that when bound by the oligonucleotide blocks miRNA processing.

In other embodiments, the oligonucleotide in a T-Oligo-HES conjugate binds sites of critical RNA secondary structure or act as steric blockers that cause truncation of the translated polypeptide. In some embodiments, the oligonucleotide is designed to interfere with intron excision, by for example, binding at or near a splice junction of the targeted mRNA. In some embodiments, the oligonucleotide is designed to interfere with intron excision or to increase the expression of an alternative splice variant.

RNase H is an endogenous enzyme that specifically cleaves the RNA moiety of an RNA:DNA duplex. In some embodiments, the antisense oligonucleotides elicit RNase H activity when bound to a target nucleic acid. In some embodiments, the oligonucleotides are DNA or nucleic acid mimetics. Oligonucleotides elicit RNase H activity have particular advantages in for example, harnessing endogenous ribonucleases to reduce targeted RNA.

One antisense design for eliciting RNase H activity is the gapmer motif design in which a chimeric oligonucleotide with a central block composed of DNA, either with or without phosphorothioate modifications, and nuclease resistant 5′ and 3′ flanking blocks, usually 2′-O-methyl RNA but a wide range of 2′ modifications have been used (see Crooke, Curr. Mol. Med., 4(5):465-487 (2004)). Other gapmer designs are described herein or otherwise known in the art.

In additional embodiments, T-Oligo-HES conjugate contains an antisense oligonucleotide that is designed to avoid activation of RNase H in a cell. Oligonucleotides that do not elicit RNase H activity have particular advantages in for example, blocking transcriptional machinery (via a steric block mechanism) and altering splicing of the target RNA. In some embodiments, the oligonucleotides are designed to interfere with and/or alter intron excision, by for example, binding at or near a splice junction of the targeted mRNA. In additional embodiments, the oligonucleotides are designed to increase the expression of an alternative splice variant of a message. In one preferred embodiment, the T-Oligo-HES conjugate contains an oligonucleotide comprising a morpholino (e.g., PMO) antisense oligonucleotide. In another preferred embodiment, the T-Oligo-HES conjugate contains a PNA antisense oligonucleotide.

In particular embodiments, the T-Oligo-HES conjugate contains an antisense oligonucleotide that is targeted to at least a portion of a region up to 50 nucleobases upstream of an intron/exon junction of a target mRNA. More preferably the antisense oligonucleotide is targeted to at least a portion of a region 20-24 or 30-50 nucleobases upstream of an intron/exon junction of a target mRNA and which preferably does not support RNAse H cleavage of the mRNA target upon binding. Preferably, the antisense oligonucleotide contains at least one modification which increases binding affinity for the RNA target (e.g., mRNA and miRNA) and which increases nuclease resistance of the antisense compound.

In one embodiment, the T-Oligo-HES conjugate contains an antisense oligonucleotide comprising at least one nucleoside having a 2′ modification of its sugar moiety. In a further embodiment, the antisense oligonucleotide comprises at least 2, 3, 4, 5, 6, 7, 8, 9, 10, 15, or 20 nucleosides having a 2′ modification of its sugar moiety. In a further embodiment, the antisense oligonucleotide comprises at least 4, 5, 6, 7, 8, 9, 10, 11, 12, 13, 14, 15, 16, 17, 18, 19, 20, 21, 22, 23, 24, 25, 26, 27, 28, 29 or 30 nucleotides having a 2′ modification of its sugar moiety. In yet a further embodiment, every nucleoside of the antisense oligonucleotide has a 2′ modification of its sugar moiety. Preferably, the 2′ modification is 2′-fluoro, 2′-OME, 2′-methoxyethyl (2′-MOE) or a locked nucleic acid (LNA). In some embodiments, the modified nucleoside motif is an LNA or alpha LNA in which a methylene (--CH2--)n group bridges the 2′ oxygen atom and the 4′ carbon atom wherein n is 1 or 2. In further embodiments, the LNA or alpha LNA contains a methyl group at the 5′ position. In some embodiments, the oligonucleotide contains a 2′ modification and at least one internucleoside linkage. In particular embodiment, antisense oligonucleotide contains at least one phosphorothioate internucleoside linkage. In one embodiment, the internucleoside linkages of the oligonucleotide alternate between phosphodiester and phosphorothioate backbone linkages. In another embodiment, every internucleoside linkage of the oligonucleotide is a phosphorothioate linkages.

In additional preferred embodiments, the T-Oligo-HES conjugate contains an antisense oligonucleotide comprising at least one 3′-methylene phosphonate, linkage, LNA, peptide nucleic acid (PNA) linkage or phosphorodiamidate morpholino linkage. In further embodiments, the antisense oligonucleotide contains at least one modified nucleobase. Preferably, the modified nucleobase is a C-5 propyne or 5-methyl C.

In further embodiments, the T-Oligo-HES conjugate contains an antisense oligonucleotide comprising more than 1 or 2 antisense strands that are complementary to different sequences of a target mRNA or a target gene. In some embodiments, the antisense strands are linked linearly or in a branched fashion (e.g., a dendrimer). In further embodiments, the linked antisense strands induce new secondary structures for the target mRNA and /or gene, thereby reducing or inhibiting the appropriate transcription/ translation of targeted nucleotides.

The antisense oligonucleotide compounds contained in the T-Oligo-HES conjugates provided herein can routinely be synthesized using techniques known in the art.

### RNAi - Post Transcriptional Gene Silencing

Short double-stranded RNA molecules and short hairpin RNAs (shRNAs), i.e. fold-back stem-loop structures that give rise to siRNA can induce RNA interference (RNAi). In some embodiments, the T-Oligo-HES conjugate contains an oligonucleotide that induces RNAi. RNAi oligonucleotides contained in the T-Oligo-HES conjugate include, but are not limited to siRNAs, shRNAs and dsRNA DROSHA and/or Dicer substrates. The siRNAs, shRNAs, and one or both strands of the dsRNAs preferably contain one or more modified internucleoside linkages, modified sugar moieties and/or modified nucleobases described herein or otherwise known in the art. These RNAi oligonucleotides have applications including, but not limited to, disrupting the expression of a gene(s) or polynucleotide(s) of interest in a subject. Thus, in some embodiments, the oligonucleotides in the T-Oligo-HES conjugates are used to specifically inhibit the expression of target nucleic acid. In some embodiments, double-stranded RNA-mediated suppression of gene and/or nucleic acid expression is accomplished by administering a T-Oligo-HES conjugate comprising a dsRNA DROSHA substrate, dsRNA Dicer substrate, siRNA or shRNA to a subject and/or cell. Double-stranded RNA-mediated suppression of gene and nucleic acid expression may be accomplished by administering a T-Oligo-HES conjugate comprising a dsRNA, siRNA or shRNA into a subject. SiRNA may be double-stranded RNA, or a hybrid molecule comprising both RNA and DNA, e.g., one RNA strand and one DNA strand.

In some embodiments, the T-Oligo-HES conjugate comprises a siRNA selected from: RNA:RNA hybrids, DNA sense: RNA antisense hybrids, RNA sense: DNA antisense hybrids, and DNA:DNA hybrid duplexes, that are about 21-30 nucleotides long and can associate with a cytoplasmic multi-protein complex known as RNAi-induced silencing complex (RISC). RISC loaded with siRNA mediates the degradation of homologous mRNA transcripts. The provided methods include the use of RNAi molecules comprising any of these different types of double-stranded molecules. In addition, it is understood that RNAi molecules may be used and introduced to cells in a variety of forms. Accordingly, as used herein, RNAi molecules encompass any and all molecules capable of inducing an RNAi response in cells, including, but not limited to, double-stranded polynucleotides comprising two separate strands, i.e. a sense strand and an antisense strand, e.g., small interfering RNA (siRNA); polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

In some embodiments, the T-Oligo-HES conjugate contains oligonucleotides that are double-stranded and 16-30 or 18-25 nucleotides in length. In additional embodiments, a dsRNA oligonucleotide is double-stranded and 17, 18, 19, 20, 21, 22, 23, 24, 25, 26, 27, 28, 29, 30, 31, or 32 nucleotides in length. In particular embodiments, the dsRNA is 21 nucleotides in length. In certain embodiments, the dsRNA 0-7 nucleotide 3′ overhangs or 0-4 nucleotide 5′ overhangs. In particular embodiments, the dsRNA has a two nucleotide 3′ overhang. In a further embodiment, the dsRNA contains two complementary RNA strands of 21 nucleotides in length with two nucleotide 3′ overhangs (i.e., contains a 19 nucleotide complementary region between the sense and antisense strands). In another embodiment, the dsRNA contains two complementary RNA strands of 25 nucleotides in length with two nucleotide 3′ overhangs (i.e., contains a 23 nucleotide complementary region between the sense and antisense strands). In certain embodiments, the overhangs are UU or dTdT 3′ overhangs.

In some embodiments, the T-Oligo-HES conjugate contains a siRNA oligonucleotide that is completely complementary to the corresponding reverse complementary strand of a target RNA. In other embodiments, the siRNA contains 1 or 2 substitutions, deletions or insertions compared to the corresponding reverse complementary strand of a target RNA.

In additional embodiments, the T-Oligo-HES conjugate contains an RNAi oligonucleotide that is a short hairpin RNA. shRNA is a form of hairpin RNA containing a fold-back stem-loop structure that give rise to siRNA and is thus, likewise capable of sequence-specifically reducing expression of a target gene. Short hairpin RNAs are generally more stable and less susceptible to degradation in the cellular environment than siRNAs. The stem loop structure of shRNAs can vary in stem length, typically from 19 to 29 nucleotides in length. In certain embodiments, the T-Oligo-HES conjugate contains a shRNA having a stem that is 19 to 21 or 27 to 29 nucleotides in length. In additional embodiments, the shRNA has a loop size of between 4 to 30 nucleotides in length. While complete complementarity between the portion of the stem that specifically hybridizes to the target mRNA (antisense strand) and the mRNA is preferred, the shRNA may optionally contain mismatches between the two strands of the shRNA hairpin stem. For example, in some embodiments, the shRNA includes one or several G-U pairings in the hairpin stem to stabilize hairpins.

In one embodiment, the nucleic acid target of an RNAi oligonucleotide contained in a T-Oligo-HES conjugate provided herein is selected by scanning the target RNA (e.g., mRNA or miRNA) for the occurrence of AA dinucleotide sequences. Each AA dinucleotide sequence in combination with the 3′ adjacent approximately 19 nucleotides are potential siRNA target sites based off of which an RNAi oligonucleotide can routinely be designed. In some embodiments, the RNAi oligonucleotide target site is not located within the 5′ and 3′ untranslated regions (UTRs) or regions near the start codon (e.g., within approximately 75 bases of the start codon) of the target RNA in order to avoid potential interference of the binding of the siRNP endonuclease complex by proteins that bind regulatory regions of the target RNA.

RNAi oligonucleotide targeting specific polynucleotides can readily be prepared using or routinely modifying reagents and procedures known in the art. Structural characteristics of effective siRNA molecules have been identified. Elshabir et al., Nature 411:494-498 (2001) and Elshabir et al., EMBO 20:6877-6888 (2001). Accordingly, one of skill in the art would understand that a wide variety of different siRNA molecules may be used to target a specific gene or transcript.

### Enzymatic Nucleic Acids

In some embodiments, the T-Oligo-HES conjugates comprise an enzymatic oligonucleotide. Two preferred features of enzymatic oligonucleotides in the provided conjugates are that they have a specific substrate binding site which is complementary to one or more of the target gene DNA or RNA regions, and that they have nucleotide sequences within or surrounding the substrate binding site which impart an RNA cleaving activity to the oligonucleotide. In some embodiments, the enzymatic oligonucleotide is a ribozyme. Ribozymes are RNA-protein complexes having specific catalytic domains that possess endonuclease activity. Exemplary ribozyme oligonucleotides are formed in a hammerhead, hairpin, a hepatitis delta virus, group I intron or RNaseP RNA (in association with an RNA guide sequence) or a Neurospora VS RNA motif.

While the enzymatic oligonucleotides that may be contained in T-Oligo-HES conjugates provided herein may contain modified nucleotides described herein or otherwise known in the art, it is important that such modifications do not lead to conformational changes that abolish catalytic activity of the enzymatic oligonucleotide. Methods of designing, producing, testing and optimizing enzymatic oligonucleotides such as, ribozymes are known in the art and are encompassed herein (see, e.g., WO 91/03162; WO 92/07065; WO 93/15187; WO 93/23569; WO 94/02595, WO 94/13688; EP 92110298; and U.S. Pat. No. 5,334,711, each of which is herein incorporated by reference in its entirety).

### Aptamers and Decoys

In some embodiments, the T-Oligo-HES conjugates contain an aptamer and/or a decoy. As used herein, aptamers refer to a single-stranded nucleic acid molecule (such as DNA or RNA) that assumes a specific, sequence-dependent shape and specifically hybridizes to a target protein with high affinity and specificity. Aptamers in the T-Oligo-HES conjugates are generally fewer than 100 nucleotides, fewer than 75 nucleotides, or fewer than 50 nucleotides in length. The term “aptamer” as used herein, encompasses mirror-image aptamer(s) (high-affinity L-enantiomeric nucleic acids such as, L-ribose or L-2′-deoxyribose units) that confer resistance to enzymatic degradation compared to D-oligonucleotides. In particular embodiments, the T-Oligo-HES conjugate contains the aptamer Macugen (OSI Pharmaceuticals) or ARC1779 (Archemix, Cambridge, Mass.). In additional embodiments, the T-Oligo-HES conjugates provided herein contain an oligonucleotide that competes for target protein binding with the aptamer Macugen (OSI Pharmaceuticals) or ARC1779 (Archemix, Cambridge, Mass.). In additional embodiments, the conjugate contains an oligonucleotide that binds Tat or Rev. In further embodiments, the conjugate contains an oligonucleotide that binds Tat, nucleocapsid, reverse transcriptase, integrase or Rev of HIV-1. In additional embodiments, the conjugate contains an oligonucleotide that binds gp120, HCV NS3 protease, hepatitis C NS3m Yersinia pestis tyrosine phosphatase, intracellular domain of a receptor tyrosine kinases (e.g., EGFRvIII), nucleolin (AML). Methods for making and identifying aptamers are known in the art and can routinely be modified to identify aptamers having desirable diagnostic and/or therapeutic properties and to incorporate these aptamers into the T-Oligo-HES conjugates provided herein. See, e.g., Wlotzka et al., Proc. Natl. Acad. Sci. 99(13):8898-8902 (2002), which is herein incorporated by reference in its entirety.

As used herein, the term “decoy” refers to short double-stranded nucleic acids (including single-stranded nucleic acids designed to “fold back” on themselves) that mimic a site on a nucleic acid to which a factor, such as a protein, binds. Such decoys competitively inhibit and thereby decrease the activity and/or function of the factor. Methods for making and identifying decoys are known in the art and can routinely be modified to identify decoys having desirable diagnostic and/or therapeutic properties, and to incorporate these decoys into the Oligonucleotide-HES complexes of the T-Oligo-HES conjugate. See, e.g., U.S. Pat. No. 5,716,780 which is herein incorporated by reference in its entirety.

### Small Non-Coding RNA and Antagonists (e.g., miRNAs and Anti-miRNAs)

As used herein, the term “small non-coding RNA” is used to encompass, without limitation, a polynucleotide molecule ranging from 17 to 29 nucleotides in length. In one embodiment, a small non-coding RNA is a miRNA (also known as miRNAs, Mirs, miRs, mirs, and mature miRNAs).

MicroRNAs (miRNAs), also known as “mature” miRNA”) are small (approximately 21-24 nucleotides in length), non-coding RNA molecules that have been identified as key regulators of development, cell proliferation, apoptosis and differentiation. Examples of particular developmental processes in which miRNAs participate include stem cell differentiation, neurogenesis, angiogenesis, hematopoiesis, and exocytosis (reviewed by Alvarez-Garcia and Miska, Development, 132:4653-4662 (2005)). miRNA have been found to be aberrantly expressed in disease states, i.e., specific miRNAs are present at higher or lower levels in a diseased cell or tissue as compared to healthy cell or tissue.

miRNAs are believed to originate from long endogenous primary miRNA transcripts (also known as pri-miRNAs, pri-mirs, pri-miRs or pri-pre-miRNAs) that are often hundreds of nucleotides in length (Lee, et al., EMBO J., 21(17):4663-4670 (2002)). One mechanism by which miRNAs regulate gene expression is through binding to the 3′-untranslated regions (3′-UTR) of specific mRNAs. miRNAs nucleotide (nt) RNA molecules that become incorporated into the RNA-induced silencing complex (RISC) mediate down-regulation of gene expression through translational inhibition, transcript cleavage, or both. RISC is also implicated in transcriptional silencing in the nucleus of a wide range of eukaryotes.

In some embodiments, the disclosure provides, inter alia, T-Oligo-HES conjugates and methods for modulating small non-coding RNA activity, including miRNA activity associated with disease states. Certain conjugates and compositions provided herein are particularly suited for use in in vivo methods due to their improved delivery, potent activity and/or improved therapeutic index.

The disclosure provides T-Oligo-HES conjugates and methods for modulating small non-coding RNAs, including miRNA. In particular embodiments, the disclosure provides T-Oligo-HES conjugates and methods for modulating the levels, expression, processing or function of one or a plurality of small non-coding RNAs, such as miRNAs. Thus, in some embodiments, the disclosure encompasses compositions, such as pharmaceutical compositions, comprising a T-Oligo-HES conjugate having at least one oligonucleotide that specifically hybridizes with a small noncoding RNA, such as a miRNA.

In some embodiments, the T-Oligo-HES conjugate contains an oligonucleotide that specifically hybridizes with or sterically interferes with nucleic acid molecules comprising or encoding one or more small non-coding RNAs, such as, miRNAs. In particular embodiments, the disclosure provides T-Oligo-HES conjugates and methods useful for modulating the levels, activity, or function of miRNAs, including those relying on antisense mechanisms and those that are independent of antisense mechanisms.

As used herein, the terms “target nucleic acid,” “target RNA,” “target RNA transcript” or “nucleic acid target” are used to encompass any nucleic acid capable of being targeted including, without limitation, RNA. In one embodiment, the target nucleic acids are non-coding sequences including, but not limited to, miRNAs and miRNA precursors. In a preferred embodiment, the target nucleic acid is a miRNA, which may also be referred to as the miRNA. An oligonucleotide is “targeted to a miRNA” when an oligonucleotide comprises a sequence substantially, including 100% complementary to a miRNA.

As used herein, oligonucleotides are “substantially complementary” to for example, an RNA such as a small non-coding RNA, when they are capable of specifically hybridizing to the small non-coding RNA under physiologic conditions. In some embodiments, an oligonucleotide is “targeted to a miRNA” when an oligonucleotide comprises a sequence substantially, including 100% complementary to at least 8 contiguous nucleotides of a miRNA. In some embodiments, the T-Oligo-HES conjugate contains an oligonucleotide that specifically hybridizes to a miRNA and ranges in length from about 8 to about 21 nucleotides, from about 8 to about 18 nucleotides, or from about 8 to about 14 nucleotides. In additional embodiments, the oligonucleotide specifically hybridizes to a miRNA and ranges in length from about 12 to about 21 nucleotides, from about 12 to about 18 nucleotides, or from about 12 to about 14 nucleotides. In particular embodiments, the oligonucleotide is 8, 9, 10, 11, 12, 13, 14, 15, 16, 17, 18, 19, 20 or 21 monomer subunits (nucleotides) in length. In certain embodiments, the oligonucleotide is 14, 15, 16, 17 or 18 monomer subunits (nucleotides) in length.

In particular embodiments, the T-Oligo-HES conjugate contains an oligonucleotide that has full length complementarity to the miRNA. In other embodiments, the length of complementarity between the oligonucleotide and the target nucleic acid as well as up to 3 “mismatches” between the oligonucleotide and the target miRNA such that the oligonucleotide is still capable of hybridizing with the target miRNA and the function of the oligonucleotide is not substantially impaired. In other embodiments, the oligonucleotide contains a truncation or expansion with respect to the length of target miRNA by up to 6 nucleosides, at either the 3′ or 5′ end, or at both the 3′ and 5′ end of the oligonucleotide. In certain embodiments, the oligonucleotide is truncated by 1 or 2 nucleosides compared with the length of the target miRNA. As a non-limiting example, if the target miRNA is 22 nucleotides in length, the oligonucleotide which has essentially full length complementarity may be 20 or 21 nucleotides in length. In a particular embodiment, the oligonucleotide is truncated by 1 nucleotide on either the 3′ or 5′ end compared to the miRNA.

In some embodiments, the disclosure provides a method of modulating a small non-coding RNA comprising contacting a cell with a T-Oligo-HES conjugate containing an oligonucleotide comprising a sequence that is substantially complementary to the small non-coding RNA, a small non-coding RNA precursor (e.g., a miRNA precursor), or a nucleic acid encoding the small non-coding RNA. As used herein, the term “small non-coding RNA precursor miRNA precursor” is used to encompass any longer nucleic acid sequence from which a small (mature) non-coding RNA is derived and may include, without limitation, primary RNA transcripts, pri-small non-coding RNAs, and pre-small non-coding RNAs. For example, an “miRNA precursor” encompasses any longer nucleic acid sequence from which a miRNA is derived and may include, without limitation, primary RNA transcripts, pri-miRNAs, and pre-miRNAs.

In some embodiments, the disclosure provides, compositions such as pharmaceutical compositions containing a T-Oligo-HES conjugate comprising an oligonucleotide which is targeted to nucleic acids comprising or encoding a small non-coding RNA, and which acts to modulate the levels of the small non-coding RNA, or modulate its function. In further embodiments, the disclosure provides, a composition such as a pharmaceutical composition, containing a T-Oligo-HES conjugate comprising an oligonucleotide which is targeted to a miRNA and which acts to modulate the levels of the miRNA, or interfere with its processing or function.

In some embodiments, the T-Oligo-HES conjugate contains an oligonucleotide that specifically hybridizes to nucleotides 1-10 of a miRNA (i.e., the seed region). In additional embodiments, the oligonucleotide specifically hybridizes to a sequence in a precursor-miRNA (pre-miRNA) or primary-miRNA (pri-miRNA) that when bound by the oligonucleotide blocks miRNA processing.

In additional embodiments, the T-Oligo-HES conjugate contains an oligonucleotide which is targeted to nucleic acids comprising or encoding a small non-coding RNA and which acts to reduce the levels of the small non-coding RNA and/or interfere with its function in a cell.

In other embodiments, the composition contains the T-Oligo-HES conjugate contains an oligonucleotide which comprises or encodes the small non-coding RNA or increases the endogenous expression, processing or function of the small non-coding RNA (e.g., by binding regulatory sequences in the gene encoding the non-coding RNA) and which acts to increase the level of the small non-coding RNA and/or increase its function in a cell.

Oligonucleotides contained in the T-Oligo-HES conjugates provided herein can modulate the levels, expression or function of small non-coding RNAs by hybridizing to a nucleic acid comprising or encoding a small non-coding RNA nucleic acid target resulting in alteration of normal function. For example, non-limiting mechanisms by which the oligonucleotides might decrease the activity (including levels, expression or function) of a small non-coding RNA include facilitating the destruction of the small non-coding RNA through cleavage, sequestration, steric occlusion and by hybridizing to the small non-coding RNA and preventing it from hybridizing to, and regulating the activity of, its normal cellular target(s).

In another embodiment, the disclosure provides a method of inhibiting the activity of a small non-coding RNA, comprising contacting a cell expressing a cell surface antigen with a T-Oligo-HES conjugate comprising a targeting moiety the specifically binds the surface antigen, and an oligonucleotide which is targeted to nucleic acids comprising or encoding a small non-coding RNA and which acts to reduce the levels of the small non-coding RNA and/or interfere with its function in the cell. In some embodiments, the oligonucleotide comprises a sequence substantially complementary nucleic acids comprising or encoding the non-coding RNA. In particular embodiments, the small non-coding RNA is a miRNA.

In an additional embodiment, the disclosure provides a method of inhibiting the activity of a small non-coding RNA, comprising administering to a subject a T-Oligo-HES conjugate containing an oligonucleotide which is targeted to nucleic acids comprising or encoding a small non-coding RNA and which acts to reduce the levels of the small non-coding RNA and/or interfere with its function in the subject. In some embodiments, the targeted nucleic acid is in a cell that expresses a cell surface antigen specifically bound by the targeting moiety of the T-Oligo-HES conjugate. In some embodiments, the targeted nucleic acid is in a cell that is near to a cell that expresses the cell surface antigen that the targeting moiety of the T-Oligo-HES conjugate specifically binds.

In some embodiments, the T-Oligo-HES conjugate contains an oligonucleotide having a sequence substantially complementary nucleic acids comprising or encoding the non-coding RNA. In particular embodiments, the small non-coding RNA is a miRNA.

In an additional embodiment, the disclosure provides a method of increasing the activity of a small non-coding RNA, comprising contacting a cell with a T-Oligo-HES conjugate containing an oligonucleotide which comprises or encodes the small non-coding RNA or increases the endogenous expression, processing or function of the small non-coding RNA (e.g., by binding regulatory sequences in the gene encoding the non-coding RNA) and which acts to increase the level of the small non-coding RNA and/or increase its function in the cell. In some embodiments, the oligonucleotide comprises a sequence substantially the same as nucleic acids comprising or encoding the non-coding RNA. In some embodiments, the oligonucleotide shares 100% identity with at least 15 contiguous nucleotides, at least 20 contiguous nucleotides or over the full-length of the small non-coding RNA sequence. In particular embodiments, the small non-coding RNA is a miRNA.

In another embodiment, the disclosure provides a method of increasing the activity of a small non-coding RNA, comprising administering to a subject in need thereof, a T-Oligo-HES conjugate that contains an oligonucleotide which comprises or encodes the small non-coding RNA or increases the endogenous expression, processing or function of the small non-coding RNA, and which acts to increase the level of the small non-coding RNA and/or increase its function in the subject. In some embodiments, the cell in which the activity of the small non-coding RNA is increased expresses a cell surface antigen specifically bound by the targeting moiety of the T-Oligo-HES conjugate. In some embodiments, the cell in which the activity of the small non-coding RNA is increased is near to a cell that expresses the cell surface antigen that the targeting moiety of the T-Oligo-HES conjugate specifically binds. In some embodiments, the oligonucleotide comprises a sequence substantially the same as nucleic acids comprising or encoding the non-coding RNA. In some embodiments, the oligonucleotide shares 100% identity with at least 15 contiguous nucleotides, at least 20 contiguous nucleotides or over the full-length of the small non-coding RNA sequence. In particular embodiments, the small non-coding RNA is a miRNA.

In additional embodiments, the T-Oligo-HES conjugate contains an oligonucleotide sequence substantially the same as nucleic acids comprising or encoding the small non-coding RNA. In some embodiments, the oligonucleotide is a miRNA mimic. In some embodiments, the miRNA mimic is double stranded. In further embodiments, the oligonucleotide contains a miRNA mimic that is double stranded and contains oligonucleotides of 18-23 units in length and is blunt ended or comprises one or more 3′ overhangs of 1, 2, or 3 nucleotides. In additional embodiments, the oligonucleotide contains a single stranded miRNA mimic that is 18-23 units in length. T-Oligo-HES conjugates containing expression vectors that express these miRNA mimics are also encompassed by the disclosure. In some embodiments, the oligonucleotide shares 100% identity with at least 15 contiguous nucleotides, at least 20 contiguous nucleotides or over the full-length of the small non-coding RNA sequence. In particular embodiments, the small non-coding RNA is a miRNA.

In some embodiments, the disclosure provides a method of treating a disease or disorder characterized by the overexpression of a small-noncoding RNA in a subject, comprising administering to the subject a therapeutically effective amount of a T-Oligo-HES conjugate containing an oligonucleotide which is targeted to nucleic acids comprising or encoding the small non-coding RNA and which acts to reduce the levels of the small non-coding RNA and/or interfere with its function in the subject. In some embodiments, the cell surface antigen specifically bound by the targeting moiety of the conjugate is expressed on the cell surface of the cell in which the activity of nucleic acids is targeted, or on the surface of a nearby cell. In some embodiments, the oligonucleotide is an anti-miRNA (anti-miR). In additional embodiments, the anti-miRNA is double stranded. In further embodiments, the oligonucleotide contains an anti-miRNA that is double stranded and contains oligonucleotides of 18-23 units in length and is blunt ended or comprises one or more 3′ overhangs of 1, 2, or 3 nucleotides. In additional embodiments, the oligonucleotide contains a single stranded anti-miR that is 8-25 units in length. T-Oligo-HES conjugates containing expression vectors that express these anti-MiRs are also encompassed by the disclosure. In some embodiments, the oligonucleotide comprises a sequence substantially complementary to the overexpressed small-noncoding RNA.

In further embodiments, the disclosure provides a method of treating a disease or disorder characterized by the overexpression of a miRNA in a subject, comprising administering to the subject a therapeutically effective amount of a T-Oligo-HES conjugate containing an oligonucleotide which is targeted to nucleic acids comprising or encoding the miRNA and which acts to reduce the levels of the miRNA and/or interfere with its function in the subject. In some embodiments, the cell surface antigen specifically bound by the targeting moiety of the conjugate is expressed on the surface of cells in which the nucleic acid activity is targeted. In some embodiments, the cell surface antigen specifically bound by the targeting moiety of the conjugate is expressed on the surface of cells near the cells in which the nucleic acid activity is targeted. In some embodiments, the oligonucleotide comprises a sequence substantially complementary to the overexpressed miRNA.

Families of miRNAs can be characterized by nucleotide identity at positions 2-8 of the miRNA, a region known as the seed sequence. The members of a miRNA family are herein termed “related miRNAs”. Each member of a miRNA family shares an identical seed sequence that plays an essential role in miRNA targeting and function. As used herein, the term “seed sequence” or “seed region” refers to nucleotides 2 to 9 from the 5′-end of a mature miRNA sequence. Examples of miRNA families are known in the art and include, but are not limited to, the let-7 family (having 9 miRNAs), the miR-15 family (comprising miR-15a, miR-15b, miR15-16, miR-16-1, and miR-195), and the miR-181 family (comprising miR-181a, miR-181b, and miR-181c). In some embodiments, the oligonucleotide in an Oligo-HES complex specifically hybridizes to the seed region of a miRNA and interferes with the processing or function of the miRNA. In some embodiments, the oligonucleotide specifically hybridizes to the seed region of a miRNA and interferes with the processing or function of multiple miRNAs. In further embodiments, at least 2 of the multiple miRNAs have related seed sequences or are members of the miRNA superfamily.

The association of miRNA dysfunction with diseases such as cancer, fibrosis, metabolic disorders and inflammatory disorders and the ability of miRNAs to influence an entire network of genes involved in a common cellular process makes the selective modulation of miRNAs using anti-miRNAs and miRNA mimics particularly attractive disease modulating therapeutics. In some embodiments, the disclosure also provides a method of treating a disease or disorder characterized by the overexpression of a protein in a subject, comprising administering to the subject a T-Oligo-HES conjugate containing an oligonucleotide which is targeted to nucleic acids comprising or encoding a small non-coding RNA that influences the increased production of the protein, wherein the oligonucleotide act to reduce the levels of the small non-coding RNA and/or interfere with its function in the subject. In some embodiments, the cell surface antigen specifically bound by the targeting moiety of the conjugate is expressed on the surface of cells in which the production of the protein is targeted. In some embodiments, the cell surface antigen specifically bound by the targeting moiety of the conjugate is expressed on the surface of cells near the cells in which the production of the protein is targeted. In some embodiments, the oligonucleotide comprises a sequence substantially complementary to the small-noncoding RNA.

In some embodiments, the disclosure provides a method of treating a disease or disorder characterized by the overexpression of a protein in a subject, comprising administering to a subject in need thereof, a T-Oligo-HES conjugate containing an oligonucleotide which is targeted to nucleic acids comprising or encoding a miRNA that influences the increased production of the protein, wherein the oligonucleotide acts to reduce the levels of the miRNA and/or interfere with its function in the subject. In some embodiments, the cell surface antigen specifically bound by the targeting moiety of the conjugate is expressed on the surface of cells in which the production of the protein is targeted. In some embodiments, the cell surface antigen specifically bound by the targeting moiety of the conjugate is expressed on the surface of cells near the cells in which the production of the protein is targeted. In some embodiments, the oligonucleotide comprises a sequence substantially complementary (specifically hybridizable) to the miRNA.

In some embodiments, the disclosure provides a method of treating a disease or disorder characterized by the under expression of a small-noncoding RNA in a subject, comprising administering to the subject a T-Oligo-HES conjugate containing an oligonucleotide which comprises or encodes the small non-coding RNA or increases the endogenous expression, processing or function of the small non-coding RNA, and which acts to increase the level of the small non-coding RNA and/or increase its function in the subject. In some embodiments, the cell surface antigen specifically bound by the targeting moiety of the conjugate is expressed on the surface of cells in which the non-coding RNA is targeted. In some embodiments, the cell surface antigen specifically bound by the targeting moiety of the conjugate is expressed on the surface of cells near the cells in which the non-coding RNA is targeted. In some embodiments, the oligonucleotide comprises a sequence substantially complementary specifically hybridizable) to the overexpressed small-noncoding RNA.

In further embodiments, the disclosure provides a method of treating a disease or disorder characterized by the overexpression of a miRNA in a subject in need thereof, comprising administering to the subject a T-Oligo-HES conjugate containing an oligonucleotide which comprises or encodes the small non-coding RNA or increases the endogenous expression, processing or function of the small non-coding RNA, and which acts to increase the level of the small non-coding RNA and/or increase its function in the subject. In some embodiments, the cell surface antigen specifically bound by the targeting moiety of the conjugate is expressed on the surface of cells in which the non-coding RNA is targeted. In some embodiments, the cell surface antigen specifically bound by the targeting moiety of the conjugate is expressed on the surface of cells near the cells in which the non-coding RNA is targeted. In some embodiments, the oligonucleotide comprises a sequence substantially complementary to the overexpressed miRNA.

In some embodiments, the disclosure provides a method of treating a disease or disorder characterized by the overexpression of a protein in a subject, comprising administering to the subject a T-Oligo-HES conjugate containing an oligonucleotide which comprises or encodes the small non-coding RNA or increases the endogenous expression, processing or function of the small non-coding RNA, and which acts to increase the level of the small non-coding RNA and/or increase its function in the subject. In some embodiments, the cell surface antigen specifically bound by the targeting moiety of the conjugate is expressed on the surface of cells in which the expression of the protein is targeted. In some embodiments, the cell surface antigen specifically bound by the targeting moiety of the conjugate is expressed on the surface of cells near the cells in which the expression of the protein is targeted. In some embodiments, the oligonucleotide comprises a sequence substantially complementary to the small-noncoding RNA.

In some embodiments, the disclosure provides a method of treating a disease or disorder characterized by the overexpression of a protein in a subject, comprising administering to the subject a T-Oligo-HES conjugate containing an oligonucleotide which comprises or encodes the small non-coding RNA or increases the endogenous expression, processing or function of the small non-coding RNA, and which acts to increase the level of the small non-coding RNA and/or increase its function in the subject. In some embodiments, the cell surface antigen specifically bound by the targeting moiety of the conjugate is expressed on the surface of cells in which the expression of the protein is targeted. In some embodiments, the cell surface antigen specifically bound by the targeting moiety of the conjugate is expressed on the surface of cells near the cells in which the expression of the protein is targeted. In some embodiments, the oligonucleotide comprises a sequence substantially complementary (specifically hybridizable) to the miRNA.

In another embodiment, the disclosure provides a method of inhibiting miRNA activity comprising administering to a subject in need thereof T-Oligo-HES conjugate having anti-miRNA activity, such as those described herein. In some embodiments, the cell surface antigen specifically bound by the targeting moiety of the conjugate is expressed on the surface of cells in which the miRNA activity is targeted. In some embodiments, the cell surface antigen specifically bound by the targeting moiety of the conjugate is expressed on the surface of cells near the cells in which the miRNA activity targeted.

In some embodiments, the T-Oligo-HES conjugate contains an oligonucleotide selected from: a siRNA, a miRNA, a dicer substrate (e.g., dsRNA), a ribozyme, a decoy, an aptamer, an antisense oligonucleotide and a plasmid capable of expressing a siRNA, a miRNA, or an antisense oligonucleotide.

In some embodiments, the T-Oligo-HES conjugate contains a chimeric oligonucleotide comprising an internal region containing at least 1, at least 2, at least 3, at least 4, at least 5, or all 2′-F modified nucleotides and external regions comprising at least one stability enhancing modifications. In one embodiment, the oligonucleotide comprises an internal region having a first 2′-modified nucleotide and external regions each comprising a second 2′-modified nucleotide. In a further embodiment, the oligonucleotide contains a gap region that comprises one or more 2′-fluoro modifications and the wing regions comprise one or more 2′-methoxyethyl modifications. In one embodiment, the oligonucleotide in the T-Oligo-HES conjugate is ISIS 393206 or ISIS 327985.

### Targeting Moieties

The term “targeting moiety” is used herein to refer to a molecule that provides the ability to specifically bind a selected target, e.g., a cell surface antigen, a cell, cell type, tissue, organ, region of the body, or a compartment. The targeting moiety can comprise a wide variety of entities and can include naturally occurring molecules, or recombinant or synthetic molecules. In some embodiments, the targeting moiety is an aptamer, avimer, a receptor-binding ligand, a nucleic acid, a biotin-avidin binding pair, a peptide, protein a carbohydrate, lipid, vitamin, a component of a microorganism, a hormone, a receptor ligand (including Fc fusion proteins containing the same), an antibody, an antigen binding portion of an antibody, an alternative binding scaffold, or a derivative of any of the foregoing.

In some embodiments, the targeting moiety is an antibody, an antigen binding portion of an antibody (e.g., a Fv, a Fab, a Fab′, a F(ab′)2, dsFv, Fd, scFv, and diabodies), or a single-domain antibody. In some embodiments, the targeting moiety is a monoclonal antibody, human antibody (e.g., a full-length human antibody), a humanized antibody, a bi-specific antibody, a multispecific antibody, a synthetic antibody, or a pegylated antibody. In some embodiments the targeting moiety is a full length IgG1, IgG2, or IgG4 antibody.

In additional embodiments, the targeting moiety is an alternative binding scaffold molecule. In some embodiments, the targeting moiety is an alternative binding scaffold selected from: an affibody, nanobody (VHH), VNAR, anticalin, fynomer, DARPin, Tetranectin, Transbody, AdNectin, Affilin, Microbody, peptide aptamer, alterase, plastic antibody, phylomer, stradobody, maxibody, evibody, Z domain, D domain, armadillo repeat protein, Kunitz domain, avimer, atrimer, probody, immunobody, triomab, troybody, pepbody, vaccibody, UniBody, Affimer, and a DuoBody.

In various embodiments, the targeting moiety of the T-Oligo-HES conjugate is an antigen binding portion (fragment) of an antibody (e.g., a Fab, scFv and a single domain antibody) capable of specifically binding to a cell surface antigen. Antibody fragments, such as Fab and scFv, have advantages over full-length antibodies as they are less bulky and may lack an Fc domain, which may interfere with in vivo delivery.

In some embodiments the targeting moiety of the T-Oligo-HES conjugate is an activable antibody. Activable antibodies are protease-activated antibodies designed to improve the targeting selectivity to the region of disease sites (i.e., to target antibody) by masking the binding sites of antibodies with inhibitory domains that are cleaved and removed by proteases that are highly expressed at the disease sites. In some embodiments, the targeting moiety of the T-Oligo-HES conjugate is an activable antibody and the antibody is conjugaged to at least one Oligo-HES complex by a cleavable ligand. In some embodiments, the mask of the binding site of the activable antibody is cleaved by the same protease as at least one cleavable linker that conjugates the antibody to an Oligo-HES complex in the conjugate. In some embodiments, the mask of the antigen binding site of the activable antibody is cleaved by a different protease compared to at least one cleavable linker that conjugates the antibody to an Oligo-HES complex in the conjugate. For example, in some embodiments, the mask of the antigen binding site of the activable antibody (e.g., an IgG antibody) is cleaved by a protease that is highly expressed at a disease site (e.g., a tumor) and at least one cleavable linker in the conjugate that conjugates the antibody to an Oligo-HES complex is cleaved by an enzyme of the immune complement system (e.g., u-plasminogen activator, tissue plasminogen activator, trypsin, or plasmin. Activable antibodies are described in Intl. Appl. Publ. No. WO 2016/179285, the contents of which is herein incorporated by references in its entirety and for all purposes).

In some embodiments the targeting moiety of the T-Oligo-HES conjugate is a therapeutic antibody. In some embodiments the term “therapeutic antibody” refers to an antibody that binds to a therapeutic target molecule and is expected to result in alleviation, or a decrease in the progression, of a disease in vivo. In some embodiments, the therapeutic antibody specifically binds a cell surface antigen. In further embodiments, the term “therapeutic antibody” denotes an antibody which is being or has been tested in clinical studies for approval as human therapeutic and which can be administered to an individual for the treatment of a disease. In yet further embodiments, the “therapeutic antibody” has been approved for administration as a human therapeutic by at least one regulatory agency. In additional embodiments, the therapeutic antibody is an antibody that has been approved further embodiments, the targeting moiety is a therapeutic antibody selected from: trastuzumab (HER2/neu), pertuzumab (HER2/neu), panitumumab (EGFR), nimotuzumab (EGFR), zalutumumab (EGFR), cetuximab (EGFR), (HER3), onartuzumab (c-MET), patritumab, clivatuzumab (MUC1), sofituzumab (MUC16), edrecolomab, (EPCAM), adecatumumab (EPCAM), anetumab (MSLN), huDS6 (CA6), lifastuzumab (NAPI2B), sacituzumab (TROP2), PR1A3, humanized PR1A3 (CEA), humanized Ab 2-3 (CEA), IMAB362/claudiximab (Claudin18.2), AMG595 (EGFRvIII), ABT806 (EGFRvIII), sibrotuzumab (FAP), DS-8895a variant 1 (EphA2), DS-8895a variant 2 (EphA2), anti-EphA2 (EphA2), MEDI-547 (EphA2), narnatumab (RON), RG7841 (LY6E), farletuzumab (FRA /folate receptor alpha), mirvetuximab (FRA), J591 variant 1 (PSMA), J591 variant 2 (PSMA), rovalpituzumab (DLL3), PF-06647020 (PTK7), anti-PTK7 (PTK7), ladiratuzumab (LIV1), cirmtuzumab (ROR1), rituximab (CD20), ibritumomab tiuxetan (CD52), alemtuzumab (CD33), gemtuzumab ozogamicin (CD33), CT-011 (PD1), tositumomab (CD20), ipilimumab (CTLA4), tremelimumab (CP-675,206)(CTLA4), nivolumab (PD1), and pembrolizumab (PD1), durvalumab (PDL1) anti-MAGE-A3, or anti-NY-ESO-1.

In particular embodiments, the targeting moiety of the T-Oligo-HES conjugate is the therapeutic antibody trastuzumab.

In particular embodiments, the targeting moiety of the T-Oligo-HES conjugate is the therapeutic antibody cetuximab.

In particular embodiments, the targeting moiety of the T-Oligo-HES conjugate is the therapeutic antibody rituximab.

In particular embodiments, the targeting moiety of the T-Oligo-HES conjugate is the therapeutic antibody bevacizumab (VEGF).

In particular embodiments, the targeting moiety of the T-Oligo-HES conjugate is the therapeutic antibody girentuximab (CAIX).

In particular embodiments, the targeting moiety of the T-Oligo-HES conjugate is the therapeutic antibody nivolumab.

In particular embodiments, the targeting moiety of the T-Oligo-HES conjugate is the therapeutic antibody pembrolizumab.

In some embodiments, the target of interest specifically bound by the targeting moiety is a cell surface antigen on or near a cell or tissue of interest. In particular embodiments, the cell or tissue of interest is a diseased cell, a cancer cell, an immune cell, an infected cell, or an infectious agent. In some embodiment, the target of interest specifically bound by the targeting moiety is a disease-related antigen. In some embodiments the targeting moiety specifically binds a cell surface antigen characteristic of a cancer, and/or of a particular cell type (e.g., a hyperproliferative cell). In some embodiments the targeting moiety specifically binds a cell surface antigen associated with a disorder of the immune system.

In some embodiments, the target of interest specifically bound by the targeting moiety is a surface antigen expressed on an infected cell. In some embodiments, the targeting moiety specifically binds an infectious agent such as pathogen (e.g., a bacterial cell such as tuberculosis, smallpox, and anthrax; a virus such as HIV; a parasite such as malaria and leishmaniosis; a fungal infection; a mold; and a mycoplasma). In an additional embodiment, the target of interest bound by the targeting moiety is a bacterial antigen, a viral antigen, a fungal antigen, a mycoplasm antigen, a prion antigen, or a parasite antigen (e.g., one infecting a mammal). In one embodiment, the target of the Targeting Moiety is anthrax, hepatitis b, rabies, Nipah virus, west Nile virus, a meningitis virus, or CMV. In an additional embodiment, the Targeting Moiety specifically binds a pathogen.

In some embodiments, the targeting moiety specifically binds a cell surface antigen that does not internalize the conjugate upon binding. In other embodiments, the targeting moiety specifically binds a cell surface antigen that internalizes the conjugate upon binding. In some embodiments, the targeting moiety specifically binds a cell surface antigen(s) derived, from or determined to be expressed on, a specific subject’s cancer (e.g., tumor) such as a neoantigen.

In some embodiments, the targeting moiety specifically binds a tumor cell surface antigen. The term “tumor cell surface antigen” (TSA) refers to an antigen that is common to a specific hyperproliferative disorder such as cancer. In some embodiments, the targeting moiety specifically binds a tumor cell surface antigen that is a tumor associated antigen (TAA). A TAA is an antigen that is found on both tumor and some normal cells. A TAA may be expressed on normal cells during fetal development when the immune system is immature and unable to respond or may be normally present at extremely low levels on normal cells but which are expressed at much higher levels on tumor cells. Because of the dynamic nature of tumors, in some instances, tumor cells may express unique antigens at certain stages, and at others also express antigens that are also expressed on non-tumor cells. Thus, inclusion of a certain marker as a TAA does not preclude it being considered a tumor specific antigen. In some embodiments, the targeting moiety specifically binds a tumor cell surface antigen that is a tumor specific antigen (TSA). A TSA is an antigen that is unique to tumor cells and does not occur on other cells in the body. In some embodiments, the targeting moiety specifically binds a tumor cell surface antigen expressed on the surface of a cancer including but not limited to primary or metastatic melanoma, thymoma, lymphoma, sarcoma, lung cancer (e.g., NSCLC or SCLC), liver cancer, non-Hodgkin’s lymphoma, Hodgkin’s lymphoma, leukemias, multiple myeloma, glioblastoma, neuroblastoma, uterine cancer, cervical cancer, renal cancer, thyroid cancer, bladder cancer, kidney cancer, mesothelioma, and adenocarcinomas such as breast cancer, prostate cancer, ovarian cancer, pancreatic cancer, colon cancer and other cancers known in the art. In some embodiments, the targeting moiety specifically binds cell surface antigen expressed on the surface of a cell in the tumor microenvironment (e.g., and antigen such as VEGFR and TIE1, or TIE2 expressed on endothelial cells and macrophage, respectively, or an antigen expressed on tumor stromal cells such as cancer-associated fibroblasts (CAFs) tumor infiltrating T cells and other leukocytes, and myeloid cells including mast cells, eosinophils, and tumor-associated macrophages (TAM).

In some embodiments, the targeting moiety specifically binds a tumor cell surface antigen on a leukemic cell, lymphoma cell, pancreatic cancer cell, breast cancer cell, melanoma cell, lung cancer cell, head and neck cancer cell, ovarian cancer cell, bladder cancer cell, colon cancer cell, kidney cancer cell, liver cancer cell, prostate cancer cell, bone cancer cell, or brain cancer cell including a glioblastoma cell; or any lymphoma, myeloma, blastoma, sarcoma, leukemia or carcinoma cell.

In some embodiments, the targeting moiety specifically binds a cell surface antigen selected from: CD5, CD19, CD20, CD25, CD37, CD30, CD33, CD45, CD204, CD206, CD301, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Ley, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, HER2/neu, MAGE A3, P53 nonmutant, NY-ESO-1, MelanA/MART1, Ras mutant, gp100, P53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PDL, VTCN1, and VISTA.

In additional embodiments, the targeting moiety specifically binds a cell surface antigen selected from: HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15.

In some embodiments, the targeting moiety specifically binds a cell surface antigen that internalized conjugate after binding. In some embodiments, the targeting moiety specifically binds a cell surface antigen selected from: GONMB, TACSTD2 (TROP2), CEACAMS, EPCAM, a folate receptor (e.g., folate receptor-.alpha., folate receptor-.beta. or folate receptor-.delta.), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), RAAG12, 5T4 (TPBG), SLTRK6, SC-16, Tissue factor, LIV-1 (ZIP6), CGEN-15027, P Cadherin, Fibronectin Extra-domain B (ED-B), VEGFR2 (CD309), Tenascin, Collagen IV, Periostin, endothelin receptor, HER2, HER3, ErbB4, EGFR, EGFRvIII, FGFR1, FGFR2, FGFR3, FGFR4, FGFR6, IGFR-1, FZD1, FZD2, FZD3, FZD4, FZD5, FZD6, FZD7, FZD8, FZD9, FZD10, SMO, CD2, CD3, CD4, CD5, CD6, CD8, CD11, CD11a, CD15, CD18, CD19, CD20, CD22, CD26, CD27L, CD28, CD30, CD33, CD34, CD37, CD38, CD40, CD44, CD56, CD70, CD74, CD79, CD79b, CD98, CD105, CD133, CD138, cripto, IGF-1R, IGF-2R, EphA1 an EphA receptor, an EphB receptor, EphA2, EphA3, EphA4, EphA5, EphA6, EphA7, EphA8, EphA1, EphB1, EphB2, EphB3, EphB4, EphB6, an integrin (e.g., integrin avb3, avb5, or avb6), a C242 antigen, Apo2, PSGR, NGEP, PSCA, TMEFF2, endoglin, PSMA, CanAg, CALLA, c-Met, VEGFR-1, VEGFR-2, DDR1, PDGFR alpha., PDGFR beta, TrkA, TrkB, TrkC, UFO, LTK, ALK, Tie1, Tie2, PTK7, Ryk, TCR, NMDAR, LNGFR, and MuSK.

In some embodiments, the targeting moiety specifically binds a tumor microenvironment cell surface antigen (including certain membrane anchored proteases). In further embodiments, the targeting moiety specifically binds a cell surface antigen expressed on endothelial cells or macrophages (e.g., VEGFR, TIE1, and TIE2), or tumor stromal cells such as cancer-associated fibroblasts (CAFs), and tumor infiltrating T cells and other leukocytes, and myeloid cells including mast cells, eosinophils, and tumor-associated macrophages.

In some embodiments, the targeting moiety specifically binds a cell surface antigen selected from: PD1, PD-L1, PD-L2, CTLA4 LAG3, TIM-3, TIGIT, VISTA, B7-H3, BTLA, A2aR and CD73.

In additional embodiments, the targeting moiety specifically binds a cell surface antigen on an immune cell. In some embodiments, the immune cell is diseased, activated, leukemic or normal. In further embodiments, the targeting moiety specifically binds a cell surface antigen expressed on one or more immune cells, which can include, without limitation, a cell of lymphoid of myeloid origin, a T cell, cytotoxic T lymphocyte, T helper cell, natural killer (NK) cell, natural killer T (NKT) cell, an anti-tumor macrophage (e.g., M1 macrophages), a B cell, a dendritic cell, or a subset thereof. In some embodiments the targeting moiety specifically binds a cell surface antigen expressed on an immune cell of lymphoid or myeloid origin such as a T cell, a B cell, an NK cell, an NKT cell, or a dendritic cell. In some embodiments, the cell surface antigen is found on a megakaryocyte, thrombocyte, erythrocyte, mast cell, basophil, neutrophil, eosinophil, or a subset thereof.

In further embodiments, the targeting moiety specifically binds a cell surface antigen on an antigen presenting cell. In some embodiments, the targeting moiety specifically binds an antigen selected from: OX40L, 4-1BBL, MARCO, DC-SIGN, Dectin1, Dectin2, DEC-205, CLEC5A, CLEC9A, CLEC10A, CLEC12A, CD1A, CD16A, CD32A, CD32B, CD36, CD40, CD47, CD64, CD204, CD206, HVEM, PDL1, mannose scavenger receptor1, and BDCA2.

### Linkers

In some embodiments, the targeted Oligo-HES conjugate comprises one or more linkers.

In some embodiments, the T-Oligo-HES conjugate comprises a linker connecting the targeting moiety and an oligonucleotide of the Oligo-HES complex. In some embodiments, the linker connects 1-30, 1-20, 1-10, or 1-5 Oligo-HES complexes to the targeting moiety of the T-Oligo-HES complex. In some embodiments, the T-Oligo-HES conjugate comprises a plurality of linkers connecting oligonucleotides to the targeting moiety. In some embodiments, the T-Oligo-HES conjugate comprises 1, 2, 3, 4, 5, 6, 7, 8, 9, 10, 1-10, 1-15, or 1-20, linkers connecting oligonucleotides to the targeting moiety. In some embodiments, the T-Oligo-HES conjugate comprises 1-30, 1-20, 1-10, or 1-5 linkers connecting oligonucleotides to the targeting moiety. In some embodiments, 1, 2, 3, 4, or more of the linkers are different. In some embodiments, 1, 2, 3, 4, or more, or all of the linkers are the same. In some embodiments, 1, 2, 3, 4, or more, or all, of the linkers that connect the oligonucleotides to the targeting moiety in the T-Oligo-HES conjugate are the same. In some embodiments, 1, 2, 3, 4, or more, of the linkers that connect the oligonucleotides to the targeting moiety in the T-Oligo-HES conjugate are the different.

In additional embodiments, the T-Oligo-HES conjugate comprises a linker between oligonucleotides in an Oligo-HES complex contained in the T-Oligo-HES conjugate. In some embodiments, the T-Oligo-HES conjugate comprises a plurality of linkers between oligonucleotides in the Oligo-HES complexes of the conjugate. In some embodiments, the T-Oligo-HES conjugate comprises 1, 2, 3, 4, 5, 6, 7, 8, 9, 10, 1-10, 1-15, or 1-20, linkers between oligonucleotides in the Oligo-HES complexes of the conjugate. In some embodiments, 1, 2, 3, 4, or more, or all, of the linkers between oligonucleotides in the Oligo-HES complexes of the conjugate are the same. In some embodiments, 1, 2, 3, 4, or more of the linkers between oligonucleotides in the Oligo-HES complexes of the conjugate are different.

In additional embodiments, the T-Oligo-HES conjugate comprises a linker connecting the targeting moiety and an oligonucleotide of conjugate and the conjugate also comprises a linker between oligonucleotides in an Oligo-HES complex of the conjugate. In further embodiments, the T-Oligo-HES conjugate comprises a plurality of a linkers connecting the targeting moiety and the oligonucleotide of conjugate and a plurality of linkers between oligonucleotides in an Oligo-HES complex of the conjugate.

In some embodiments, vectors encoding the provided targeted moiety of the Oligo-HES conjugate linked as a single nucleotide sequence to any of the linkers described herein are provided and may be used to prepare such targeting moiety-linker complexes.

In some embodiments, the linker length allows for efficient binding of the targeting moiety and T-Oligo-HES conjugate to a cell surface antigen of interest (e.g., a cancer surface antigen, immune cell surface antigen, or infectious agent surface antigen as described herein. Preferably the linker length optimizes the spacing between targeting moiety and the Oligo-HES complexes in the T-Oligo-HES conjugate, and to otherwise optimize linker functionality.

The T-Oligo-HES conjugates can contain a variety of linker sequences and each T-Oligo-HES conjugate can comprise different linkers containing different compositions. For example, the linker connecting oligonucleotides to the targeting moiety of the conjugate may be a linear peptide of 15 to 30 amino acid residues in length, whereas a linker between oligonucleotides in an Oligo-HES complex of the conjugate may be linear or branched alkyl such as a C2-6, C10, or C18 linear alkyl).

In various embodiments, the linker may be derived from naturally-occurring multi-domain proteins or are empirical linkers as described, for example, in Chichili et al., Protein Sci. 22(2):153-167 (2013), Chen et al., Adv Drug Deliv Rev. 65(10):1357-1369 (2013), the entire contents of which are hereby incorporated by reference. In some embodiments, the linker is designed using linker designing databases and computer programs such as those described in Chen et al., Adv Drug Deliv Rev. 65(10): 1357-1369 (2013), and Crasto et al., Protein Eng. 13(5):309-312 (2000), the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the T-Oligo-HES conjugate.

The linker(s) may be linear or branched. In particular embodiments, hetero- bifunctional cross-linkers are used that eliminate unwanted homopolymer formation.

In some embodiments, the T-Oligo-HES conjugate comprises a peptide linker (e.g., a peptide linker connecting the targeting moiety and an oligonucleotide of the Oligo-HES complex). In some embodiments, the linker is less than 50 amino acid residues long. For example, the peptide linker may be less than 50, 45, 40, 35, 30, 25, 20, 19, 18, 17, 16, 15, 14, 13, 12, 11, 10, 9, 8, 7, 6, 5, 4, or 3 amino acid residues in length. In some embodiments, the peptide linker is 2 to 50, 2 to 40, 2 to 30, or 2 to 20, 2 to 15, or 2 to 10 amino acid residues in length. In some embodiments, the peptide linker is up to 15 or up to 30 amino acid residues in length. In some embodiments, the peptide linker is 5 to 50, 10 to 40, or 15 to 30 amino acid residues in length. In further embodiments, the linker is a linear peptide (e.g., a linear peptide of up to 15, up to 30, 5 to 50, 10 to 40, or 15 to 30 amino acid residues in length).

In some embodiments, the T-Oligo-HES conjugate comprises a protease substrate - therapeutic oligo conjugate linking arm (Cargo-Projection Arm) linker arm between the targeting ligand (e.g., antibody) and protease substrate that provides a rigid with a rigid linker are arm. In some embodiments, the linker arm comprises the sequence 5’-CUCUCCTTCTAGCCTCCGCTAGTCAAAAUU -3’ (SEQ ID NO: 80). In further embodiments, the linker arm comprises the sequence 5’ - [C6 Amino Linker Arm] -CUCUCCTTCTAGCCTCCGCTAGTCAAAAUU[C6 AMINO LINKER ARM]-3′ (SEQ ID NO: 80). In further embodiments, the nucleotides of the linker arm are comprises the sequence 5’ - [C6 Amino Linker Arm] - CUCUCCTTCTAGCCTCCGCTAGTCAAAAUU[C6 AMINO LINKER ARM]-3′ (SEQ ID NO: 80), wherein the is a posphorothioate backbone in each linkage of the oligo. In further embodiments, the nucleotides of the linker arm are comprises the sequence 5’ - [C6 Amino Linker Arm] - CUCUCCTTCTAGCCTCCGCTA GTCAA^{x}A^{x}A^{x}U^{x}U^{x} [C6 AMINO LINKER ARM]-3′ (SEQ ID NO: 80), wherein the is a posphorothioate backbone in each linkage of the oligo and wherein ^{x} denotes a 2’OMe modification on 2’ OH group of the sugar. In some embodiments, the oligo linker arm further comprises a sense strand having the sequence 5’- ATTTTGACTAGCGGA GGCTAGAAGGAGA -3’ (SEQ ID NO: 81). While not wishing to be bound by theory, the presence of this sense strand to the linker arm, i.e. Cargo-Projection Arm between antibody and protease substrate, is thought to add to add to the rigidity of the arm and thereboy project the protease substrate out toward the target cells membrane so that cell surface expressed proteases can recognize the substrate attached to targeting ligand (e.g., antibody) and cleave. Thereby releasing the therapeutic oligonucleotide carrying half of the protease substrate peptide off the antibody.

In some embodiments, the T-Oligo-HES conjugate comprises a linker that is cleavable. In some embodiments, the linkers of the conjugate are non-cleavable. In some embodiments, the T-Oligo-HES conjugate comprises one or more linkers that are cleavable and one or more linkers that are non-cleavable.

In particular embodiments, the T-Oligo-HES conjugate contains a cleavable linker comprising an amino acid sequence that is a substrate for at least one protease such as an extracellular protease. In some embodiments, the cleavable linker has an amino acid sequence comprising a protease cleavage site containing P1-P1′ residues and having a looped conformation. Suitable an amino acid sequence that are substrates for proteases of interest can routinely be identified using any of a variety of known techniques. For example, peptide substrates can be identified using the methods described in U.S. Pat. Nos. 7,666,817, 8,563,269, and Intl. Publ. No. WO 2014/026136, the contents of each of which is hereby incorporated by reference in their entirety. (See, also, Boulware et al. Biotechnol Bioeng. 106(3): 339-346 (2010)).

In particular embodiments, the linker is a peptide comprising a conformation dependent cleavage site. It is believed that this conformation dependent cleavage protease substrate provides a defined secondary structure which results in increased speicifity and sensitivity compared to linear sequence protease cleavange sites. The latter allow the cleavage of the substrate with a little amount of the target protease.

In further particular embodiments, the linker includes a Cargo-Projection Arm, as further described herein that is rigid and is thought to physically expose the protease substrate outward from the antibody toward the target cell surface where the protease is localized.

In particular embodiments, the T-Oligo-HES conjugate contains a cleavable linker comprising an amino acid sequence that is a substrate for at least one protease that is active in a diseased tissue. For example, in some embodiments, the T-Oligo-HES conjugate contains a cleavable linker comprising an amino acid sequence that is a substrate for at least one protease that is active and/or colocalized with cells that express a cell surface antigen specifically bound by the targeting moiety of the T-Oligo-HES conjugate (e.g., in a tumor and/or tumor microenvironment). In some embodiments, the cleavable linker comprises an amino acid sequence that is a substrate for at least one protease that is or is believed to be up-regulated in inflammation. In some embodiments, the cleavable linker comprises an amino acid sequence that is a substrate for at least one protease that is or is believed to be up-regulated or otherwise unregulated in autoimmunity.

In some embodiments, the cleavable linker comprises an amino acid sequence that is a substrate for at least one protease that is or is believed to be up-regulated or otherwise unregulated in cancer. In particular embodiments, the T-Oligo-HES conjugate contains a cleavable linker comprising an amino acid sequence that is a substrate for at least one protease selected from: a metalloproteinase (e.g., meprin, neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a Type II Transmembrane Serine Protease (TTSP) such, as DESC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

In some embodiments, the cleavable linker comprises an amino acid sequence that is a substrate for at least one protease selected from: hepsin (HPN), furin, matriptase, matriptase-2, a gelatinase (e.g., gelatinase A (MMP 2), and progelatinase B (MMP 9) and progelatinase A), TMPRSS2, TMPRSS3, TMPRSS4 (CAP2), fibroblast activation protein (FAP), kallikrein-related peptidase (KLK family), KLK4, KLK5, KLK6, KLK7, KLK8, KLK10, KLK11, KLK13, and KLK14.

In some embodiments, the cleavable linker comprises an amino acid sequence that is a substrate for at least one ADAM with thrombospondin motifs (ADAMTS) selected from ADAMTS1, ADAMTS4, and ADAMTS5.

In some embodiments, the T-Oligo-HES conjugate contains a cleavable linker comprising an amino acid sequence that is a substrate for at least one protease selected from: (a) MMP9; (b) MMP14; (c) MMP1, MMP2 (e.g., gelatinase A), MMP3, MMP7, MMP8, MMP10, MMP11, MMP12, MMP13, MMP15, MMP16, MMP17, MMP19, MMP20, MMP23, MMP24, MMP26, and MMP27; (d) a serine protease (e.g., MT-SP1 and uPA); (e) a cysteine protease; (f) a metalloprotease including (a)-(c); (g) an aspartyl protease; and (h) a threonine protease. In some embodiments, the T-Oligo-HES conjugate contains a cleavable linker comprising an amino acid sequence that is a substrate for at least one MMMP. In some embodiments, the cleavable linker comprises an amino acid sequence that is a substrate for MMP9 or MMP14.

In some embodiments, the T-Oligo-HES conjugate contains a cleavable linker comprising an amino acid sequence that is a substrate for at least one enzyme or protease selected from: an ADAMS/ADAMTS, (e.g., ADAM8, ADAM9, ADAM 10, ADAM 12, ADAM 15, ADAM 17/TACE, ADAMDEC1, ADAMTS1, ADAMTS4, ADAMTS5); Cathepsin E); a caspase (e.g., Caspase 1-10, and Caspase 14); a cysteine cathepsin (e.g., Cathepsin B, Cathepsin K, Cathepsin L, Cathepsin S, Cathepsin V/L2, and Cathepsin X/Z/P); a KLK (e.g., KLK4-8, KLK10, KLK 11, KLK 13, and KLK14); a metalloproteinase (e.g., Meprin, Neprilysin, PSMA, and BMP1); Cathepsin A, Chymase, a coagulation factor protease (e.g., FVIIa, FIXa, FXa, FXIa, and FXIIa); Granzyme B; Guanidinobenzoatase; HtrAl; lactoferrin; Marapsin; NS3/4A; PACE4; Plasmin; PSA, tPA; tryptase; DPP-4; and hepsin.

Suitable amino acid sequences for substrates containing cleavage sites for proteases are known in the art and can be included in the sequence of the cleavable linkers provided herein. In some embodiments, the T-Oligo-HES conjugate contains a cleavable linker comprising an amino acid sequence of a protease substrate disclosed in Intl. Appl. Publ. No. WO2019018828A1 (see, e.g., paragraphs 105-117, and Table 4, Table 6, page 68, and the substrate sequences corresponding to each of sequence indentifer NOS:393 (356-423, 680-698, 713, 714, 789-808, and 1037), and WO 2016/179285 (see, e.g., pages 40-47), the contents of each of which is herein incorporated by references in its entiretiy and for all purposes.

In some embodiments, the T-Oligo-HES conjugate contains a linker cleaved by an enzyme of the immune complement system (complement cascade), such as but not limited to u-plasminogen activator, tissue plasminogen activator, trypsin, and plasmin. The targeting moiety of these conjugates preferably contains an antibody that can activate complement and the antibody in the conjugate retains both the ability to bind antigen and to activate the complement cascade. According to one embodiments, an Oligo-HES complex is conjugated to the antibody via a linker susceptible to cleavage by complement. Thus, when these conjugates bind to antigen in the presence of complement, the linker is cleaved and the Oligo-HES complex is released from the conjugate. The T-Oligo-HES conjugate thus, activates the complement cascade and releases the Oligo-HES complex at the target site. In another embodiment the Oligo-HES complex is attached/conjugated via a linker susceptible to cleavage by enzymes having a proteolytic activity such as a u- plasminogen activator, a tissue plasminogen activator, plasmin, or trypsin.

In some embodiments, the T-Oligo-HES conjugate contains a linker that is cleavable by a cleaving agent that is present in the intracellular environment (e.g., within a lysosome or endosome or caveolae). The linker can be, e.g., a peptidyl linker that is cleaved by an intracellular peptidase or protease enzyme, including, but not limited to, a lysosomal or endosomal protease. In some embodiments, the linker is a peptide of at least 2 or 3 amino acids residues in length. Cleaving agents can include cathepsins B and D and plasmin, all of which are known to hydrolyze dipeptide drug derivatives resulting in the release of active drug inside target cells (see, e.g., Dubowchik and Walker, Pharm. Therapeutics 83:67-123 (1999)). Most typical are peptidyl linkers. In particular embodiments, the peptide linker is cleavable by cathepsin-B, which is highly expressed in cancerous tissue. In some embodiments, the linker contains the sequence Phe-Leu or Gly-Phe-Leu-Gly (SEQ ID NO: 9), or a cathepsin-B cleavable amino acid sequence disclosed in U.S. Pat. No. 6,214,345, incorporated herein. In a specific embodiment, the linker is a Val-Cit linker or a Phe-Lys linker (see, e.g., U.S. Pat. No. 6,214,345).

In some embodiments, the T-Oligo-HES conjugate contains a pH-sensitive cleavable linker, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123 (1999); and Neville et al., Biol. Chem. 264: 14653-14661 (1989)). Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the targeting moiety via an acylhydrazone bond (see, e.g., U.S. Pat. No. 5,622,929).

In some embodiments, the T-Oligo-HES conjugate contains a linker that is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alphamethyl-alpha-(2-pyridyl-dithio)toluene)-, SPDB and SMPT. (See, e.g., Thorpe et al., Cancer Res. 47:5924-5931 (1987); Wawrzynczak et al., In Immunoconjugates: Antibody Conjugates in Radioimagery and Therapy of Cancer (C. W. Vogel ed., Oxford U. Press, 1987). See also U.S. Pat. No. 4,880,935.)

In other embodiments, the T-Oligo-HES conjugate contains a malonate linker (Johnson et al., Anticancer Res. 15:1387-93 (1995)), a maleimidobenzoyl linker (Lau et al., Bioorg-Med-Chem. 3(10):1299-1304 (1995)), or a 3′-N-amide analog (Lau etal., Bioorg-Med-Chem. 3(10):1305-1312 (1995)).

In some embodiments, the T-Oligo-HES conjugate contains a linker that has an H-dimer forming fluorophore conjugated at the amino or carboxyl terminal residue. In some embodiments, the linker has an H-dimer forming fluorophore conjugated at the amino and carboxyl terminal residues. In some embodiments, the linker has a sulfhydryl or amino functional group at the amino and carboxyl terminus of the peptide;

In some embodiments, the T-Oligo-HES conjugate may be designed so that the Oligo-HES complex is delivered to the target but not released. This may be accomplished by attaching the Oligo-HES complex to the targeting moiety either directly or via a non-cleavable linker. In some embodiments, the T-Oligo-HES conjugate contains a linker that is non-cleavable. In some embodiments, the T-Oligo-HES conjugate contains an Oligo-HES complex that is directly linked to the targeting moiety.

These non-cleavable linkers may include amino acids, peptides, D-amino acids or other organic compounds that may be modified to include functional groups that can subsequently be utilized in attachment to ABs by the methods described herein.

Preferably, the linkers of the T-Oligo-HES conjugate are not substantially sensitive to the normal plasma environment. As used herein, “not substantially sensitive to the normal plasma environment,” in the context of a linker, means that no more than about 20%, typically no more than about 15%, more typically no more than about 10%, and even more typically no more than about 5%, no more than about 3%, or no more than about 1% of the linkers, in a sample of Targeted Oligo-HES conjugate compound, are cleaved when the Targeted Oligo-HES conjugate compound presents in the plasma of a normal subject. Whether a linker is not substantially sensitive to the normal plasma environment can be determined, for example, by incubating the Targeted Oligo-HES conjugate with plasma for a predetermined time period (e.g., 2, 4, 8, 16, or 24 hours) and then quantitating the amount of free Oligo-HES complex in the plasma.

In some embodiments, higher specific activity (or higher ratio of agents to AB) can be achieved by attachment of a single site linker at a plurality of sites on the AB. This plurality of sites may be introduced into the AB by either of two methods. First, one may generate multiple aldehyde groups and/or sulfhydryl groups in the same AB. Second, one may attach to an aldehyde or sulfhydryl of the AB a “branched linker” having multiple functional sites for subsequent attachment to linkers. The functional sites of the branched linker or multiple site linker may be aldehyde or sulfhydryl groups, or may be any chemical site to which linkers may be attached. Still higher specific activities may be obtained by combining these two approaches,that is, attaching multiple site. In addition, agents may be attached via disulfide bonds (for example, the disulfide bonds on a cysteine molecule) to the AB. Since many tumors naturally release high levels of glutathione (a reducing agent) this can reduce the disulfide bonds with subsequent release of the agent at the site of delivery. In some embodiments, the reducing agent that would modify a CM would also modify the linker of the conjugated activatable antibody.

### Methods of Manufacture

The disclosure utilizes several methods for conjugated Oligo-HES complexes to a targeting moiety including for example, (a) attachment to the carbohydrate moieties of the targeting moiety (e.g., antibody), (b) attachment to sulfhydryl groups of the targeting moiety (e.g., antibody), (c) attachment to amino groups of the targeting moiety (e.g., antibody), and (d) attachment to carboxylate groups of the targeting moiety (e.g., antibody). According to the disclosure, targeting moieties may be covalently attached to Oligo-HES complexes through an intermediate linker having at least two reactive groups, one to react with the targeting moiety and one to react with the Oligo-HES complexes. The linker, which may include any compatible organic compound, can be chosen such that the reaction with the targeting moiety and Oligo-HES complexes does not adversely affect the reactivity and selectivity of the targeting moiety or the activity of the Oligo-HES complexes.

Suitable linkers for reaction with oxidized polypeptide targeting moieties such as oxidized antibodies or oxidized antibody fragments include those containing an amine selected from the group consisting of primary amine, secondary amine, hydrazine, hydrazide, hydroxylamine, phenylhydrazine, semicarbazide and thiosemicarbazide groups. Such reactive functional groups may exist as part of the structure of the linker, or may be introduced by suitable chemical modification of linkers not containing such groups.

Suitable linkers for reaction with reduced polypeptide targeting moieties such as reduced antibodies or reduced antibody fragments include those having certain reactive groups capable of reaction with a sulfhydryl group of a reduced antibody or fragment. Such reactive groups include, but are not limited to: reactive haloalkyl groups (including, for example, haloacetyl groups), p- mercuribenzoate groups and groups capable of Michael -type addition reactions (including, for example, maleimides and groups of the type described by Mitra and Lawton, 1979, J. Amer. Chem. Soc. 101: 3097-3110).

Suitable linkers for attachment to neither oxidized nor reduced polypeptide such as antibodies or antibody fragments include those having certain functional groups capable of reaction with the primary amino groups present in unmodified lysine residues in the targeting moiety. Such reactive groups include, but are not limited to, NHS carboxylic or carbonic esters, sulfo- NHS carboxylic or carbonic esters, 4-nitrophenyl carboxylic or carbonic esters, pentafluorophenyl carboxylic or carbonic esters, acyl imidazoles, isocyanates, and isothiocyanates. In some embodiments, suitable linkers include those having certain functional groups capable of reacting with the carboxylic acid groups present in aspartate or glutamate residues in the targeting moiety, which have been activated with suitable reagents. Suitable activating reagents include EDC, with or without added NHS or sulfo-NHS, and other dehydrating agents utilized for carboxamide formation. In these instances, the functional groups present in the suitable linkers would include primary and secondary amines, hydrazines, hydroxylamines, and hydrazides.

The Oligo-HES complex may be attached to the linker before or after the linker is attached to the targeting moiety. In certain applications it may be desirable to first produce a targeting moiety-linker intermediate in which the linker is free of the Oligo-HES complex. Depending upon the particular application, the Oligo-HES complex agent may then be covalently attached to the linker.

### Methods of Treatment

The disclosure also provides T-Oligo-HES conjugates and methods for modulating nucleic acids and protein encoded or regulated by the oligonucleotides in the conjugates. In particular embodiments, the disclosure provides compositions and methods for modulating the levels, expression, processing or function of a mRNA, small non-coding RNA (e.g., miRNA), a gene, or a protein, in the targeted or localized cell(s) of a subject in vivo.

In some embodiments, the disclosure provides a method of targeting and delivering an oligonucleotide to a cell in vivo by administering to a subject in need thereof, a T-Oligo-HES conjugate containing the oligonucleotide. In particular embodiments, the oligonucleotide is a therapeutic oligonucleotide.

Thus, in some embodiments, the disclosure provides compositions, such as pharmaceutical compositions, comprising a T-Oligo-HES conjugate having (a) at least one oligonucleotide hybridizable with a target nucleic acid sequence under physiologic conditions, and (b) a targeting moiety that specifically binds a cell surface antigen on the cell or in the microenvironment of the cell containing the targeted nucleic acid.

In some embodiments, the disclosure provides a method for the targeted and/or localized delivery of an oligonucleotide to a subject in need thereof. In particular embodiments, the method comprises administering a T-Oligo-HES conjugate to a subject in need thereof, wherein the conjugate contains a therapeutically effective amount of an oligonucleotide sufficient to modulate a target RNA (e.g., mRNA and miRNA) or target gene, and a targeting moiety that specifically binds a cell surface antigen on the cell or in the microenvironment of the cell containing the target RNA or target gene.

According to one embodiment, the disclosure provides a method of modulating a target nucleic acid in a subject comprising administering a T-Oligo-HES conjugate to the subject, wherein an oligonucleotide of the complex comprises a sequence substantially complementary to the target nucleic acid that specifically hybridizes to and modulates levels of the nucleic acid or interferes with its processing or function, and the conjugate contains a targeting moiety that specifically binds a cell surface antigen on the cell or in the microenvironment of the cell containing the target nucleic acid. In some embodiments, the target nucleic acid is RNA, in further embodiments, the RNA is mRNA or miRNA. In further embodiments, the oligonucleotide reduces the level of a target RNA by at least 10%, at least 20%, at least 30%, at least 40% or at least 50% in one or more cells or tissues of the subject. In some embodiments, the target nucleic acid is a DNA.

According to one embodiment, the disclosure provides a method of modulating a protein in a subject comprising administering a T-Oligo-HES conjugate to the subject, wherein an oligonucleotide of the complex comprises a sequence substantially complementary to a nucleic acid that encodes the protein or influences the transcription, translation, production, processing or function of the protein, and a targeting moiety that specifically binds a cell surface antigen on the cell or in the microenvironment of the cell in which the protein is modulated. In some embodiments, the oligonucleotide specifically hybridizes to an RNA. In further embodiments, the RNA is mRNA or miRNA. In additional embodiments, the oligonucleotide reduces the level of the protein or RNA by at least 10%, at least 20%, at least 30%, at least 40% or at least 50% in one or more cells or tissues of the subject. In some embodiments, the oligonucleotide specifically hybridizes to a DNA.

In particular embodiments, the oligonucleotide in the T-Oligo-HES conjugate is selected from a siRNA, a shRNA, a miRNA, an anti-miRNA, a dicer substrate (e.g., dsRNA), an aptamer, a decoy, an antisense oligonucleotide, and a plasmid capable of expressing a siRNA, a miRNA, or an antisense oligonucleotide. In some embodiments, the oligonucleotide specifically hybridizes with an RNA or a sequence encoding an RNA. In other embodiments, the oligonucleotide specifically hybridizes with DNA sequence encoding an RNA or the regulatory sequences thereof.

In additional embodiments, the expression of a nucleic acid or protein is modulated in a subject by administering to a subject in need thereof, a T-Oligo-HES conjugate containing an antisense oligonucleotide, and a targeting moiety that specifically binds a cell surface antigen on the cell or in the microenvironment of the cell in which the nucleic acid or protein is modulated. In particular embodiments, the antisense oligonucleotide in the T-Oligo-HES conjugate is a substrate for RNAse H when bound to a target RNA. In some embodiments, the antisense oligonucleotide is a gapmer. As used herein, a “gapmer” refers an antisense compound having a central region (also referred to as a “gap” or “gap segment”) positioned between two external flanking regions (also referred to as “wings” or “wing segments”). The regions are differentiated by the types of sugar moieties comprising each distinct region. The types of sugar moieties that are used to differentiate the regions of a gapmer may in some embodiments, include beta-D-ribonucleosides, beta-D-deoxyribonucleosides, 2′-modified nucleosides (such 2′-modified nucleosides may include 2′-MOE, 2′-fluoro and 2′-O--CH3, among others), and bicyclic sugar modified nucleosides (such bicyclic sugar modified nucleosides may include LNA™ or ENA™, among others).

In some embodiments, each wing of a gapmer oligonucleotides comprises the same number of subunits. In other embodiments, one wing of a gapmer oligonucleotide comprises a different number of subunits than the other wing of the gapmer. In one embodiment, the wings of gapmer oligonucleotides have, independently, from 1 to about 5 nucleosides of which, 1, 2 3 4 or 5 of the wing nucleosides are sugar modified nucleosides. In one embodiment, the central or gap region contains 8-25 beta-D-ribonucleosides or beta-D-deoxyribonucleosides (i.e., is 8, 9, 10, 11, 12, 13, 14, 15, 16, 17, 18, 19, 20, 21, 22, 24 or 25 nucleosides in length). In a further embodiment, the central or gap region contains 17-24 nucleotides (i.e., is 17, 18, 19, 20, 21, 22, 23 or 24 nucleosides in length). In some embodiments, the gapmer oligonucleotide comprises phosphodiester internucleotide linkages, phosphorothioate internucleotide linkages, or a combination of phosphodiester and phosphorothioate internucleotide linkages. In particular embodiments, the central region of the gapmer oligonucleotide contains at least 2, 3, 4, 5 or 10 modified nucleosides, modified internucleoside linkages or combinations thereof. In particular embodiments, the central region of the gapmer oligonucleotide contains at least 10 beta-D-2′-deoxy-2′-fluororibofuranosyl nucleosides. In some embodiments, each nucleoside in the central region of the oligonucleotide a beta-D-2′-deoxy-2′-fluororibofuranosyl nucleoside. In one embodiment, the gapmer oligonucleotides is fully complementary over the length complementarity with the target RNA. In one embodiment, one or both wings of the gapmer contains at least one 2′ modified nucleoside. In one embodiment, one or both wings of the gapmer contains 1, 2 or 3 2′-MOE modified nucleosides. In one embodiment, one or both wings of the gapmer contains 1, 2 or 3 2′-OCH3 modified nucleosides. In another embodiment, one or both wings of the gapmer contains 1, 2 or 3 LNA or alpha-LNA nucleosides. In some embodiments, the LNA or alpha LNA in the wings of the gapmer contain one or more methyl groups in the (R) or (S) configuration at the 6′ (2′,4′-constrained-2′- O-ethyl BNA, S-cEt) or the 5′-position (-5′-Me-LNA or -5′-Me-alpha LNA) of LNA or alternatively contain a substituted carbon atom in place of the 2′-oxygen atom in the LNA or alpha LNA. In further embodiments, the LNA or alpha LNA in the gapmer contain a steric bulk moiety at the 5′ position (e.g., a methyl group). In a further embodiment, the gap comprises at least one 2′ fluoro modified nucleosides. In an additional embodiment, the wings are each 2 or 3 nucleosides in length and the gap region is 19 nucleotides in length. In additional embodiments, the gapmer has at least one 5-methylcytosine.

In another embodiment, the nucleosides of the central region (gap) contain uniform sugar moieties that are different than the sugar moieties in one or both of the external wing regions. In one non-limiting example, the gap is uniformly comprised of a first 2′-modified nucleoside and each of the wings is uniformly comprised of a second 2′-modified nucleoside. For example, in one embodiment, the central region contains 2′-F modified nucleotides flanked on each end by external regions each having two 2′-MOE modified nucleotides (2′-MOE/2′-F/2′-MOE). In particular embodiments, the gapmer is ISIS 393206. In another embodiment, the central region contains 2′-F modified nucleotides flanked on each end by external regions each having two 2′-MOE modified nucleotides (2′-MOE/2′-F/2′-MOE). In particular embodiments, the external regions each having two LNA or alpha LNA modified nucleotides in the wings of the gapmer. In further embodiments, the LNA or alpha LNA modified nucleotides contain one or more methyl groups in the (R) or (S) configuration at the 6′ (2′,4′-constrained-2′- O- ethyl BNA, S-cEt) or the 5′-position (-5′-Me-LNA or -5′-Me-alpha LNA) of LNA or alternatively contain a substituted carbon atom in place of the 2′-oxygen atom in the LNA or alpha LNA.

In another embodiment, the disclosure provides for the use of a T-Oligo-HES conjugates in the manufacture of a composition for the treatment of a disease or disorder. In another embodiment, the disclosure provides for the use of a T-Oligo-HES conjugates in the manufacture of a composition for the treatment of one or more of the conditions associated with a miRNA or a miRNA family.

According to one embodiment, the methods comprise the step of administering to or contacting the subject with a therapeutically effective amount of a T-Oligo-HES conjugate provided herein sufficient to modulate the target gene or RNA (e.g., mRNA and miRNA) expression and to thereby treat one or more conditions or symptoms associated with the disease or disorder. Exemplary compounds provided herein effectively modulate the expression, activity or function of the gene, mRNA or small-non-coding RNA target. In preferred embodiments, the small non-coding RNA target is a miRNA, a pre-miRNA, or a polycistronic or monocistronic pri-miRNA. In additional embodiments, the small non-coding RNA target is a single member of a miRNA family. In a further embodiment, two or more members of a miRNA family are selected for modulation.

In an additional embodiment, the disclosure provides a method of inhibiting the activity of a target nucleic acid in a subject, comprising administering to the subject a T-Oligo-HES conjugate comprising an oligonucleotide which is targeted to nucleic acids comprising or encoding the nucleic acid and which acts to reduce the levels of the nucleic acid and/or interfere with its function in the cell, and the conjugate further comprises a targeting moiety that specifically binds a cell surface antigen on the cell or in the microenvironment of the cell in which activity of the targeted nucleic acid is inhibited.

In particular embodiments, the target nucleic acid is a small-non coding RNA, such as, a miRNA. In some embodiments, the oligonucleotide comprises a sequence substantially complementary to the target nucleic acid.

In some embodiments, some embodiments, the disclosure provides a method of reducing expression of a target RNA in an subject in need of reducing expression of said target RNA, comprising administering to said subject an antisense T-Oligo-HES conjugate. In particular embodiments, an oligonucleotide in the complex is a substrate for RNAse H when bound to said target mRNA. In some embodiments, the oligonucleotide is a gapmer. As disclosed herein, oligonucleotides in the T-Oligo-HES conjugates provided herein display increased serum half-life. In particular embodiments, the serum half-life of an oligonucleotide in a T-Oligo-HES conjugate provided herein is greater than 10 minutes. In additional embodiments, the serum half-life of an oligonucleotide in a T-Oligo-HES conjugate provided herein is greater than 20, 30, 40, 50, 60, 90, 120, 180 or 200 minutes. In additional embodiments, the serum half-life of an oligonucleotide in a T-Oligo-HES conjugate provided herein is 30 to 300 minutes, 30 to 200 minutes or 30 to 120 minutes. In particular embodiments, the serum half-life of an oligonucleotide in a T-Oligo-HES conjugate provided herein is 1.5 to 4 times, 2 to 4 times, or 3 to 4 times that of the naked oligonucleotide (i.e., the oligonucleotide component not containing HES) in the serum alone. In other embodiments, the serum half-life of an oligonucleotide in a T-Oligo-HES conjugate provided herein is at least 1, 2, 3, or 4 hours longer than the serum half-life of the naked oligonucleotide in the serum alone. Techniques and methods for determining serum half-life are generally known in the art.

In an additional embodiment, the disclosure provides a method of reducing expression of a target RNA in a subject in need thereof, comprising administering to said subject a T-Oligo-HES conjugate containing an antisense oligonucleotide to said subject wherein the antisense sequence specifically hybridizes to the target RNA. In particular embodiments, the T-Oligo-HES conjugate comprises an antisense oligonucleotide that is a substrate for RNAse H when bound to a target RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′—O—(2-methoxyethyl)ribonucleotides.

In another embodiment, the antisense oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, each nucleoside of the oligonucleotide comprises a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide comprises at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (e.g., PMO). In some embodiments, the oligonucleotide sequence is specifically hybridizable to a sequence within 30 nucleotides of the AUG start codon of the target RNA. In additional embodiments, the oligonucleotide in the Oligo-HES complex sequence is specifically hybridizable to a sequence in the 5′ untranslated region of the target RNA. In some embodiments, the oligonucleotide in the Oligo-HES complex is designed to target the 3′ untranslated sequence in an RNA (e.g., mRNA). In further embodiments, the oligonucleotides in the oligonucleotide-HES complexes are designed to target the 3′ untranslated sequence in an RNA that is bound by a miRNA (i.e., the miRNA 3′UTR target site in an mRNA). One such example is “miR-Mask” or “target protector,” which are single-stranded 2′-O-methyl-modified (or other chemically modified) antisense oligonucleotide fully complementary to predicted miRNA binding sites in the 3′-UTR of a specific target mRNA, covering up the access of the miRNA to its binding site on the target mRNA (see, e.g., Choi et al., Science 318:271 (2007)); Wang, Methods Mol. Biol. 676:43 (2011)). In further embodiments, the oligonucleotides in the Oligonucleotide-HES complexes are designed to mimic the 3′ untranslated sequence in an mRNA that is bound by a miRNA. One such example is “miRNA sponges,” competitive miRNA inhibitory transgene expressing multiple tandem binding sites for an endogenous miRNA, which stably interact with the corresponding miRNA and prevent the association of target miRNA with its endogenous target mRNAs. In additional embodiments, the nucleic acid is an mRNA, and the oligonucleotide sequence is specifically hybridizable to a target region of a RNA selected from the group consisting of: an intron/exon junction of a target RNA, an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In another embodiment, the disclosure provides a method of inhibiting the production of a protein, comprising administering to a subject a T-Oligo-HES conjugate containing an oligonucleotide which is targeted to nucleic acids encoding the protein or decreases the endogenous expression, processing or function of the protein in the subject. In further embodiments, the administered conjugate comprises a targeting moiety that specifically binds a cell surface antigen on the cell or in the microenvironment of the cell in which the protein production is inhibited. In some embodiments, the oligonucleotide comprises a sequence substantially complementary to a nucleic acid encoding the protein.

In some embodiments, the disclosure provides a method of decreasing the amount of a target cellular RNA or corresponding protein in a cell by contacting a cell expressing the target RNA with a T-Oligo-HES conjugate having an oligonucleotide sequence that specifically hybridizes to the target RNA, wherein the amount of the target RNA or corresponding protein is reduced. In further embodiments, the administered conjugate comprises a targeting moiety that specifically binds a cell surface antigen on the cell or in the microenvironment of the cell in which the target cellular RNA or protein is decreased. In some embodiments, the RNA is an mRNA or a miRNA. In additional embodiments, the oligonucleotide is selected from a siRNA, a shRNA, a miRNA, an anti-miRNA, a dicer substrate (e.g., dsRNA), a decoy, an aptamer, a decoy, an antisense oligonucleotide and a plasmid capable of expressing a siRNA, a miRNA, an anti-miRNA, a ribozyme or an antisense oligonucleotide.

In particular embodiments, the oligonucleotide in the Oligonucleotide-HES complex is an antisense oligonucleotide. In one embodiment, the antisense oligonucleotide is a substrate for RNAse H when bound to a target RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides.

In another embodiment, the oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, each nucleoside of the oligonucleotide comprises a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide comprises at least one morpholino motif. In a further embodiment, the oligonucleotide comprises at least one phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In some embodiments, the oligonucleotides in the Oligo-HES complexes are designed to target the 3′ untranslated sequence in an RNA (e.g., mRNA). In further embodiments, the oligonucleotides in the Oligo-HES complexes are designed to target the 3′ untranslated sequence in an RNA that is bound by a miRNA. In additional embodiments, the target RNA is mRNA and the oligonucleotide sequence specifically hybridizes to a target region of the mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In some embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contain one or more modified nucleosides, modified internucleoside linkages, or combinations thereof.

In additional embodiments, the disclosure provides a method of reducing the expression of a target RNA in a subject in need thereof, comprising administering to the subject a T-Oligo-HES conjugate having an oligonucleotide sequence that specifically hybridizes to the target RNA, wherein the expression of the target RNA in a cell or tissue of the subject is reduced. In further embodiments, the administered conjugate comprises a targeting moiety that specifically binds a cell surface antigen on the cell or in the microenvironment of the cell in which the target RNA expression is reduced. In some embodiments, the RNA is an mRNA or a miRNA. In additional embodiments, the oligonucleotide is selected from a siRNA, shRNA, miRNA, an anti-miRNA, a dicer substrate, an aptamer, a decoy, an antisense oligonucleotide, a plasmid capable of expressing a siRNA, a miRNA, a ribozyme and an antisense oligonucleotide.

In particular embodiments, the oligonucleotide in the Oligo-HES complex is an antisense oligonucleotide. In one embodiment, the antisense oligonucleotide is a substrate for RNAse H when bound to the target RNA (e.g., mRNA and miRNA). In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides.

In another embodiment, the antisense oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, each of the nucleosides of the oligonucleotide comprise a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide contains at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence specifically hybridizes to a sequence in the 5′ untranslated region of the target RNA. In some embodiments, the oligonucleotide in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the target RNA is mRNA and the oligonucleotide sequence specifically hybridizes to a target region of the target mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In some embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

In some embodiments, a T-Oligo-HES conjugate is administered to a subject to provide a targeted and/or localized delivery an oligonucleotide that specifically hybridizes to a target nucleic acid (e.g., gene, mRNA or miRNA), which provides a growth advantage for a tumor cell or enhances the replication of a microorganism. In other embodiments, the T-Oligo-HES conjugate is administered to provide the targeted and/or localized delivery of an antisense, siRNA, shRNA, Dicer substrate or miRNA targeting an mRNA sequence coding for a protein (e.g., a protein variant) which has been implicated in a disease. Thus, in some embodiments, the disclosure provides a targeted and/or localized in vivo delivery system for delivering specific nucleic acid sequences into live cells to for example, silence genes in organisms afflicted with pathologic conditions due to aberrant gene expression.

In some embodiments, the disclosure provides a method of decreasing the amount of a polypeptide of interest in a cell, comprising: contacting a cell expressing a nucleic acid that encodes the polypeptide, or a complement thereof, with a T-Oligo-HES conjugate having an oligonucleotide sequence specifically hybridizes to a DNA or mRNA encoding the polypeptide, such that the expression of the polypeptide of interest is reduced. In further embodiments, the administered conjugate comprises a targeting moiety that specifically binds a cell surface antigen on the cell or in the microenvironment of the cell in which the amount of polypeptide is decreased. In further embodiments, the oligonucleotide is selected from a siRNA, shRNA, miRNA, an anti-miRNA, a dicer substrate, an antisense oligonucleotide, a plasmid capable of expressing a siRNA, a miRNA, a ribozyme and an antisense oligonucleotide, and wherein the oligonucleotide specifically hybridizes to a nucleic acid that encodes the polypeptide, or a complement thereof, such that the expression of the polypeptide is reduced. In particular embodiments, the oligonucleotide contains 12 to 30 linked nucleosides. In some embodiments, the complex contains a double-stranded RNA (dsRNA). In some embodiments, the oligonucleotide comprises at least one modified oligonucleotide. In further embodiments, the oligonucleotide comprises at least one modified oligonucleotide motif selected from a 2′ modification (e.g., 2′-fluoro, 2′-OME and 2′-methoxyethyl (2′-MOE)) a locked nucleic acid (LNA and alpha LNA), a PNA motif, and morpholino motif.

In particular embodiments, the oligonucleotide in the T-Oligo-HES conjugate is antisense sequence and is a substrate for RNAse H when bound to a target RNA. In additional embodiments, the antisense oligonucleotide is a gapmer. In some embodiments, the gapmer is an antisense oligonucleotide that is a chimeric oligonucleotide. In some embodiments, the chimeric oligonucleotide comprises a 2′-deoxynucleotide central gap region positioned between 5′ and 3′ wing segments. The wing segments contain nucleosides containing at least one 2′- modified sugar. The wing segments have nucleosides containing at least one 2′ sugar moiety selected from a 2′-O-methoxyethyl sugar moiety or a bicyclic nucleic acid sugar moiety. In some embodiments, the gap segment may be ten 2′-deoxynucleotides in length and each of the wing segments may be five 2′-O-methoxyethyl nucleotides in length. The chimeric oligonucleotide may be uniformly comprised of phosphorothioate internucleoside linkages. Further, each cytosine of the chimeric oligonucleotide may be a 5′-methylcytosine.

In another embodiment, the antisense oligonucleotide is not a substrate for RNAse H when hybridized to the RNA. In some embodiments, each nucleoside of the oligonucleotide comprises a modified sugar moiety comprising a modification at the 2′-position. In some embodiments, the oligonucleotide contains at least one PNA motif. In further embodiments, all the monomeric units of the oligonucleotide correspond to a PNA. In other embodiments, the oligonucleotide contains at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence specifically hybridizes to a sequence in the 5′ untranslated region of the target RNA. In some embodiments, the oligonucleotide in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the oligonucleotide sequence specifically hybridizes to a target region of a target mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In further embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

In an additional embodiment, the disclosure provides a method of increasing the activity of a nucleic acid in a subject, comprising administering to the subject a T-Oligo-HES conjugate containing an oligonucleotide which comprises or encodes the nucleic acid or increases the endogenous expression, processing or function of the nucleic acid (e.g., by binding regulatory sequences in the gene encoding the nucleic acid) and which acts to increase the level of the nucleic acid and/or increase its function in the cell. In further embodiments, the administered conjugate comprises a targeting moiety that specifically binds a cell surface antigen on the cell or in the microenvironment of the cell in which the activity of the nucleic acid is increased. In some embodiments, the oligonucleotide comprises a sequence substantially the same as nucleic acids comprising or encoding the nucleic acid.

In another embodiment, the disclosure provides a method of increasing the production of a protein, comprising administering to a subject a T-Oligo-HES conjugate containing an oligonucleotide which encodes the protein or increases the endogenous expression, processing or function of the protein in the subject. In some embodiments, the oligonucleotide comprises a sequence substantially the same as nucleic acids encoding the protein. In some embodiments, the oligonucleotide shares 100% identity with at least 15 contiguous nucleotides, at least 20 contiguous nucleotides or over the full-length of an endogenous nucleic acid sequence encoding the protein.

In some embodiments, the disclosure provides a method of treating a disease or disorder characterized by the overexpression of a nucleic acid in a subject, comprising administering to the subject a T-Oligo-HES conjugate containing an oligonucleotide which is targeted to a nucleic acid comprising or encoding the nucleic acid and which acts to reduce the levels of the nucleic acid and/or interfere with its function in the subject. In further embodiments, the administered conjugate comprises a targeting moiety that specifically binds a cell surface antigen on the cell or in the microenvironment of the cell in which the nucleic acid is overexpressed. In some embodiments, the nucleic acid is DNA, mRNA or miRNA, and the conjugate comprises a targeting moiety that specifically binds a cell surface antigen on the cell or in the microenvironment of the cell in which the protein production is inhibited. In additional embodiments, the oligonucleotide is selected from a siRNA, a shRNA, a miRNA, an anti-miRNA, a dicer substrate, an antisense oligonucleotide, a plasmid capable of expressing a siRNA, a miRNA, a ribozyme and an antisense oligonucleotide.

In particular embodiments, the nucleic acid is RNA and the oligonucleotide in the Oligo-HES complex is an antisense oligonucleotide. In one embodiment, the antisense oligonucleotide is a substrate for RNAse H when hybridized to the RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides. In some embodiments, the oligonucleotide comprises a sequence substantially complementary to the nucleic acid.

In another embodiment, the oligonucleotide is not a substrate for RNAse H when bound to the nucleic acid. In some embodiments, each nucleoside of the oligonucleotide comprises a modified sugar moiety comprising a modification at the 2′-position. In some embodiments, the oligonucleotide contains at least one PNA motif. In further embodiments, all the monomeric units of the oligonucleotide correspond to a PNA. In other embodiments, the oligonucleotide contains at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence specifically hybridizes to a sequence in the 5′ untranslated region of the target RNA. In some embodiments, the oligonucleotides in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the nucleic acid is mRNA and the oligonucleotide sequence specifically hybridizes to a target region of the mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In further embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

In further embodiments, the disclosure provides a method of treating a disease or disorder characterized by the overexpression of a protein in a subject, comprising administering to the subject a T-Oligo-HES conjugate containing an oligonucleotide that is targeted to a nucleic acid encoding the protein or decreases the endogenous expression, processing or function of the protein in the subject and the conjugate further comprises a targeting moiety that specifically binds a cell surface antigen on the cell or in the microenvironment of the cell in which the protein is overexpressed. In some embodiments, the nucleic acid is DNA, mRNA or miRNA. In additional embodiments, the oligonucleotide is selected from a siRNA, a shRNA, miRNA, an anti-miRNA, a dicer substrate, an aptamer, a decoy, an antisense oligonucleotide, a plasmid capable of expressing a siRNA, a miRNA, a ribozyme and an antisense oligonucleotide. In some embodiments, the oligonucleotide shares 100% identity with at least 15 contiguous nucleotides, at least 20 contiguous nucleotides or over the full-length of an endogenous nucleic acid sequence encoding the protein.

In particular embodiments, the targeted nucleic acid is RNA and the oligonucleotide in the Oligo-HES complex is an antisense oligonucleotide. In one embodiment, the antisense oligonucleotide is a substrate for RNAse H when hybridized to the RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides. In some embodiments, the oligonucleotide comprises a sequence substantially complementary to the nucleic acid.

In another embodiment, the oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, each nucleoside of the oligonucleotide comprises a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide comprises at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence is specifically hybridizable to a sequence in the 5′ untranslated region of the target RNA. (e.g., within 30 nucleotides of the AUG start codon) and to reduce translation. In some embodiments, the oligonucleotides in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the nucleic acid is mRNA and the oligonucleotide sequence specifically hybridizes to a target region of an mRNA encoding the protein selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In further embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In further embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

In some embodiments, the disclosure provides a method of treating (e.g., alleviating) a disease or disorder characterized by the aberrant expression of a protein in a subject, comprising administering to the subject a T-Oligo-HES conjugate containing an oligonucleotide which specifically hybridizes to the mRNA encoding the protein and alter the splicing of the target RNA (e.g., promoting exon skipping). In further embodiments, the administered conjugate comprises a targeting moiety that specifically binds a cell surface antigen on the cell or in the microenvironment of the cell in which the expression of the protein is aberrant. In some embodiments, each nucleoside of the oligonucleotide comprises at least one modified sugar moiety comprising a modification at the 2′-position. In particular embodiments, the modified oligonucleotide is a 2′ OME or 2′ allyl. In additional embodiments, the modified oligonucleotide is LNA, alpha LNA (e.g., an LNA or alpha LNA containing a steric bulk moiety at the 5′ position (e.g., a methyl group). In some embodiments, the oligonucleotide contains at least one PNA motif. In further embodiments, all the monomeric units of the oligonucleotide correspond to a PNA. In other embodiments, the oligonucleotide contains at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence specifically hybridizes to a sequence in the 5′ untranslated region of the target RNA. In some embodiments, the oligonucleotides in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, oligonucleotide sequence is specifically hybridizable to a target region of an mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction.

In particular embodiments, the disease or disorder is Duchenne Muscular Dystrophy (DMD). In some embodiments, the oligonucleotide specifically hybridizes to mRNA sequence that promotes message splicing to “skip over” exon 44, 45, 50, 51, 52, 53 or 55 of the dystrophin gene. In particular embodiments, the oligonucleotide specifically hybridizes to mRNA sequence that promotes message splicing to “skip over” exon 51 of the dystrophin gene. In particular embodiments, the oligonucleotide in the T-Oligo-HES conjugate is AVI-4658 (AVI Biopharma). In other embodiments, the oligonucleotide in the T-Oligo-HES conjugate competes for dystrophin mRNA binding with AVI-4658. In particular embodiments, the oligonucleotide in the T-Oligo-HES conjugate is eteplirsen or drisapersen. In other embodiments, the oligonucleotide in the T-Oligo-HES conjugate competes for dystrophin mRNA binding with eteplirsen or drisapersen.

A further embodiment, of the disclosure provides a method comprising, selecting a subject who has received a diagnosis of a disease or disorder, administering to the subject a therapeutically effective amount of a T-Oligo-HES conjugate containing an oligonucleotide that specifically hybridizes to a nucleic acid sequence believed to be associated with or to encode a protein associated with the disease or disorder or a condition related thereto, and monitoring disease progression in the subject. In further embodiments, the administered conjugate comprises a targeting moiety that specifically binds a cell surface antigen on the cell or in the microenvironment of the cell containing the nucleic acid.

In some embodiments, the nucleic acid is DNA, mRNA or miRNA. In additional embodiments, the oligonucleotide is selected from a siRNA, a shRNA, a miRNA, an anti-miRNA, a dicer substrate, an aptamer, a decoy, and an antisense oligonucleotide, a plasmid capable of expressing a siRNA, a miRNA, a ribozyme and an antisense oligonucleotide. In some embodiments, the oligonucleotide shares 100% identity with at least 15 contiguous nucleotides, at least 20 contiguous nucleotides or over the full-length of the nucleic acid.

In particular embodiments, the nucleic acid is RNA and the oligonucleotide in the Oligo-HES complex is an antisense oligonucleotide. In one embodiment, the antisense oligonucleotide is a substrate for RNAse H when hybridized to the RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides. In some embodiments, the oligonucleotide comprises a sequence substantially complementary to the nucleic acid.

In another embodiment, the oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, all the nucleosides of the oligonucleotide comprise a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide comprises at least one morpholino motif. In some embodiments, the morpholino is a phosphorodiamidate morpholino. In additional embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence specifically hybridizes to a sequence in the 5′ untranslated region of the target RNA. In some embodiments, the oligonucleotides in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the oligonucleotide specifically hybridizes to a target region of the mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction. In additional embodiments, the T-Oligo-HES conjugate contains an oligonucleotide that specifically hybridizes to nucleotides 1-10 of a miRNA (i.e., the seed region) or that specifically hybridizes to a sequence in a precursor-miRNA (pre-miRNA) or primary-miRNA (pri-miRNA) that when bound by the oligonucleotide blocks miRNA processing.

In further embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

In another embodiment, the disclosure provides a method of slowing disease progression in a subject suffering from a disease or disorder correlated with the overexpression of a protein comprising, administering to the subject a T-Oligo-HES conjugate containing an oligonucleotide that specifically hybridizes to a DNA or mRNA encoding the protein, such that the expression of the polypeptide is reduced. In further embodiments, the administered conjugate comprises a targeting moiety that specifically binds a cell surface antigen on the cell or in the microenvironment of the cell in which the protein is overexpressed. In additional embodiments, the oligonucleotide is selected from a siRNA, a shRNA, a miRNA, an anti-miRNA, a dicer substrate, an antisense oligonucleotide, a plasmid capable of expressing a siRNA, a miRNA, a ribozyme and an antisense oligonucleotide. In some embodiments, the oligonucleotide shares 100% identity with at least 15 contiguous nucleotides, at least 20 contiguous nucleotides or over the full-length of the DNA or mRNA encoding the protein.

In particular embodiments, the nucleic acid is mRNA and the oligonucleotide in the Oligo-HES complex is an antisense oligonucleotide. In one embodiment, the antisense oligonucleotide is a substrate for RNAse H when hybridized to the RNA. In additional embodiments, the antisense oligonucleotide is a gapmer. In some embodiments, the oligonucleotide is 18 to 24 nucleotides in length comprising: a gap region having greater than 11 contiguous 2′-deoxyribonucleotides; and a first wing region and a second wing region flanking the gap region, wherein each of said first and second wing regions independently have 1 to 8 2′-O-(2-methoxyethyl)ribonucleotides. In particular embodiments, the oligonucleotide contains 12 to 30 linked nucleosides. In some embodiments, the oligonucleotide comprises a sequence substantially complementary to the nucleic acid.

In another embodiment, the oligonucleotide is not a substrate for RNAse H when bound to the target RNA (e.g., mRNA and miRNA). In some embodiments, the oligonucleotide comprises at least one modified sugar moiety comprising a modification at the 2′-position. In some embodiments, each nucleoside of the oligonucleotide comprises a modified sugar moiety comprising a modification at the 2′-position. In some embodiments, the oligonucleotide comprises at least one PNA motif. In further embodiments, all the monomeric units of the oligonucleotide correspond to a PNA. In other embodiments, the oligonucleotide comprises at least one morpholino motif. In some embodiments, the morpholino is a phosphorodiamidate morpholino. In further embodiments, all the monomeric units of the oligonucleotide correspond to a morpholino. In further embodiments, all the monomeric units of the oligonucleotide correspond to a phosphorodiamidate morpholino (PMO). In some embodiments, the oligonucleotide sequence specifically hybridizes to a sequence within 30 nucleotides of the AUG start codon of the target RNA. In additional embodiments, the oligonucleotide sequence is specifically hybridizable to a sequence in the 5′ untranslated region of the target RNA. In some embodiments, the oligonucleotides in the Oligo-HES complexes are designed to target the 3′ untranslated sequence in an RNA (e.g., mRNA). In further embodiments, the oligonucleotides are designed to target the 3′ untranslated sequence in an RNA that is bound by a miRNA. In additional embodiments, the nucleic acid is an mRNA and the oligonucleotide sequence specifically hybridizes to a target region of the mRNA selected from the group consisting of: an intron/exon junction of a target RNA, and an intron/exon junction and a region 1 to 50 nucleobases 5′ of an intron/exon junction of the target RNA. In some embodiments, the target region is selected from the group consisting of: a region 1 to 15 nucleobases 5′ of an intron/exon junction, 20 to 24 nucleobases 5′ of an intron/exon junction, and 30 to 50 nucleobases 5′ of an intron/exon junction.

In further embodiments, the oligonucleotide can induce RNA interference (RNAi). In some embodiments, the oligonucleotide is siRNA, shRNA or a Dicer substrate. In some embodiments, the oligonucleotide is a siRNA that is 18-35 nucleotides in length. In some embodiments, the oligonucleotide is a shRNA that has a stem of 19 to 29 nucleotides in length and a loop size of between 4-30 nucleotides. In further embodiments, the siRNA or shRNA oligonucleotide contains one or more modified nucleosides, modified internucleoside linkages, or combinations thereof. In some embodiments, the oligonucleotide is a Dicer substrate and contains 2 nucleic acid strands that are each 18-25 nucleotides in length and contain a 2 nucleotide 3′ overhang. In particular embodiments, the Dicer substrate is a double stranded nucleic acid containing 21 nucleotides in length and contains a two nucleotide 3′ overhang. In further embodiments, one or both strands of the Dicer substrate contains one or more modified nucleosides, modified internucleoside linkages, or combinations thereof.

There currently exist several distinct groups of pathological conditions that are known to be regulated by a miRNA or a family of miRNA, which can be targeted using the T-Oligo-HES conjugates provided herein.

In one embodiment, the T-Oligo-HES conjugate contains an oligonucleotide that inhibits or mimics one or more miRNAs associated with an infectious disease. In one embodiment, the oligonucleotide inhibits miR-122. Miravirsen (SPC3649), an inhibitor of miR-122 developed by Santaris Pharma A/S. Mir-122 is a liver specific miRNA that the Hepatitis C virus requires for replication as a critical endogenous host factor. Clinical trial data for 4-week Miravirsen monotherapy has shown robust dose-dependent anti-viral activity. Regulus Therapeutics and GlaxoSmithKline (GSK) have likewise demonstrated in a preclinical study that miR-122 is essential in the replication of HCV and plan to advance an anti-miR-122 into clinical studies for the treatment of HCV infection.

In another embodiment, the T-Oligo-HES conjugate contains an oligonucleotide that inhibits or mimics a miRNA associated with fibrosis. In one embodiment, the inhibits miR-21. Preclinical studies by Regulus Pharmaceutical and Sanofi Aventis have shown that inhibition of miR-21, which is upregulated in human fibrotic tissues, can improve organ function in multiple models of fibrosis including heart and kidney. In another embodiment, the oligonucleotide corresponds to or mimics miR-29. MGN-4220, mimics or miRNA replacement therapy by Mirna Therapeutics, targets miR-29 implicated in cardiac fibrosis.

In another embodiment, the T-Oligo-HES conjugate contains an oligonucleotide that inhibits or mimics a miRNA associated with a cardiovascular disease, including, but not limited to, stroke, heart disease, atherosclerosis, restenosis, thrombosis, anemia, leucopenia, neutropenia, thrombocytopenia, granuloctopenia, pancytoia and idiopathic thrombocytopenic purpura. In one embodiment, an oligonucleotide inhibits miR-33. Regulus Pharmaceutical and AstraZeneca has shown in preclinical studies that the inhibition of miR-33 reduces arterial plaque size and increase levels of HDL. In another embodiment, the oligonucleotide inhibits miR-92, miR-378, miR-206 and/or the miR-143/145 family. MGN-6114, MGN-5804, MGN-2677, MGN-8107, developed by Miragen Therapeutics, respectively targets miR-92 implicated in peripheral arterial disease, miR-378 implicated in cardiometablolic disease, miR-143/145 family implicated in vascular disease, and miR-206 implicated in amylotrophic lateral sclerosis. In a further embodiment, the oligonucleotide inhibits the miR-208/209 family and/or the miR-15/195 family. Miragen Therapeutics’s MGN-9103 and MGN-1374 are miRNA inhibitors that respectively target miR-208/209 family for chronic heart failure and miR-15/195 family for post-myocardial infarction remodeling. In another embodiment, an oligonucleotide in the T-Oligo-HES conjugate inhibits miR-126 and/or miR92a. miR-126 and miR-92a play central roles in the development of an atherosclerotic plaque.

In another embodiment, the T-Oligo-HES conjugate contains an oligonucleotide that inhibits a miRNA associated with a neurological disease or condition. In one embodiment, the oligonucleotide inhibits miR-206. miR-206 plays a crucial role in ALS and in neuromuscular synapse regeneration.

In another embodiment, the T-Oligo-HES conjugate contains an oligonucleotide that inhibits or mimics a miRNAs associated with oncological conditions. In one embodiment, the oligonucleotide inhibits miR-21. miR-21 has been suggested by numerous scientific publications to play an important role in the initiation and progression of cancers including liver, kidney, breast, prostate, lung and brain. Anti-miR-21 in hepatocellular carcinoma (HCC) mouse model has shown delayed tumor progression in a preclinical study by Regulus Pharmaceutical and Sanofi Aventis. In another embodiment, the oligonucleotide inhibits miR-10b. Preclinical animal studies of anti-miR-10b by Regulus Pharmaceutical also showed therapeutic effect in GBM model. In another embodiment, the T-Oligo-HES conjugate contains an oligonucleotide that corresponds to or mimics miR-34. Mimics or miRNA replacement therapy by Mirna Therapeutics of miR-34, which is lost or expressed at reduced levels in most solid and hematologic malignancies, showed inhibition of growth for various types of cancers in preclinical studies of MRX34.

In some embodiments, the T-Oligo-HES conjugate contains an oligonucleotide that inhibits a miRNA selected from: let-7a, miR-9, miR-10b, miR-15a-miR-16-1, miR-16, miR-21, miR-24, miR-26a, miR-34a, miR-103-107, miR-122, miR-133, miR-181, miR-192, miR-194, miR-200. These microRNAs are among those that have been reported to be associated with cancer.

In some embodiments, the T-Oligo-HES conjugate contains an oligonucleotide that inhibits a miRNA selected from: let-7, let-7a, let-7f, miR-1, Mir-10b, miR-15a-miR-16-1, Mir-17-5p, Mir-17-92, miR-21, Mir-23-27, miR-25, miR-27b, miR-29, miR-30a, Mir-31, miR-34a, miR-92-1, miR-106a, miR-125, Mir-126, Mir-130a, Mir-132, miR-133b, Mir-155, miR-206, Mir-210, Mir-221/222, miR-223, Mir-296, miR-335, Mir-373, Mir-378, miR-380-5p, Mir-424, miR-451, miR-486-5p, and Mir-520c. These microRNAs are among those that have been reported to promote neovascularization, metastasis and/or the onset of cancer.

In some embodiments, the T-Oligo-HES conjugate contains an oligonucleotide that inhibits a miRNA selected from: miR-15 family, miR-21, miR-23, miR-24, miR-27, miR-29, miR-33, miR-92a, miR-145, miR-155, miR-199b, miR-208a/b family, miR-320, miR-328, miR-499. These microRNAs are among those that have been reported to have various roles in cardiovascular functions.

In some embodiments, the T-Oligo-HES conjugate contains an oligonucleotide that inhibits a miRNA selected from: let-7b, miR-9, miR106b-25 cluster, miR-124, miR-132, miR-137, miR-184. These microRNAs are among those that have been reported to have various roles in adult neurogenesis in neural stem cells (NSCs).

In some embodiments, the T-Oligo-HES conjugate contains an oligonucleotide that inhibits or mimics a miRNA selected from: let-7a, miR-21, mir-26, miR-125b, mir-145, miR-155, miR-191, miR-193a, miR-200 family, miR-205, miR-221, and miR-222. These microRNAs are among those that have been reported to function as diagnostic or prognostic biomarkers for various types of cancers. In particular embodiment, the oligonucleotide inhibits a miRNA selected from: miR-21, mir-26, miR-125b, miR-155, miR-193a, miR-200 family, miR-221, and miR-222. In particular embodiment, the oligonucleotide contains the sequence of, or mimics a miRNA selected from: let-7a, mir-145, miR-191, and miR-205.

In some embodiments, the T-Oligo-HES conjugate contains an oligonucleotide that inhibits a miRNA selected from: miR-138, mir-182, miR-21, mir-103/107, and miR-29c. These microRNAs are among those that have been reported to have roles in arthritis, lupus, atherosclerosis, insulin sensitivity, and albuminuria, respectively.

In some embodiments, the T-Oligo-HES conjugate contains an oligonucleotide that inhibits or mimics a miRNA selected from: let-7, let-7-a3, lin-28, miR-1, miR-9-1, miR-15a, miR-16-1, miR-17-92 cluster, miR-21, miR-29 family, miR-34 family, miR-124, miR-127, and miR-290. These microRNAs are among those that have been reported to be dysregulated in various types of cancers due to abnormalities in genetic or epigenetic regulations responsible for miRNA expression. In particular embodiment, the oligonucleotide inhibits a miRNA selected from: let-7-a3, lin-28, miR-17-92 cluster, and miR-21. In particular embodiment, the oligonucleotide contains the sequence of, or mimics a miRNA selected from: let-7, miR-1, miR-9-1, miR-15a, miR-16-1, miR-21, miR-29 family, miR-34 family, miR-124, miR-127, and miR-290. In a particular embodiment, the T-Oligo-HES conjugate contains an oligonucleotide that inhibits miR-138.

In some embodiments, the T-Oligo-HES conjugate contains an oligonucleotide that the sequence of, or mimics a miRNA selected from: Mir-20a, Mir-34, Mir-92a, Mir-200c, Mir-217 and Mir-503. These miRNAs are among those that have been reported to be antiangiogenic.

In some embodiments, the T-Oligo-HES conjugate contains an oligonucleotide that the sequence of, or mimics: miR-1, miR-2, miR-6, miR-7 or let-7. In particular embodiments, the oligonucleotides are miR-Rx07, miR-Rx06, miR-Rxlet-7, miR-Rx01, miR-Rx02 or miR-Rx03. In an additional embodiment, an oligonucleotide in the T-Oligo-HES conjugate corresponds to or mimics miR-451. miR-451 has been demonstrated to regulate erythropoiesis in vivo (Patrick et al., Genes & Dev., 24: 1614-1619 (2010)) and thus to be implicated in diseases such as, polycythemia vera, red cell dyscrasias generally, or other hematopoietic malignancies. In particular embodiments, the oligonucleotide is MGN-4893.

In additional embodiments, pharmaceutical compositions containing a T-Oligo-HES conjugate comprising an antisense oligonucleotide targeted to a nucleic acid of interest are used for the preparation of a composition for treating a patient suffering or susceptible to a disease or disorder associated with the nucleic acid.

### Exemplary Therapeutic Applications of T-Oligo-HES Conjugates

As will be immediately apparent to a person of skill in the art, due in part to the surprising highly efficient systemic in vivo delivery of oligonucleotides into cells, the T-Oligo-HES conjugates provided herein essentially have limitless applications in modulating target nucleic acid and protein levels and activity and are particularly useful in therapeutic applications.

Non limiting examples of diseases and disorders that can be treated with T-Oligo-HES conjugates include, a proliferative disorder (e.g., a cancer, such as hematological cancers (e.g., AML, CML, CLL and multiple myeloma) and solid tumors (e.g., melanoma, renal cancer, pancreatic cancer, prostate cancer, ovarian cancer, breast cancer, NSCLC,), immune (e.g., ulcerative colitis, Crohn’s disease, IBD, psoriasis, asthma, autoimmune diseases such as rheumatoid arthritis, multiple sclerosis, and SLE) and inflammatory diseases, neurologic diseases (e.g., diabetic retinopathy, Duchenne’s muscular dystrophy, myotinic dystrophy, Huntington’s disease and spinal muscular atrophy and other neurodegenerative diseases), metabolic diseases (e.g., type II diabetes, obesity), cardiovascular diseases (e.g., clotting disorders, thrombosis, coronary artery disease, restenosis, amyloidosis, hemophilia, anemia, hemoglobulinopathies, atherosclerosis, high cholesterol, high tryglycerides), endocrine related diseases and disorders (e.g., NASH, diabetes mellitus, diabetes insipidus, Addison’s disease, Turner syndrome, Cushing’s syndrome, osteoporosis) and infectious disease. Thus, in one embodiment, the disclosure provides a method of treating a disease in a subject comprising administering to a subject that has been diagnosed with the disease, a therapeutically effective amount of a T-Oligo-HES conjugate containing a therapeutic oligonucleotide that specifically hybridizes to a nucleic acid associated with the disease or disorder or a symptom thereof.

In additional embodiments, the disease or disorder treated with a T-Oligo-HES conjugate provided herein is a disease or disorder of the kidneys, liver, lymph nodes, spleen or adipose tissue.

The disclosure also provides a method of monitoring the delivery of a therapeutic oligonucleotide to a cell or tissue in a subject, comprising administering to the subject a T-Oligo-HES conjugate containing a therapeutic oligonucleotide and monitoring the fluorescence of cells or tissue in the subject, wherein an increased fluorescence in the cells or tissue of the subject indicates that the therapeutic oligonucleotide has been delivered to the cells or tissue of the subject.

In some embodiments, the disclosure provides a method of treating a disease or disorder characterized by the under expression of a nucleic acid in a subject, comprising administering to the subject a T-Oligo-HES conjugate containing an oligonucleotide which comprises or encodes the nucleic acid or increases the endogenous expression, processing or function of the nucleic acid (e.g., by binding regulatory sequences in the gene encoding the nucleic acid) and which acts to increase the level of the nucleic acid and/or increase its function in the cell. In some embodiments, the oligonucleotide comprises a sequence substantially the same as a nucleic acid comprising or encoding the nucleic acid.

In some embodiments, the disclosure provides a method of treating a disease or disorder characterized by the underexpression of a protein in a subject, comprising administering to the subject a T-Oligo-HES conjugate containing an oligonucleotide which encodes the protein or increases the endogenous expression, processing or function of the protein in the subject.

In another embodiment, the disclosure provides a method of treating cancer or one or more conditions associated with cancer by administering a therapeutically effective amount of a T-Oligo-HES conjugate to a subject in need thereof. “Cancer,” “tumor,” or “malignancy” are used herein as synonymous terms and refer to any of a number of diseases that are characterized by uncontrolled, abnormal proliferation of cells, the ability of affected cells to spread locally or through the bloodstream and lymphatic system to other parts of the body (metastasize), as well as any of a number of known characteristic structural and/or molecular features. A “cancerous tumor” or “malignant cell” is understood as a cell having specific structural properties, lacking differentiation and being capable of invasion and metastasis. Examples of cancers that may be treated using T-Oligo-HES conjugates provided herein include solid tumors and hematologic cancers. Additional, examples of cancers that can be treated using T-Oligo-HES conjugates provided herein include, breast, lung, brain, bone, liver, kidney, colon, head and neck, ovarian, hematopoietic (e.g., leukemia), and prostate cancer. Further examples of cancer that can be treated using T-Oligo-HES conjugates include, but are not limited to, carcinoma, lymphoma, myeloma, blastoma, sarcoma, and leukemia. More particular examples of such cancers include, but are not limited to, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. In a particular embodiment, the T-Oligo-HES conjugates are used to treat a leukemia. In another particular embodiment, the T-Oligo-HES conjugates are used to treat metastatic cancer.

In additional embodiments, a therapeutically effective amount of a T-Oligo-HES conjugate is administered to treat a hematologic cancer. In further embodiments, the, T-Oligo-HES conjugate is administered to treat a cancer selected from: lymphoma, leukemia, myeloma, lymphoid malignancy, cancer of the spleen, and cancer of the lymph nodes. In additional embodiments, a therapeutically effective amount of a T-Oligo-HES conjugate is administered to treat a lymphoma selected from: Burkitt’s lymphoma, diffuse large cell lymphoma, follicular lymphoma, Hodgkin’s lymphoma, mantle cell lymphoma, marginal zone lymphoma, mucosa-associated-lymphoid tissue B cell lymphoma, non-Hodgkin’s lymphoma, small lymphocytic lymphoma, and a T cell lymphoma. In additional embodiments, a therapeutically effective amount of a T-Oligo-HES conjugate is administered to treat a leukemia selected from: chronic lymphocytic leukemia, B cell leukemia (CD5+ B lymphocytes), chronic myeloid leukemia, lymphoid leukemia, acute lymphoblastic leukemia, myelodysplasia, myeloid leukemia, acute myeloid leukemia, and secondary leukemia. In additional embodiments, a therapeutically effective amount of a T-Oligo-HES conjugate is administered to treat multiple myeloma. Other types of cancer and tumors that can be treated using T-Oligo-HES conjugates are described herein or otherwise known in the art.

In particular embodiments, the T-Oligo-HES conjugate contains an oligonucleotide selected from: AVI-4557 (Cyp 3A4m; AVI Biopharma), ISIS-23722 (Survivin; ISIS); Gem-640 (XIAP; Hybridon), Atu027 (PKN3; Silence Therapeutics), CEQ508 (B catenin; Marina Biotech), GEM 231 (PKA R1α subunit; Idera), Affinitak (Aprinocarsen, ISIS 3521/LY900003; PKC-α; ISIS/Lilly); Aezea (OL(1)P53/EL-625; P53; Eleos Pharma); ISIS 2503 (H-ras; ISIS), EZN-2968 (HIF-1α; Enzon Pharmaceuticals); G4460/ LR 3001 (c-Myb; Inex/Genta); LErafAON (c-Raf; NeoPharm), ISIS 5132 (c-Raf; ISIS), Genasense (Oblimersen/G3139; Bcl2; Genta); SPC2996 (Bcl2; Santaris Pharma), OGX-427 (Hsp27; ISIS/ OncoGene X), LY2181308 (Surivin; Lilly), LY2275796 (EIF4E ; Lilly), ISIS-STAT3 Rx (STAT3; ISIS), OGX-011 (Custirsen; clusterin; Teva), Veglin (VEGF; VasGene Therapeutics, AP12009 (TGF-β2; Antisense Pharma), GTI-2501 (Ribonucleotide Reductase R1; Lorus Therapeutics), Gem-220 (VEGF; Hybridon); Gem-240 (MEM2; Hybridon), CALAA-19 (M2 subunit ribonucleotide reductase; Arrowhead Research Corporation), Trabedersen (AP 12009; TGF-β2; Antisense), GTI-2040 (Ribonucleotide Reductase R2, Lorus Therapeutics (5′-GGCTAAATCGCTCCACCAAG-3′) (SEQ ID NO:9)), AEG 35156 (XIAP; Aegera Pharma), and MG 98 (DNA methyltransferase; MethylGene/MGI Pharma/British Biotech). In particular embodiments, the oligonucleotide in conjugate competes for target nucleic acid binding with one of the above oligonucleotides.

In additional embodiments, the T-Oligo-HES conjugate contains an oligonucleotide selected from: Atu027 (PKN3), TKM-PLK1 (PLK1), ALN-VSP02 (KSP and VEGF), CALAA-01 (RRM2), siG12D LODER (K-ras), ISIS-EIF4ERx (EIFR), GTI-2040 (RRM2), Trabedersen (TGFB2), Archexin (Protein kinase B alpha (Akt1)), and Cenersen (P53); In particular embodiments, the oligonucleotide in conjugate competes for target nucleic acid binding with one of the above oligonucleotides.

In additional embodiments, the T-Oligo-HES conjugate contains an oligonucleotide having a sequence selected from: 5′-GTTCTCGCTGGTGAGTTTCA-3′ (SEQ ID NO:2) (PKC-α); 5′-CCCTGCTCCCCCCTGGCTCC-3′ (SEQ ID NO:3)(P53); 5′-TCCGTCATC GCTCCTCAGGG-3′ (SEQ ID NO:4)(H-ras); 5′-GGGACTCCTCGCTACTGCCT-3′ (SEQ ID NO:5)(H-ras); 5′-TCCCGCCTGTGACATGCATT-3′ (SEQ ID NO:6)(c-Raf); 5′-TCTCCCAGCGTGCGCCAT-3′ (SEQ ID NO:7)(Bcl2); and 5′-TGGCTTGAAGAT GTACTCGAT-3 (SEQ ID NO:8)(TGF-β2). In particular embodiments, the oligonucleotide in conjugate competes for target nucleic acid binding with one of the above oligonucleotides.

In additional embodiments, the T-Oligo-HES conjugate contains an oligonucleotide having a sequence selected from: 5′-TATGCTGTGCCGGGGTCTTCGGGC-3′ (SEQ ID NO:10)(c-myb); 5′-TCCCGCCTGTGACATGCATT-3′ (SEQ ID NO:6)(c-RAF); 5′-CGC TGAAGGGCTTCTTCCTTATTGAT-3′ (SEQ ID No:1 1)(Bcr-abl); 5′-CGCTGAAGGGCT TTGAACTGTGCTT-3′ (SEQ ID NO:12)(Bcr-abl); 5′-GGGACTCCTCGCTACTGCCT-3′ (SEQ ID NO:5) (Ha-Ras); 5′-GCGUGCCTCCTCACUGGC-3′ (SEQ ID NO:13) (Pka-rIA); 5′-AACGTTGAGGGGCAT-3′ (SEQ ID NO: 14)(c-Myc); 5′-GCTCAGTGGACATGGAT GAG-3′ (SEQ ID NO:15)(JNK2); 5′-GGACCCTCCTCCGGAGCC-3′ (SEQ ID NO:16)(IGF-1R); 5′-TGACTGTGAACGTTCGAGATGA-3′ (SEQ ID NO:18)(TLR-9); and 5′-CTGC TAGCCTCTGGATTTGA-3′ (SEQ ID NO:17)(PTEN). In particular embodiments, the oligonucleotide in conjugate competes for target nucleic acid binding with one of the above oligonucleotides.

In additional embodiments, the T-Oligo-HES conjugate contains an oligonucleotide having the sequence 5′- TAGGAAAAGCTATTAGGAGTCTTTATAGTATA - 3’ (SEQ ID NO:79) (K-ras). In further embodiments, the T-Oligo-HES conjugate contains an oligonucleotide having the sequence 5’- [5’ Disulfide C6 linker attached to 5’ OH of dT with phosphate] [C6 Amino dT] AGGAAAAG*C¹T*ATTAGGAGTCT*T*T*A TAGTAT²A - 3’ (SEQ ID NO:79), wherein * is an LNA, ¹ is LNA-5Me, and ² is C6amino dT . In further embodiments, the T-Oligo-HES conjugate contains phosphorothioate backbones in all linkages.

In some embodiments, the T-Oligo-HES conjugate contains an oligonucleotide having the sequence 5′- AUACUAAAUCAUUUGAAGAUAUUCACCAUT - 3’ (SEQ ID NO:84) (K-ras). In further embodiments, the T-Oligo-HES conjugate contains an oligonucleotide having the sequence 5′- A^{X}U^{X}A^{X}CUAAAUCAUUUGAAGAUAUUCACCA^{X}U^{X}-[C6Amino dT] - 3’ (SEQ ID NO:84)), wherein ^{x} denotes a 2′-MOE. In some embodiments, the T-Oligo-HES conjugate contains an oligonucleotide having the sequence 5′- TAUGGUGAAU AUCUUCAAAUGAUU UAGUAU - 3’ (SEQ ID NO:85) (K-ras). In further embodiments, the T-Oligo-HES conjugate contains an oligonucleotide having the sequence 5′- [C6Amino dT]A^{x}U^{x}GGUGAAUAUCU^{x}UC^{x}AAA^{x}U^{x}G^{x}A^{x}U^{x}U ^{x}U^{x}A^{x}G^{x}U^{x}A^{x}U^{x} - 3’ (SEQ ID NO:85), wherein ^{x} denotes a 2′-MOE. In some embodiments, the T-Oligo-HES conjugate contains a duplex oligonucleotide having the sequence 5′- AUACUAAAUCA UUUGAAGAUAUUC ACCAUT-3′ (SEQ ID NO:84) and an oligonucleotide having the sequence 5′-TAUGGUGAAUAUCUUCAAAUGAUU UAGUAU - 3′ (SEQ ID NO:85).

In some embodiments, the T-Oligo-HES conjugate contains an oligonucleotide having the sequence 5′-AGUACAGUGCAAUGAGGGACCAGUACAUGAT-3′ (SEQ ID NO:86) (K-ras). In further embodiments, the T-Oligo-HES conjugate contains an oligonucleotide having the sequence 5′-A^{x}G^{x}U^{x}ACAGUGCAAUGAGGGACCAGUACAUG^{x}A^{x}-[C6Amino dT]-3′ (SEQ ID NO:86), wherein ^{x} denotes a 2′-MOE. In some embodiments, the T-Oligo-HES conjugate contains an oligonucleotide having the sequence 5′-TUCAUGUACUGGUCCCUCAUUG CACUGUACU - 3′ (SEQ ID NO:87) (K-ras). In further embodiments, the T-Oligo-HES conjugate contains an oligonucleotide having the sequence 5′-[C6Amino dT]U^{x}C^{x}AUGUACUGGUC^{x}CCU^{x}CA^{x}UU^{x}G C^{X}AC^{X}UG^{X} UA^{x}C^{x}U^{x} - 3′ (SEQ ID NO:87), wherein ^{x} denotes a 2′-MOE. In some embodiments, the T-Oligo-HES conjugate contains a duplex oligonucleotide having the sequence 5′-AGUACAGUG CAAUGAGGGACCAGUACA UGAT-3′ (SEQ ID NO:86) and an oligonucleotide having the sequence 5′-TUCAUGUACUGGUCCCUCAUUGCACUG UACU- 3′ (SEQ ID NO:87).

In some embodiments, the T-Oligo-HES conjugate contains an oligonucleotide having the sequence 5′- AAGGCATCCCAGCCTCCGTT- 3′ (SEQ ID NO:82) (BCL2).

In some embodiments, the T-Oligo-HES conjugate contains an oligonucleotide having the sequence 5′- CCACAAAGGCATCCCAGCCTCCGTTATCCT - 3′ (SEQ ID NO:83) (BCL2).

In additional embodiments, the T-Oligo-HES conjugate contains an oligonucleotide that specifically hybridizes to a nucleic acid sequence that modulates apoptosis, cell survival, angiogenesis, metastasis, aberrant gene regulation, cell cycle, mitogenic pathways and/or growth signaling. In further embodiments, the T-Oligo-HES conjugate contains an oligonucleotide that specifically hybridizes to a nucleic acid sequence that modulates the expression of a protein selected from: EGFR, HER2/neu, ErbB3, cMet, p56lck, PDGFR, VEGF, VEGFR, FGF, FGFR, ANG1, ANG2, bFGF,TIE2, protein kinase C-alpha (PKC-alpha), p56lck PKA, TGF-beta, IGFIR, P12, MDM2, BRCA, IGF1, HGF, PDGF, IGFBP2, IGF1R, HIF1 alpha, ferritin, transferrin receptor, TMPRSS2, IRE, HSP27, HSP70, HSP90, MITF, clusterin, PARP1C-fos, C-myc, n-myc, C-raf, B-raf, A1, H-raf, Skp2, K-ras, N-ras, H-ras, farensyltransferase, c-Src, Jun, Fos, Bcr-Abl, c-Kit, EphA2, PDGFB, ARF, NOX1, NF1, STAT3, E6/E7, APC, WNT, beta catenin, GSK3b, PI3k, mTOR, Akt, PDK-1, CDK, Mek1, ERK1, AP-1, P53, Rb, Syk, osteopontin, CD44, MEK, MAPK, NF kappa beta, E cadherin, cyclin D, cyclin E, Bcl2, Bax, BXL-XL, BCL-W, MCL1, ER, MDR, telomerase, telomerase reverse transcriptase, a DNA methyltransferase, a histone deacetlyase (e.g., HDAC1 and HDAC2), an integrin, an IAP, an aurora kinase, a metalloprotease (e.g., MMP2, MMP3 and MMP9), a proteasome, and a metallothionein gene. In a particualr embodiment, the T-Oligo-HES conjugate contains an oligonucleotide that specifically hybridizes to a HER2 nucleic acid sequence. In a particualr embodiment, the T-Oligo-HES conjugate contains an oligonucleotide that specifically hybridizes to a P53 mutant nucleic acid sequence.

In additional embodiments, the T-Oligo-HES conjugate contains an oligonucleotide that specifically hybridizes to a nucleic acid sequence selected from the group: survivin, HSPB1, EIF4E, PTPN1, RRM2, BCL2, PTEN, Bcr-abl, TLR9, HaRas, Pka-rIA, JNK2, IGF1R, XIAP, TGF-β2, c-myb, PLK1, K-ras, KSP, PKN3, Ribonucleotide Reductase, Ribonucleotide Reductase R1, Ribonucleotide Reductase R2, MEM2 and TLR-9. In a particualr embodiment, the T-Oligo-HES conjugate contains an oligonucleotide that specifically hybridizes to a BCL2 nucleic acid sequence. In a particualr embodiment, the T-Oligo-HES conjugate contains an oligonucleotide that specifically hybridizes to a K-RAS nucleic acid sequence.

In some embodiments, the T-Oligo-HES conjugate contains an oligonucleotide that specifically hybridizes to a HIF1-alpha or HIF1-beta nucleic acid sequence.

In additional embodiments, the T-Oligo-HES conjugate contains an oligonucleotide that specifically hybridizes to a checkpoint inhibitor nucleic acid sequence. In some embodiments, the T-Oligo-HES conjugate contains an oligonucleotide that specifically hybridizes to a nucleic acid sequence selected from the group: PD1, PD-L1, PD-L2, CTLA4 LAG3, TIM-3, TIGIT, VISTA, B7-H3, BTLA, A2aR and CD73.

In an additional embodiment, the T-Oligo-HES conjugate contains an oligonucleotide that specifically hybridizes to JAK1/2 or STAT1 nucleic acid sequence.

In an additional embodiment, the T-Oligo-HES conjugate contains an oligonucleotide that specifically hybridizes to a nrp1 or nsp12 nucleic acid sequence.

In further embodiments, the T-Oligo-HES conjugate contains an oligonucleotide that specifically hybridizes to a nucleic acid sequence of a RecQ helicase family member. In particular embodiments, the RecQ helicase family member is Werner protein (WRN). In other embodiments, the RecQ helicase family member is RecQL1. In other embodiments, the RecQ helicase family member is a member from: BLM, RecQL4, RecQ5, and RTS. Exemplary GenBank accession references for the target nucleic acid sequences are: BLM at U39817, NM_000057, and BC034480; RecQ1 atNM_002907, NM _032941, BC001052, D37984, and L36140; WRN at NM_000553, AF091214, L76937, and AL833572; RecQ5 at NM_004259, AK075084, AB006533, AB042825, AB042824, AB042823, AF135183, and BC016911; and RTS at NM_004260, AB006532, BC020496, and BC011602 and BC013277.

In another embodiment, the disclosure provides a method of treating cancer or one or more conditions associated with cancer by administering a T-Oligo-HES conjugate in combination with one or more therapies currently being used, have been used, or are known to be useful in the treatment of cancer or conditions associated with cancer.

In some embodiments, the disclosure provides a method of treating an inflammatory or other disease or disorder of the immune system, or one or more conditions associated with an inflammatory or other disease or disorder of the immune system, said method comprising administering to a subject in need thereof (i.e., having or at risk of having an inflammatory or other immune system disease or disorder), a therapeutically effective amount of one or more T-Oligo-HES conjugates as provided herein. As immediately apparent to those skilled in the art, any type of immune or inflammatory disease or condition resulting from or associated with an immune system or inflammatory disease can be treated in accordance with the methods provided herein. In particular embodiments, the disclosure provides a method of treating disease or disorder of the immune system and/or an inflammatory disease or disorder, or one or more conditions associated with such disease or disorder.

The term “inflammatory disorders”, as used herein, refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders additionally include but are not limited to those affecting the blood vessels (polyarteritis, temporal arteritis); joints (arthritis: crystalline, osteo, psoriatic, reactive, rheumatoid, Reiter’s); gastrointestinal tract (Disease); skin (dermatitis); or multiple organs and tissues (systemic lupus erythematosus). The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis. In particular embodiments, the inflammatory disorder is selected from the group consisting of asthma, allergic disorders, and rheumatoid arthritis.

In further embodiment, the disorder or disorder of the immune system is an autoimmune disease. Autoimmune diseases, disorders or conditions that may be treated using the T-Oligo-HES conjugates provided herein include, but are not limited to, autoimmune hemolytic anemia, autoimmune neonatal thrombocytopenia, idiopathic thrombocytopenia purpura, autoimmune neutropenia, autoimmunocytopenia, hemolytic anemia, antiphospholipid syndrome, dermatitis, gluten-sensitive enteropathy, allergic encephalomyelitis, myocarditis, relapsing polychondritis, rheumatic heart disease, glomerulonephritis (e.g., IgA nephropathy), Multiple Sclerosis, Neuritis, Uveitis Ophthalmia, Polyendocrinopathies, Purpura (e.g., Henloch Scoenlein purpura), Reiter’s Disease, Stiff-Man Syndrome, Autoimmune Pulmonary Inflammation, myocarditis, IgA glomerulonephritis, dense deposit disease, rheumatic heart disease, Guillain-Barre Syndrome, insulin dependent diabetes mellitus, and autoimmune inflammatory eye, autoimmune thyroiditis, hypothyroidism (i.e., Hashimoto’s thyroiditis, systemic lupus erythematous, discoid lupus, Goodpasture’s syndrome, Pemphigus, Receptor autoimmunities for example, (a) Graves′ Disease, (b) Myasthenia Gravis, and (c) insulin resistance, autoimmune hemolytic anemia, autoimmune thrombocytopenic purpura, rheumatoid arthritis, scleroderma with anti-collagen antibodies, mixed connective tissue disease, polymyositis/dermatomyositis, pernicious anemia, idiopathic Addison’s disease, infertility, glomerulonephritis such as primary glomerulonephritis and IgA nephropathy, bullous pemphigoid, Sjogren’s syndrome, diabetes mellitus, and adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, asthma, inflammatory myopathies, and other inflammatory, granulomatous, degenerative, and atrophic disorders. In particular embodiments, the autoimmune disease or disorder is selected from Crohn’s disease, Systemic lupus erythematous (SLE), inflammatory bowel disease, psoriasis, diabetes, ulcerative colitis, multiple sclerosis, and rheumatoid arthritis.

In additional embodiments, the disclosure provides a method of treating an immune or cardiovascular disease comprising administering to a therapeutically effective amount of a T-Oligo-HES conjugate to a subject in need thereof. In particular embodiments, the T-Oligo-HES conjugate contains an oligonucleotide that binds a nucleic acid target selected from: ICAM-1, P53, TNF-α, Adenosine A1 receptor; PCSK9, SERPINC1, TFR2, TMPRSS6, CCR3, c-reactive protein (CRP), Apo-B100, ApoCIII, Apo(a), Apo(b), Factor VII and Factor XI.

In some embodiment, the disclosure provides a method of treating an immune or cardiovascular disease comprising administering a therapeutically effective amount of a T-Oligo-HES conjugate to a subject in need thereof. In particular embodiments, the T-Oligo-HES conjugate contains an oligonucleotide selected from: Alicaforsen (ICAM-1; ISIS 2302), QPI-1002 (P53; Silence Thera/Novartis/Quark), XEN701 (Isis/Xenon Pharmaceuticals), ISIS 104838 (TNF-α; ISIS/Orasense), EPI-2010 (RASON; Adenosine A1 receptor; Epigenesis/Genta), Plazomicin (Isis/Achaogen), ALN-PCS02 (PCSK9; Alnylam), ALN-AT3 (SERPINC1; Alnylam), ALN-HPN (TFR2; Alnylam), ALN-HPN (TMPRSS6; Alnylam), ASM8-003 (CCR3; Topigen Pharmaceuticals), ISIS CRPRx (CRP; ISIS), Kynamro™ (ISIS 301012; Apo-B100; ISIS/Genzyme), ISIS-APOCIII Rx (ApoCIII; ISIS), ISIS-APO(a) (Apo(a); ISIS); ISIS-FVII rx (Factor VII; ISIS), and ISIS-FXI (Factor XI; ISIS). In particular embodiments, the T-Oligo-HES conjugate provided herein contains an oligonucleotide that competes with one of the above oligonucleotides for target binding.

In additional embodiments, the T-Oligo-HES conjugate contains an oligonucleotide that binds ApoB. In additional embodiments, the T-Oligo-HES conjugate contains Mipomersen (ApoB). In particular embodiments, the T-Oligo-HES conjugate contains an oligonucleotide that competes with Mipomersen for target ApoB nucleic acid binding.

In further embodiment, the disclosure provides methods of treating an immune or cardiovascular disease comprising administering to a subject a therapeutically effective amount of a T-Oligo-HES conjugate. In particular embodiments, the T-Oligo-HES conjugate contains an oligonucleotide having a sequence selected from: 5′-GCCCAAGCTGGCAT CCGTCA-3′ (SEQ ID NO:19)(ICAM-1); 5′-GCTGATTAGAGAGAGGTCCC-3′ (SEQ ID NO:20)(TNF-α); and 5′-GATGGAGGGCGGCATGGCGGG-3′ (SEQ ID NO:21)(adenosine A1 receptor). In particular embodiments, the T-Oligo-HES conjugate contains an oligonucleotide that competes with one or more of the above oligonucleotides for target binding.

In some embodiments, the disclosure provides a method of treating an infectious disease or one or more conditions associated with an infectious disease, said method comprising administering to a subject in need thereof (i.e., having or at risk of having an infectious disease), a therapeutically effective amount of one or more conjugates comprising a T-Oligo-HES conjugate as provided herein. In some embodiments, the infectious disease is a viral infection, a bacterial infection, a fungal infection or a parasite infection.

In some embodiments, the disclosure provides a method of treating an infection or condition associated with a category A infectious agent or disease, said method comprising administering to a subject in need thereof (i.e., having or at risk of having an infectious disease), a therapeutically effective amount of one or more conjugates comprising a T-Oligo-HES conjugate as provided herein. In particular embodiments, the infectious agent is selected from Bacillus anthracis, Clostridium botulinum toxin, yersina pestis, variola major a filovirus (e.g., Ebola and Marburg) and an arenavirus (e.g., Lassa and Machupo). In particular embodiments, the treated condition is selected from: anthrax, botulism, plague, smallpox, tularemia, and a viral hemorrhagic fever.

In some embodiments, the disclosure provides a method of treating an infection or condition associated with a category B infectious agent or disease, said method comprising administering to a subject in need thereof (i.e., having or at risk of having an infectious disease), a therapeutically effective amount of one or more T-Oligo-HES conjugates provided herein. In particular embodiments, the infectious agent is selected from: a Bacilla species, Clostridium perfringens, a Salmonella species, E. coli 0157:H7, Shigella, Burkholderia pseudomallei, Chyamydia psittaci, Coxiella burnetii, Rickettsia prowazekii, a viral encephalitis alphavirus (e.g., Venezuelan equine encephalitis, eastern equine encephalitis, western equine encephalitis), Vibrio cholerae and Cryptosporidium parvum. In particular embodiments, the treated condition is selected from: Brucellosis, epsilon toxin of Clostridium perfringens, food poisoning, Glanders, Melioidosis, Psittacosis, Q fever, ricin toxin poisoning, typhus fever, viral encephalitis and dysentery.

In some embodiments, the disclosure provides a method of treating a viral infection or one or more conditions associated with a viral infection, said method comprising administering to a subject in need thereof (i.e., having or at risk of having a viral infection), a therapeutically effective amount of one or more of a T-Oligo-HES conjugates provided herein. As immediately apparent to those skilled in the art, any type of viral infection or condition resulting from or associated with a viral infection (e.g., a respiratory condition) can be treated in accordance with the methods provided herein. In particular embodiments, the viral disease or disorder is an infection or condition associated with a member selected from: Ebola, Marburg, Junin, Denge West Nile, Lassa SARS Co-V, Japanese encephalitis, Venezuelan equine encephalitis, Saint Louis encephalitis, Manchupo, Yellow fever, and Influenza.

Examples of viruses which cause viral infections and conditions that can be treated with the T-Oligo-HES conjugates provided herein include, but are not limited to, infections and conditions associated with retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), arenavirus (e.g., lassa fever virus), paramyxoviruses (e.g., morbillivirus virus, human respiratory syncytial virus, mumps, hMPV, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and PIV), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus).

In additional embodiments, the disclosure provides a method of treating or alleviating conditions associated with viral respiratory infections associated with or that cause the common cold, viral pharyngitis, viral laryngitis, viral croup, viral bronchitis, influenza, parainfluenza viral diseases (“PIV”) diseases (e.g., croup, bronchiolitis, bronchitis, pneumonia), respiratory syncytial virus (“RSV”) diseases, metapneumavirus diseases, and adenovirus diseases (e.g., febrile respiratory disease, croup, bronchitis, and pneumonia).

In some embodiments, the T-Oligo-HES conjugate contains an oligonucleotide selected from: AVI-4065 (HCV; AVI Biopharma), VRX496 (HIV; VIRxSYS corporation), Miravirsen (antimiR-122, Santaris), GEM 91 (Trecorvirsen)/92 (5′-CTCTCGCAC CCATCTCTCTCCTTCT-3′) (SEQ ID NO:22); Gag HIV; Hybridon), Vitravene (Fomivirsen; CMV; ISIS/Novartis (5′-GCGTTTGCTCTTCTTCTTGCG-3′) (SEQ ID NO:23)), ALN-RSV01 (RSV; Alnylam), AVI-6002 (Ebola; AVI Biopharma), AVI-6003 (Ebola; AVI Biopharma), MBI-1121 (human papillomavirus; Hybridon), ARC-520 (HPV hepatitis; Arrowhead Research Corporation) and AVI-6001 (Influenza/avian flu; AVI Biopharma). In some embodiment, the T-Oligo-HES conjugate contains an oligonucleotide selected from: ISIS 14803 (HCV; ISIS (5′-GTGCTCATGGTGCACGGTCT-3′) (SEQ ID NO:24)) and 5′-TCGTCGTTTTGTCGTTTTGTCGTT-3′ (SEQ ID NO:25) (CMV). In particular embodiments, the oligonucleotide in conjugate competes for target nucleic acid binding with one of the above oligonucleotides.

In one embodiment, the disclosure provides a method of treating an RSV infection or one or more conditions associated with an RSV infection by administering to a subject in need thereof, a T-Oligo-HES conjugate containing an oligonucleotide that binds to at least 1, at least 2, at least 3, at least 4, at least 5, at least 6, at least 7, at least 8, at least 9 or at least 10 RSV oligonucleotide sequences. In particular embodiments, the oligonucleotide-HES has a siRNA/Dicer sequence pair selected from the group consisting of: RSV-N oligonucleotides 5′-GGC UCUUAGCAAAGUCAAGUUGAAUGAU-3′ (SEQ ID NO:26) and 5′-AUCA UUCAACUUGACUUUGCUAAGAGCCAU-3′ (SEQ ID NO:27); RSV-P oligonucleotides 5′-CGAUAAUAUAACUGCAAGAdTdT-3′ (SEQ ID NO:28) and 3′-dTdTGCUAUUAU AUUGACGUUCU-5′ (SEQ ID NO:29); and RSV-F oligonucleotides 5′-UGCUGUA ACAGAAUUGCAGdTdT-3′ (SEQ ID NO:30) and 5′-CUGCAAUUCUGUUACAGCad TdT-3′ (SEQ ID NO:31). In particular embodiments, the oligonucleotide in conjugate competes for target nucleic acid binding with one of the above oligonucleotides. In further embodiments, one or more of the oligonucleotides in the conjugate is a PMO or a PPMO. In additional embodiments, one or more of the oligonucleotides in the conjugate is an antisense, a siRNA or a shRNA.

In an additional embodiment, the disclosure provides a method of treating a viral infection or one or more conditions associated with a viral infection by administering a combination of at least 1, at least 2, at least 3, at least 4, or at least 5 T-Oligo-HES conjugates to a subject in need thereof. In some embodiments, at least 2, at least 3, or at least 4 of the oligonucleotides in the conjugates specifically hybridizes to the same target nucleic acid. In additional embodiments, at least 2, at least 3, or at least 4 or at least 5 of the oligonucleotides in the conjugates specifically hybridize to a different target nucleic acid.

In one embodiment, the disclosure provides a method of treating a filovirus (e.g., Ebola and Marbury) infection or one or more conditions associated with the infection by administering to a patient in need thereof, a therapeutically effective amount of a T-Oligo-HES conjugate containing an oligonucleotide that specifically hybridizes to at least 1, at least 2, at least 3, at least 4, at least 5, at least 6, at least 7, at least 8, at least 9 or at least 10 RNA sequences of a filovirus. In particular embodiments, the oligonucleotide of the conjugate binds VP35, VP24 and/or RNA polymerase L. In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating an Ebola virus infection or one or more conditions associated with the infection by administering to a patient in need thereof, a T-Oligo-HES conjugate containing an oligonucleotide that binds to at least 1, at least 2, at least 3, at least 4, at least 5, at least 6, at least 7, at least 8, at least 9 or at least 10 Ebola RNA sequences. In particular embodiments, the oligonucleotide of the conjugate binds VP24, VP35, and/or RNA polymerase L. In additional embodiments, the oligonucleotide of the conjugate binds VP24, VP30, VP35, VP40, NP, GP and/or RNA polymerase L. In particular embodiments, the oligonucleotide of the conjugate binds VP35 and have an antisense sequence of 5′-6CCTGCCCTT TGTTCTAGTTG 6-3′ (SEQ ID NO:32; wherein C6 refers to a C6 linker arm attached to the base moiety of Uridine and G6 refers to a G6 linker arm attached to the base moiety of Uridine). In additional embodiments, the oligonucleotide of the conjugate binds VP35 and have a siRNA/Dicer sequence pair selected from the group consisting of: 5′-GCGACAUCUUCUGUGAUAUUG-3′ (SEQ ID NO:33) and 5′-AUAU CACAGAAGAUGUCGCUU-3′ (SEQ ID NO:34); 5-CAUUACGAGUCU UGAGAAU-3′ (SEQ ID NO:35) and 5′-UCUCAAGACUCGUAAUGCG-3′ (SEQ ID NO:36); 5′-GCAAC UCAUUGGACAUCAUUC-3′ (SEQ ID NO:37) and 5′-AUGAUGUCCAAUGAGUU GCUA-3′ (SEQ ID NO:38); 5′-UGAUGAAGAUUAAGAAAAA-3′ (SEQ ID NO:39) and 5′-UUUCUUAAUCUUCAUCACU-3′ (SEQ ID NO:40); 5′-GUG CUGAGAUGGUUGCAAA-3′ (SEQ ID NO:41) and 5′-UGCAACCAUCUCA GCACAA-3′ (SEQ ID NO:42); 5′-GCU AAUGACCGGAAGAAUU-3′ (SEQ ID NO:43) and 5′-UUCUUCCGGUCA UUAGCUG-3′ (SEQ ID NO:44); and 5′-CCAAUUAGUACAAGUGAUU-3′ (SEQ ID NO:45) and 5′-UCACUUGUACUAAUUGGUG-3′ (SEQ ID NO:46). In particular embodiments, the oligonucleotide of the conjugate binds NP and have an antisense sequence selected from the group consisting of: 5′-6GAGAATCCATACTCGGAATT6-3′ (SEQ ID NO:47); 5′-6GACG AGAATCCATACTCGGA6-3′ (SEQ ID NO:48); and 5′-6GCATGTACTTGAATTTGCC6-3′ (SEQ ID NO:49; wherein “6” refers to a C6 linker arm attached to the base moiety of Uridine). In additional embodiments, the oligonucleotide of the conjugate binds NP and have a siRNA/Dicer sequence pair selected from the group consisting of: 5′-GGCAAAUUCA AGUACA UGCdTdT-3′ (SEQ ID NO:50) and 5′-GCAUGUACUUGAAUUUGCCUU (SEQ ID NO:51); 5′-GCAUGGAGAGUAUGCUCCUUU-3′ (SEQ ID NO:52) and 5′-AGGAGCAUACUCUCCAUGCUU (SEQ ID NO:53); 5′- ATGGTGATTTTCCGTT TGAT-3′ (SEQ ID NO:54) and 5′- TCAAACGGAAAATCACCAT-3′ (SEQ ID NO:55); and 5′-GAGAAGCAACTCCAACAAT-3′ (SEQ ID NO:56) and 5′-UGUUGGAGUUG CUUCUC-3′ (SEQ ID NO:57). In particular embodiments, the oligonucleotide of the conjugate binds RNA polymerase L and have an antisense sequence of 5′-6TGGGTATGTTGTGTAGCCAT6-3′ (SEQ ID NO:58); In additional embodiments, the oligonucleotide of the conjugate binds RNA polymerase L and have a siRNA/Dicer sequence pair selected from the group consisting of: 5′-GUACGAAGCUGUAUAUAAAUU-3′ (SEQ ID NO:59) and 5′-UUUAUAUACAGCUUCG UACUU-3′ (SEQ ID NO:60). In particular embodiments, the oligonucleotide of the conjugate binds VP24 and have an antisense sequence of 5′-6GCCATGGTTTTTTCTCAGG6-3′ (SEQ ID NO:61). In additional embodiments, the oligonucleotide of the conjugate binds VP24 and have a siRNA/Dicer sequence pair selected from the group consisting of: 5′-GCUGAUUGACCAGUCUUUGAU-3′ (SEQ ID NO:62) and 5′-CAAAGACUGGUCAAUCAGCUG-3′ (SEQ ID NO:63); 5′-ACGGAUUGUUGAGCAGUAUUG-3′ (SEQ ID NO:64) and 5′-AUACUGCUCAACAAU CCGUUG-3′ (SEQ ID NO:65); and 5′-UCCUCGACACGAAUGCAAAGU-3′ (SEQ ID NO:66) and 5′-UUUGCAUUCGUGUCGAGGAUC-3′ (SEQ ID NO:67). In particular embodiments, the oligonucleotide in conjugate competes for target nucleic acid binding with one of the above oligonucleotides. In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating an Flaviviridae (e.g., West Nile, yellow fever, Japanese encephalitis, and dengue viruses) viral infection or one or more conditions associated with the infection by administering to a patient in need thereof, a therapeutically effective amount of a T-Oligo-HES conjugate containing an oligonucleotide that specifically hybridizes to at least 1, at least 2, at least 3, at least 4, or at least 5 RNA sequences of a member of the family Flaviviridae. In particular embodiments, the oligonucleotide of the conjugate binds the highly conserved non coding sequence in the 5′ or 3′ regions of the viral genome, or sequence corresponding to the envelope coding gene (E). In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating an Arenavirideae (e.g., Lassa, Junin and Machupo viruses) family viral infection or one or more conditions associated with the infection by administering to a patient in need thereof, a therapeutically effective amount of a T-Oligo-HES conjugate containing an oligonucleotide that specifically hybridizes to at least 1, at least 2, at least 3, at least 4, or at least 5 RNA sequences of a member of the family Arenavirideae. In particular embodiments, the oligonucleotide of the conjugate binds the highly conserved non coding sequence in the 5′ or 3′ viral mRNAs transcript coding for the Z protein (zinc-binding protein), L protein (viral polymerase), or the GPC (glycoprotein precursor) protein. In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating a SARS-associated coronavirus (SARS Co-V) infection or one or more conditions associated with the infection by administering to a patient in need thereof, a therapeutically effective amount of a T-Oligo-HES conjugate containing an oligonucleotide that specifically hybridize to at least 1, at least 2, at least 3, at least 4, or at least 5 family SARS Co-V nucleic acid sequences. In particular embodiments, the oligonucleotide of the conjugate binds the replica se gene (orf 1a/1b), orf 1b ribosomal frameshift point, 5′ untranslated region (UTR) of the transcription regulatory sequence (TRS), 3′ UTR of the TRS sequence, spike protein-coding region and/or the NSP12 region. In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating an Retroviridae (e.g., HIV viruses) family viral infection or one or more conditions associated with the infection by administering to a patient in need thereof, a therapeutically effective amount of a T-Oligo-HES conjugate containing an oligonucleotide that specifically hybridizes to at least 2, at least 3, at least 4, or at least 5 RNA sequences of a member of the family Retroviridae. In particular embodiments, the oligonucleotide of the conjugate binds the highly conserved regions of the gag, pol, int, and Vpu regions. In further embodiments, one or more of the oligonucleotides in the conjugate is a PMO or a PPMO. In additional embodiments, one or more of the oligos in the conjugate is an antisense, a siRNA or a shRNA.

In another embodiment, the disclosure provides a method of treating an influenza A (e.g., H1N1, H3N2 and H5N1) infection or one or more conditions associated with influenza by administering to a patient in need thereof, a therapeutically effective amount of a T-Oligo-HES conjugate containing an oligonucleotide that specifically hybridizes to at least 2, at least 3, at least 4, or at least 5 influenza RNA sequences. In particular embodiments, the oligonucleotide of the conjugate binds NP and PA nucleic acid sequence of the virus. In particular embodiments, the oligonucleotide of the conjugate binds an NP, M2, and/or PB2 (e.g., targeting the AUG start codon of PA, PB1, PB2, and NP), or terminal region of NP), NS1 and/or PA nucleic acid sequence of the virus. In further embodiments, one or more of the oligonucleotides in the conjugate is a PMO or a PPMO. In additional embodiments, one or more of the oligonucleotides in the conjugate is an antisense, a siRNA or a shRNA.

In one embodiment, the disclosure provides a method of treating an influenza virus infection or one or more conditions associated with the infection by administering to a patient in need thereof, a T-Oligo-HES conjugate wherein the oligonucleotide(s) in the conjugate specifically bind to at least 1, at least 2, at least 3, at least 4, at least 5, at least 6, at least 7, at least 8, at least 9 or at least 10 influenza RNA sequences. In particular embodiments, the oligonucleotide has a siRNA/Dicer sequence pair selected from the group consisting of: NP oligonucleotides 5′-GGAUCUUAUUUCUUCGGAG-3′ (SEQ ID NO:68) and 5′-CUCCGAA GAAAUAAGAUCC-3′ (SEQ ID NO:69); PA oligonucleotides 5′-GCAAUUGA GGAGUG CCUGA-3′ (SEQ ID NO:70) and 5′-UCAGCGACUCCUCAAUUGC-3′ (SEQ ID NO:71); PB1 oligonucleotides 5′-GAUCUGUUCCACCAUUGAA-3′ (SEQ ID NO:72) and 5′-UUCA AUGGUGGAACAGAUC-3′ (SEQ ID NO:73); and M2 oligonucleotides 5′-ACAGC AGA AUGCUGUGGAU-3′ (SEQ ID NO:74) and 5′-AUCCACAGCAUUCUGC UGU-3′ (SEQ ID NO:75). In particular embodiments, one or more oligonucleotide in the conjugate competes for target nucleic acid binding with one of the above oligonucleotides. In further embodiments, one or more oligonucleotides in the conjugate is a PMO or a PPMO. In additional embodiments, one or more oligonucleotides in the conjugate is an antisense, a siRNA or a shRNA.

In an additional embodiment, the disclosure provides a method of treating an alphavirus (equine encephalitis virus (VEEV)) infection or one or more conditions associated with an alphavirus infection by administering to a patient in need thereof, a therapeutically effective amount a conjugate comprising a targeting moiety conjugated to a T-Oligo-HES conjugate wherein the oligonucleotide specifically hybridizes to at least 2, at least 3, at least 4, or at least 5 alphavirus RNA sequences. In particular embodiments, the oligonucleotide of the conjugate binds NP and PA nucleic acid sequence of the virus. In particular embodiments, the oligonucleotide of the conjugate binds an nsp1, nsp4 and/or E1 RNA sequence of the virus. In further embodiments, one or more of the oligonucleotides in the conjugate is a PMO or a PPMO. In additional embodiments, one or more of the oligonucleotides in the conjugate is an antisense, a siRNA or a shRNA.

In some embodiments, the disclosure provides a method of treating a bacterial infection or one or more conditions associated with a bacterial infection, said method comprising administering to a subject in need thereof (i.e., having or at risk of having a bacterial infection), a therapeutically effective amount of one or more T-Oligo-HES conjugates provided herein. Any type of bacterial infection or condition resulting from, or associated with a bacterial infection can be treated using the compositions and methods provided herein. In particular embodiments, the bacterial infection or condition treated according to the methods provided herein is associated with a member of a bacterial genus selected from: Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia and Rochlimeae. In further embodiments, the treated bacterial infection or condition is associated with a member of a bacterial genus selected from: P. aeruginosa; E. coli, P. cepacia, S. epidermis, E. faecalis, S. pneumonias, S. aureus, N. meningitidis, S. pyogenes, Pasteurella multocida, Treponema pallidum, and P. mirabilis. In some embodiments, the bacterial infection is an intracellular bacterial infection. In additional embodiments, the disclosure provides a method of treating an bacterial infection or one or more conditions associated with a bacterial infection by administering to a patient in need thereof, a therapeutically effective amount a conjugate comprising a targeting moiety conjugated to a T-Oligo-HES conjugate wherein the oligonucleotide specifically hybridizes to at least 1, at least 2, at least 3, at least 4, or at least 5 nucleic acid sequences of at least 1, at least 2, at least 3, at least 4, or at least 5 of the above bacteria.

In additional embodiments, the disclosure provides a method of treating a fungal infection or one or more conditions associated with a fungal infection, said method comprising administering to a subject in need thereof (i.e., having or at risk of having a fungal infection), a therapeutically effective amount of one or more conjugates comprising a targeting moiety conjugated to a T-Oligo-HES conjugate as provided herein. Any type of fungal infection or condition resulting from or associated with a fungal infection can be treated using the compositions and methods provided herein. In particular embodiments, the fungal infection or condition treated according to the methods provided herein is associated with a fungus selected from: Cryptococcus neoformans; Blastomyces dermatitidis; Aiellomyces dermatitidis; Histoplasma capsulatum; Coccidioides immitis; a Candida species, including C. albicans, C. tropicalis, C. parapsilosis, C. guilliermondii and C. krusei, an Aspergillus species, including A. fumigatus, A. flavus and A. niger; a Rhizopus species; a Rhizomucor species; a Cunninghammella species; a Apophysomyces species, including A. saksenaea, A. mucor and A. absidia; Sporothrix schenckii, Paracoccidioides brasiliensis; Pseudalleseheria boydii, Torulopsis glabrata; a Trichophyton species, a Microsporum species and a Dermatophyres species, or any other fungus (e.g., yeast) known or identified to be pathogenic. In additional embodiments, the disclosure provides a method of treating a fungal infection or condition associated with a fungal infection by administering to a patient in need thereof, a therapeutically effective amount a conjugate comprising a targeting moiety conjugated to a T-Oligo-HES conjugate, wherein the oligonucleotide specifically hybridizes to at least 1, at least 2, at least 3, at least 4, or at least 5 nucleic acid sequences of at least 1, at least 2, at least 3, at least 4, or at least 5 of the above funghi.

In additional embodiments, the disclosure provides a method of treating a parasite infection or one or more conditions associated with a parasite infection, said method comprising administering to a subject in need thereof (i.e., having or at risk of having a parasite infection), a therapeutically effective amount of one or more conjugates comprising a targeting moiety conjugated to a T-Oligo-HES conjugate as provided herein. Any type of parasite infection or condition resulting from or associated with a parasite infection can be treated using the compositions and methods provided herein. In particular embodiments, the parasite infection or condition treated according to the methods provided herein is associated with a parasite selected from: a member of the Apicomplexa phylum such as, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus or Gregarina spp.; Pneumocystis carinii; a member of the Microspora phylum such as, Nosema, Enterocytozoon, Encephalitozoon, Septata, Mrazekia, Amblyospora, Arneson, Glugea, Pleistophora and Microsporidium spp.; and a member of the Ascetospora phylum such as, Haplosporidium spp. In further embodiments, the treated parasite infection or condition is associated with a parasite species selected from: Plasmodium falciparum, P. vivax, P. ovale, P. malaria; Toxoplasma gondii; Leishmania mexicana, L. tropica, L. major, L. aethiopica, L. donovani, Trypanosoma cruzi, T. brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T. saginata, Trichomonas vaginatis, T. hominis, T. tenax; Giardia lamblia; Cryptosporidium parvum; Pneumocytis carinii, Babesia bovis, B. divergens, B. microti, Isospora belli, L. hominis; Dientamoeba fragilis; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite known or identified to be pathogenic. In additional embodiments, the disclosure provides a method of treating an parasite infection or one or more conditions associated with a parasite infection by administering to a patient in need thereof, a therapeutically effective amount a conjugate comprising a targeting moiety conjugated to a T-Oligo-HES conjugate wherein the oligonucleotide specifically hybridizes to at least 1, at least 2, at least 3, at least 4, or at least 5 nucleic acid sequences of at least 1, at least 2, at least 3, at least 4, or at least 5 of the above parasites.

In another embodiment, the disclosure provides a method of treating a viral infection or one or more conditions associated with a viral infection by administering a T-Oligo-HES conjugate provided herein in combination with one or more therapies currently being used, have been used, or are known to be useful in the treatment of a viral infection or conditions associated with a viral infection, including but not limited to, anti-viral agents such as amantadine, oseltamivir, ribaviran, palivizumab, and anamivir. In certain embodiments, a therapeutically effective amount of one or more conjugates comprising a targeting moiety conjugated to a T-Oligo-HES conjugate as provided herein is administered in combination with one or more anti-viral agents such as, but not limited to, amantadine, rimantadine, oseltamivir, znamivir, ribaviran, RSV-IVIG (i.e., intravenous immune globulin infusion) (RESPIGAM™), and palivizumab.

In some embodiments, the disclosure provides a method of treating an respiratory disease or one or more conditions associated with a respiratory disease, said method comprising administering to a subject in need thereof (i.e., having or at risk of having an respiratory disease), a therapeutically effective amount of one or more conjugates comprising a targeting moiety conjugated to a T-Oligo-HES conjugate as provided herein. The term “respiratory disease,” as used herein, refers to a disease affecting organs involved in breathing, such as the nose, throat, larynx, trachea, bronchi, and lungs. Respiratory diseases that can be treated according to the methods provided herein include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

In some embodiments, the disclosure provides a method of treating a respiratory disease or one or more conditions associated with a respiratory disease comprising administering to a subject in need thereof a therapeutically effective amount of a T-Oligo-HES conjugate. In particular embodiments, the T-Oligo-HES conjugate contains an oligonucleotide that binds a nucleic acid selected from STK, RSV nucleocapsid, Akt1, WT1, IGF-1R, NUPR, PKN3, PI3K, NFKb, MMP-12, VEGF, CCR1, CCR3, IL8R, IL4R, caspase 3, IKK2, Syk, Lyn, STAT1, STAT6, GATA3, EZH2, let7, miR-34, miR-29, miR-223/1274a, miR1, miR-146a, miR-150, miR-21, miR-126, miR-155, miR-133a, let7d, miR-29, miR-200, miR-10a, miR-34, miR-123, miR-145, miR-150, miR-199b, miR-218 and miR-222.

In some embodiments, the disclosure provides a method of treating a metabolic disorder comprising administering to a subject a therapeutically effective amount of a T-Oligo-HES conjugate. In particular embodiments, the T-Oligo-HES conjugate contains an oligonucleotide that binds a nucleic acid selected from selected from: Exellair (Syk kinase) and ALN-RSV01 (RSV nucleocapsid). In particular embodiments, the T-Oligo-HES conjugate contains an oligonucleotide that competes with one of the above oligonucleotides for target binding.

In some embodiments, the disclosure provides a method of treating a neurological condition or disorder, comprising administering to a subject in need thereof (i.e., having or at risk of having a neurological condition or disorder), a therapeutically effective amount of a T-Oligo-HES conjugate provided herein. The term “neurological condition or disorder” is used herein to refer to conditions that include neurodegenerative conditions, neuronal cell or tissue injuries characterized by dysfunction of the central or peripheral nervous system or by necrosis and/or apoptosis of neuronal cells or tissue, and neuronal cell or tissue damage associated with trophic factor deprivation. Examples of neurodegenerative diseases that can be treated using the T-Oligo-HES conjugates provided herein include, but are not limited to, familial and sporadic amyotrophic lateral sclerosis (FALS and ALS, respectively), familial and sporadic Parkinson’s disease, Huntington’s disease (Huntington’s chorea), familial and sporadic Alzheimer’s disease, Spinal Muscular Atrophy (SMA), optical neuropathies such as glaucoma or associated disease involving retinal degeneration, diabetic neuropathy, or macular degeneration, hearing loss due to degeneration of inner ear sensory cells or neurons, epilepsy, Bell’s palsy, frontotemporal dementia with parkinsonism linked to chromosome 17 (FTDP-17), multiple sclerosis, diffuse cerebral cortical atrophy, Lewy-body dementia, Pick disease, trinucleotide repeat disease, prion disorder, and Shy-Drager syndrome. In some embodiments, the treated neurodegenerative disease is selected from familial and sporadic amyotrophic lateral sclerosis (FALS and ALS, respectively), familial and sporadic Parkinson’s disease, Huntington’s disease (Huntington’s chorea), familial and sporadic Alzheimer’s disease, Spinal Muscular Atrophy (SMA), multiple sclerosis, diffuse cerebral cortical atrophy, dementia, or Pick disease Examples of neuronal cell or tissue injuries that can be treating using T-Oligo-HES conjugates provided herein include, but are not limited to, acute and non-acute injury found after blunt or surgical trauma (including post-surgical cognitive dysfunction and spinal cord or brain stem injury) and ischemic conditions restricting (temporarily or permanently) blood flow such as that associated with global and focal cerebral ischemia (stroke); incisions or cuts for instance to cerebral tissue or spinal cord; lesions or placques in neuronal tissues; deprivation of trophic factor(s) needed for growth and survival of cells; and exposure to neurotoxins such as chemotherapeutic agents; as well as incidental to other disease states such as chronic metabolic diseases such as diabetes and renal dysfunction.

In some embodiments, the disclosure provides a method of treating a neurological condition or disorder comprising administering to a subject in need thereof, a therapeutically effective amount of a T-Oligo-HES conjugate. In some embodiments, the T-Oligo-HES conjugate contains an oligonucleotide that binds a DMD nucleic acid sequence. In particular embodiments, the T-Oligo-HES conjugate contains an oligonucleotide selected from: AVI-4658 (Dystrophin (exon-skipping); AVI Biopharma), ISIS-SMN Rx (SMN; ISIS/ Biogen Idec), AVI-5126 (CABG; AVI Biopharma) and ATL1102 (VLA-4 (CD49d); ISIS/Antisense Therapeutics Ltd). In additional embodiments, the T-Oligo-HES conjugate contains Eteplirsen or Drisapersen. In particular embodiments, the T-Oligo-HES conjugate contains an oligonucleotide that competes with one of the above oligonucleotides for target binding.

In some embodiments, the disclosure provides a method of treating a metabolic disorder comprising administering a therapeutically effective amount of a T-Oligo-HES conjugate oligonucleotide-HES to a subject in need thereof. In particular embodiments, the T-Oligo-HES conjugate contains an oligonucleotide that binds a nucleic acid selected from: FGFR4, GCC, PTP1VB, DME, TTR, PTPN1, DGAT and AAT. In particular embodiments, the T-Oligo-HES conjugate contains an oligonucleotide that binds a nucleic acid selected from: ISIS-FGFR4 (FGFR4; ISIS), ISIS-GCCR RX (GCC; ISIS), ISIS-GCGRRX (GCG; ISIS), ISIS-PTP1B (PTP1VB; ISIS (5′-GCTCC TTCCACTGATCCTGC-3′)(SEQ ID NO:76)), iCo-007 (c-Raf; Isis/iCo Therapeutics Inc. (5′-TCCCGCCTGTGACATGCATT-3′)(SEQ ID NO:6)); ISIS-DGATRX (DGAT; ISIS), PF-04523655 (DME; Silence Thera/Pfizer/Quark), ISIS-TTR Rx (TTR; ISIS/GSK); ISIS-AAT Rx (AAT; ISIS/GSK), ALN-TTRsc (Transerythrin; Alnylam), ALN-TTR01 (Transerythrin; Alnylam), and ALN-TTR02 (Transerythrin; Alnylam). In particular embodiments, the T-Oligo-HES conjugate contains an oligonucleotide that competes with one of the above oligonucleotides for target binding.

In some embodiment, the disclosure provides a method of treating a disease comprising administering a therapeutically effective amount of a T-Oligo-HES conjugate to a subject in need thereof.

In some embodiments, the T-Oligo-HES conjugate contains an oligonucleotide that binds a target nucleic acid, and a targeting moiety that specifically binds a cell surface antigen on the cell or in the microenvironment of the cell containing the targeted nucleic acid. In some embodiments, the T-Oligo-HES conjugate contains an oligonucleotide that binds a target nucleic acid selected from: GHr, CTGF and PKN3. In particular embodiments, the T-Oligo-HES conjugate contains an oligonucleotide selected from: ATL1103-GHr Rx (GHr; ISIS/Antisense Therapeutics Ltd), EXC 001 (CTGF; ISIS/Excaliard), and Atu111 (PKN3; Silence Thera). In particular embodiments, the T-Oligo-HES conjugate contains an oligonucleotide that competes with one of the above oligonucleotides for target binding.

In addition to those described above, T-Oligo-HES conjugates provided herein have applications including but not limited to; treating metabolic diseases or disorders (e.g., mellitus, obesity, high cholesterol, high triglycerides), in treating diseases and disorder of the skeletal system (e.g., osteoporosis and osteoarthritis), in treating diseases and disorders of the cardiovascular system (e.g., stroke, heart disease, atherosclerosis, restenosis, thrombosis, anemia, leucopenia, neutropenia, thrombocytopenia, granuloctopenia, pancytoia or idiopathic thrombocytopenic purpura); in treating diseases and disorders of the kidneys (e.g., nephropathy), pancreas (e.g., pancreatitis), skin and eyes (e.g., conjunctivitis, retinitis, scleritis, uveitis, allergic conjuctivitis, vernal conjunctivitis, pappillary conjunctivitis glaucoma, retinopathy, and ocular ischemic conditions including anterior ischemic optic neuropathy, age-related macular degeneration (AMD), Ischemic Optic Neuropathy (ION), dry eye syndrome); in preventing organ transplantation rejection (e.g., lung, liver, heart, pancreas, and kidney transplantation) and uses in regenerative medicine (e.g., in counteracting aging, in promoting wound healing and stimulating bone, collagen, tissue and organ growth and repair).

In some embodiment the disclosure provides a method of treating of treating a disease comprising administering to a subject in need thereof, a therapeutically effective amount of a T-Oligo-HES conjugate containing an oligonucleotide that binds a target nucleic acid selected from: P53, caspase 2, keratin 6a, adrenergic receptor beta 2, VEGFR1, RTP801, ApoB and VEGF. In particular embodiments, the T-Oligo-HES conjugate contains an oligonucleotide selected from: TKM-ApoB (ApoB), I5NP (P53), QPI-1007 (caspase 2), TD101 (keratin 6a), SYL040012 (adrenergic receptor beta 2), AGN-745 (VEGFR1), PF-655 (RTP801), and Bevasiranib (VEGF). In particular embodiments, the T-Oligo-HES conjugate contains an oligonucleotide that competes with one of the above oligonucleotides for target binding.

In various embodiments, the disclosure provides compositions for use in modulating a target nucleic acid or protein in a cell, in vivo in a subject, or ex vivo. The T-Oligo-HES conjugates provided herein have applications in for example, treating a disease or disorder characterized by an overexpression, underexpression and/or aberrant expression of a nucleic acid or protein in a subject in vivo or ex vivo. Uses of the T-Oligo-HES conjugates in treating exemplary diseases or disorders selected from: an infectious disease, cancer, a proliferative disease or disorder, a neurological disease or disorder, and inflammatory disease or disorder, a disease or disorder of the immune system, a disease or disorder of the cardiovascular system, a metabolic disease or disorder, a disease or disorder of the skeletal system, and a disease or disorder of the skin or eyes are also provided herein. In a particular aspect, the T-Oligo-HES conjugates provided herein are used to treat a metastasis.

As one of skill in the art will immediately appreciate, the therapeutic and companion diagnostic uses of conjugates comprising a targeting moiety conjugated to a T-Oligo-HES conjugate are essentially limitless. Provided herein are exemplary diagnostic and therapeutic uses of the conjugates. However, the description herein is not meant to be limiting and it is envisioned that the conjugates have uses in any situations where it is desirable to detect a nucleic acid sequence or to modulate levels of one or more nucleic acids or related proteins in a cell and/or organism.

### Plurality of T-Oligo-HES Conjugates

In some embodiments, the conjugates provided herein comprise a combination of at least 2, at least 3, at least 4, at least 5, or at least 10 different oligonucleotides. In some embodiments, the disclosure provides pharmaceutical compositions comprising a plurality of conjugates which collectively contain at least 2, at least 3, at least 4, at least 5, or at least 10 different oligonucleotides or T-Oligo-HES conjugates having different oligonucleotide sequences. In some embodiments, the pharmaceutical composition contains between 2-15, 2-10, or 2-5 different oligonucleotides and/or T-Oligo-HES conjugate es. In some embodiments, at least 2 or at least 3 of the different oligonucleotides in the complex specifically hybridize to a DNA and/or mRNA corresponding to the same polypeptide. In some embodiments, at least 2, at least 3, at least 4, at least 5, or at least 10 of the different oligonucleotides in the complex specifically hybridizes to a DNA and/or mRNA corresponding to different polypeptides. In some embodiments, the pharmaceutical compositions contain between 2-15, 2-10, or 2-5 oligonucleotides that specifically hybridize to different polypeptides. In some embodiments, one or more of the different T-Oligo-HES conjugates are administered to a subject concurrently. In other embodiments, one or more of the different T-Oligo-HES conjugates are administered to a subject separately.

In certain embodiments, a T-Oligo-HES conjugate provided herein is co-administered with one or more additional agents. In certain embodiments, such additional agents are designed to treat a different disease, disorder, or condition as the T-Oligo-HES conjugate. In some embodiments, the additional agent is co-administered with the T-Oligo-HES conjugate to treat an undesired effect of the complex. In additional embodiments, the additional agent is co-administered with the T-Oligo-HES conjugate to produce a combinational effect. In further embodiments, the additional agent is co-administered with the T-Oligo-HES conjugate to produce a synergistic effect. In certain embodiments, the additional agent is administered to treat an undesired side effect of a T-Oligo-HES conjugate. In some embodiments, the T-Oligo-HES conjugate is administered at the same time as the additional agent. In some embodiments, the oligonucleotide-HES and additional agent are prepared together in a single pharmaceutical formulation. In other embodiments, the oligonucleotide-HES and additional agent are prepared separately. In further embodiments, the additional agent is administered at a different time from the T-Oligo-HES conjugate.

### Kits

The disclosure also provides kits for the administration of the T-Oligo-HES conjugates described herein. The kit is an assemblage of materials or components, including at least one of the T-Oligo-HES conjugates described herein. Thus, in some embodiments, the kit contains at least one of the T-Oligo-HES conjugates.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to affect a desired outcome, such as to treat cancer, inflammation, autoimmune disease, or a neurodegenerative disease. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Methods of Treatment

Methods and compositions described herein have application to treating various diseases and disorders, including, but not limited to cancer, infections, immune disorders, anemia, autoimmune diseases, cardiovascular diseases, wound healing, ischemia-related diseases, neurodegenerative diseases, metabolic diseases and many other diseases and disorders.

Further, any of the present agents may be for use in the treating, or the manufacture of a medicament for treating, various diseases and disorders, including, but not limited to cancer, infections, immune disorders, inflammatory diseases or conditions, and autoimmune diseases.

In some embodiments, the disclosure relates to the treatment of, or a patient having one or more of cancer, heart failure, autoimmune disease, sickle cell disease, thalassemia, blood loss, transfusion reaction, diabetes, vitamin B12 deficiency, collagen vascular disease, Shwachman syndrome, thrombocytopenic purpura, Celiac disease, endocrine deficiency state such as hypothyroidism or Addison’s disease, autoimmune disease such as Crohn’s Disease, systemic lupus erythematosus, rheumatoid arthritis or juvenile rheumatoid arthritis, ulcerative colitis immune disorders such as eosinophilic fasciitis, hypoimmunoglobulinemia, or thymoma/thymic carcinoma, graft versus host disease, preleukemia, Nonhematologic syndrome (e.g., Down’s, Dubowwitz, Seckel), Felty syndrome, hemolytic uremic syndrome, myelodysplasic syndrome, nocturnal paroxysmal hemoglobinuria, osteomyelofibrosis, pancytopenia, pure red-cell aplasia, Schoenlein-Henoch purpura, malaria, protein starvation, menorrhagia, systemic sclerosis, liver cirrhosis, hypometabolic states, and congestive heart failure.

In some embodiments, the disclosure relates to the treatment of, or a patient having cancer. As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated using T-Oligo-HES conjugates provided herein include, but are not limited to, carcinomas, e.g., various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g., gliomas (e.g., astrocytoma, oligodendroglioma, and ependymoma), meningioma, pituitary adenoma, and neuromas, and spinal cord tumors (e.g., meningiomas and neurofibroma).

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin’s and non-Hodgkin’s lymphoma, as well as B-cell lymphoma (including low grade/follicular non-Hodgkin’s lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom’s Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs’ syndrome.

In some embodiments, T-Oligo-HES conjugates provided herein are used to treat a patient having a microbial infection and/or chronic infection. Illustrative infections include, but are not limited to, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In various embodiments, T-Oligo-HES conjugates provided herein are used to treat or prevent one or more inflammatory diseases or conditions, such as inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses.

In various embodiments, T-Oligo-HES conjugates provided herein are used to treat or prevent one or more autoimmune diseases or conditions, such as multiple sclerosis, diabetes mellitus, lupus, celiac disease, Crohn’s disease, ulcerative colitis, Guillain-Barre syndrome, scleroderms, Goodpasture’s syndrome, Wegener’s granulomatosis, autoimmune epilepsy, Rasmussen’s encephalitis, Primary biliary sclerosis, Sclerosing cholangitis, Autoimmune hepatitis, Addison’s disease, Hashimoto’s thyroiditis, Fibromyalgia, Menier’s syndrome; transplantation rejection (e.g., prevention of allograft rejection) pernicious anemia, rheumatoid arthritis, systemic lupus erythematosus, dermatomyositis, Sjogren’s syndrome, lupus erythematosus, multiple sclerosis, myasthenia gravis, Reiter’s syndrome, Grave’s disease, and other autoimmune diseases.

In various embodiments, T-Oligo-HES conjugates provided herein are used to treat, control or prevent cardiovascular disease, such as a disease or condition affecting the heart and vasculature, including but not limited to, coronary heart disease (CHD), cerebrovascular disease (CVD), aortic stenosis, peripheral vascular disease, atherosclerosis, arteriosclerosis, myocardial infarction (heart attack), cerebrovascular diseases (stroke), transient ischemic attacks (TIA), angina (stable and unstable), atrial fibrillation, arrhythmia, vavular disease, and/or congestive heart failure.

In various embodiments, T-Oligo-HES conjugates provided herein are used to treat or prevent one or more metabolic-related disorders. In various embodiments, T-Oligo-HES conjugates are useful for the treatment, controlling or prevention of diabetes, including Type 1 and Type 2 diabetes.

In various embodiments, T-Oligo-HES conjugates provided herein are used to treat or prevent one or more respiratory diseases, such as asthma, chronic obstructive pulmonary disease (COPD), bronchiectasis, allergic rhinitis, sinusitis, pulmonary vasoconstriction, inflammation, allergies, impeded respiration, respiratory distress syndrome, cystic fibrosis, pulmonary hypertension, pulmonary vasoconstriction, emphysema, Hantavirus pulmonary syndrome (HPS), Loeffler’s syndrome, Goodpasture’s syndrome, Pleurisy, pneumonitis, pulmonary edema, pulmonary fibrosis, Sarcoidosis, complications associated with respiratory syncytial virus infection, and other respiratory diseases.

In some embodiments, T-Oligo-HES conjugates provided herein are used to treat or prevent one or more neurodegenerative disease. Illustrative neurodegenerative disease include, but are not limited to, multiple sclerosis (including without limitation, benign multiple sclerosis; relapsing-remitting multiple sclerosis (RRMS); secondary progressive multiple sclerosis (SPMS); progressive relapsing multiple sclerosis (PRMS); and primary progressive multiple sclerosis (PPMS)), Alzheimer’s. disease (including, without limitation, Early-onset Alzheimer’s, Late-onset Alzheimer’s, and Familial Alzheimer’s disease (FAD), Parkinson’s disease and parkinsonism (including, without limitation, Idiopathic Parkinson’s disease, Vascular parkinsonism, Drug-induced parkinsonism, Dementia with Lewy bodies, Inherited Parkinson’s, Juvenile Parkinson’s), Huntington’s disease, Amyotrophic lateral sclerosis (ALS, including, without limitation, Sporadic ALS, Familial ALS, Western Pacific ALS, Juvenile ALS, Hiramaya Disease).

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. In addition, the term ‘cell’ can be construed as a cell population, which can be either heterogeneous or homogeneous in nature, and can also refer to an aggregate of cells. Moreover, each of the limitations of the invention can encompass various embodiments of the invention. It is, therefore, envisioned that each of the limitations of the invention involving any one element or combinations of elements can be included in each embodiment of the invention.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention.

The disclosure of each of U.S. Appl. No. 62/959,928, Intl. Appl. No. PCT/US2014/42202, and Intl. Appl. No. PCT/US2012/069294, is herein incorporated by reference in its entirety.

Moreover, all publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

The following examples are offered to illustrate, but not to limit, the claimed invention.

### Example 1

Exemplary synthetic scheme T (antibody)-oligo-HES conjugates. T -oligo-HES conjugates are conjugates composed of four components: a targeting moiety such as an antibody, a linker, a conformation-specific peptide, and an oligonucleotide-HES complex. In this example the targeting moiety was an antibody. The disulfides in the antibody’s Fc domain were first reduced with Tris(2-Carboxyethyl)-Phosphine (TCEP) followed by addition of a maleimide on one end of the linker. The linker arm typically ranges between six and fifty atoms, in this example the linker arm had 25 atoms plus a dibenzocyclooctyne. Using click chemistry a peptide containing a conformation-specific cleavage site (as described in Example 3) with an azide on one end was then conjugated to the linker on the antibody. The peptide containing a conformation-specific cleavage site was then able to serve as a cleavage site for a protease on a cell surface (see examples below). The peptide can then be conjugated on the side distal to the antibody linkage to an oligonucleotide, either a single strand antisense or a double strand siRNA (in this case a single strand antisense sequence with a sequence complementary to k-RAS). Both the peptide containing a conformation-specific cleavage site and the oligonucleotide, each previously derivatized with two fluorophore such that they form an HES, specifically, a peptide HES and an oligonucleotide HES, are then able to deliver a conjugate to the cell surface such that the antibody can bind to the antigen on the cell surface (in this case rituximab), the conformation-specific peptide can be cleaved by the cognate protease (in this case a matrix metalloprotease), the oligonucleotide HES then diffuses into the cell so that it can hybridize with its complementary sequence (in this case k-RAS).

### Example 2

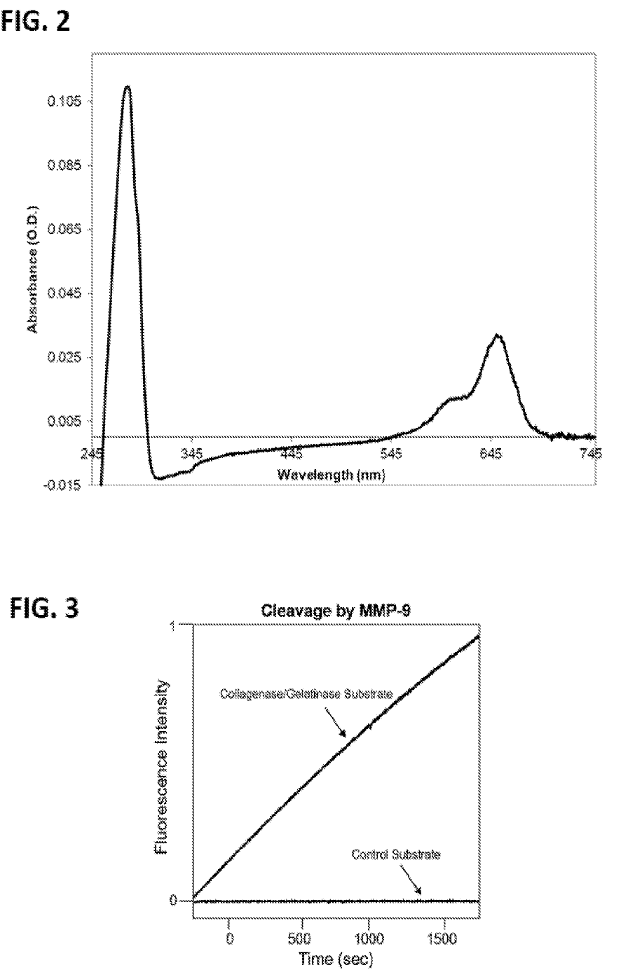

An antibody covalently labeled with a linker arm which can then be derivatized with a fluorophore through click chemistry to confirm the presence of the arm. An antibody was first reduced with TCEP followed by addition of a linker with 25 atoms which included four ethyleneglycol groups. The linker which terminated with a dibenzocyclooctyne group was then conjugated with an azide-bearing fluorophore. After a 1 hour reaction time, the solution was passed over a gel filtration (BioGel P30) column with the conjugate eluting immediately after the void volume. As indicated in FIG. 2, the peak at 280 nm is due to the antibody and the peak at 641 indicates the covalent attachment of the fluorophore. The complete synthesis of T (e.g., antibody)-oligo-HES conjugates can occur in multiple steps such as, for example, (a) by adding the linker first and then sequentially adding a peptide containing a conformation-dependent cleavage site and an oligonucleotide or (b) by adding the linker already conjugated to the peptide containing the conformation-dependent cleavage site and oligonucleotide directly to the antibody. Each step can be checked by using complementary functional chemical groups containing reporter groups such as fluorophores.

### Example 3

Specificity of a peptide containing a conformation-specific cleavage site. An eighteen amino acid peptide containing the amino acid sequence PLGIA (SEQ ID NO:78) was covalently labeled with the same fluorophore near each end such that an intramolecular H-type excitonic dimer formed giving rise to an HES structure. The PLGIA sequence was recognized by matrix metalloprotease -9 (MMP-9) and is cleaved between the LG and the IA. MMP-19 is added in a pH 7.5 buffer in which the PLGIA (SEQ ID NO:77) peptide is at 2 uM. Cleavage of the peptide gives rise to an increase in fluorescence intensity. This specificity is compared with a control peptide, i.e., an HES-bearing peptide of the same length, with the same labeling, but that does not contain the conformation-dependent cleavage site. The fluorescence of the latter does not increase upon addition of the MMP indicating specificity of the PLGIA (SEQ ID NO:77) sequence for MMP-19. See, FIG. 3.

### Example 4

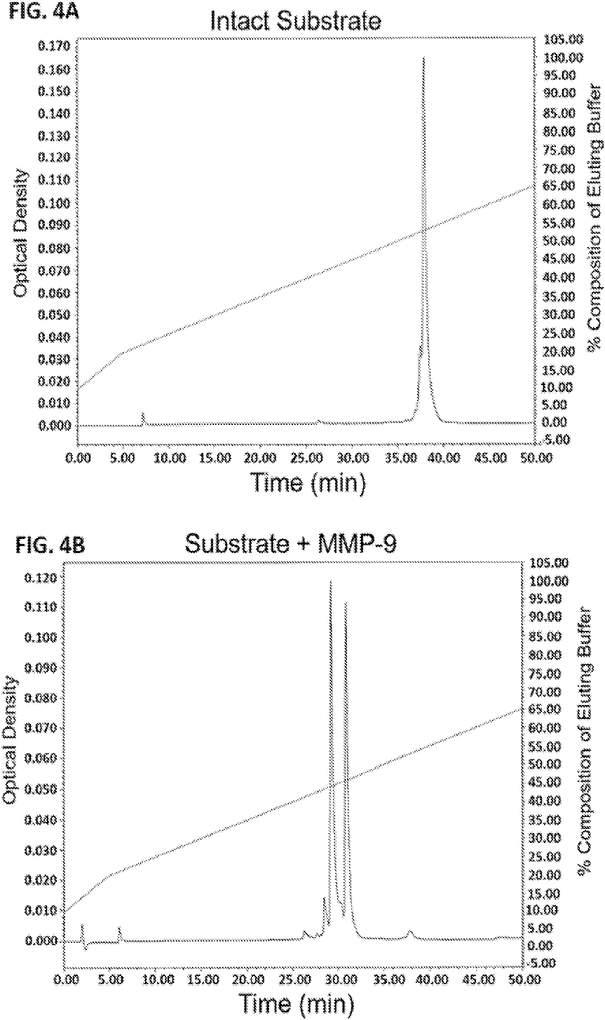

The retention time of the HES-PLGIA (SEQ ID NO:77) peptide containing a conformation-dependent cleavage site was determined by HPLC where the retention time on a C18 column was determined under reverse phase conditions, i.e., loading in an aqueous buffer and eluting in an acetonitrile buffer, to be 38 minutes (see, FIG. 4A). After exposure to MMP-9, the major peaks were at ca. 30 and 31 minutes with the almost complete disappearance of the 38 minute peak (FIG. 4B), consistent with the cleavage as indicated in Example 3. See, e.g., FIG. 3.

### Example 5

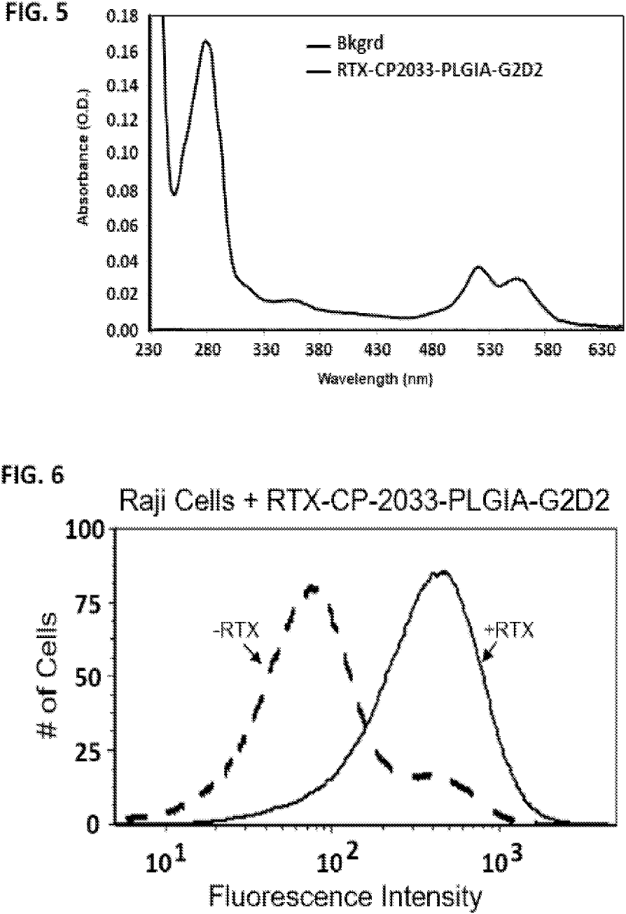

Formation of an antibody linked to a peptide containing a conformation-dependent cleavage site. Rituximab, a monoclonal antibody which recognizes CD20 on B-lymphocytes, was conjugated to a linker and a peptide containing a conformation-dependent cleavage site. The peak at 280 nm indicates the presence of the antibody. The conformation-dependent specificity of the peptide cleavage site which is due to the presence of the HES is indicated by the more intense peak at 520 nm relative to that at 552 nm. If the two fluorophores which form the intramolecular H-dimer were not present, then the peak at 552 would be higher than that at 520. Thus, the conformational specificity of the peptide is maintained after covalent bond formation with the linker. See, e.g., FIG. 5.

### Example 6

Recognition of rituximab-oligo-HES conjugate by B cells. Raji cells, a CD20+ B lymphocyte cell line, was exposed to rituximab labeled with a linker arm and a peptide containing a conformation-dependent cleavage site at 4° C. After washing and addition of a viability dye, the cells were examined by flow cytometry. Cells exposed to the modified rituximab conjugate recognized the antibody and bound it with no effect on the cells′ viability. Thus, modification of the antibody by the chemistry of addition by click chemistry did not diminish the recognition function of the monoclonal antibody. See, e.g., FIG. 6.

What is claimed is:

1. A conjugate comprising a targeting moiety conjugated to an oligonucleotide-HES complex, optionally wherein the oligonucleotide is a therapeutic oligonucleotide.

2. The conjugate according to claim 1, wherein the targeting moiety is directly conjugated to the oligonucleotide-HES complex or is conjugated to the oligonucleotide-HES complex through a linker.

3. The conjugate of claim 1 or 2, wherein the targeting moiety is conjugated to the oligonucleotide-HES complex through a linker.

4. The conjugate of any one of claims 1 to 3 having the structure of formula (I) 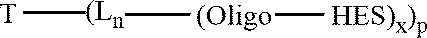
wherein:
T is a targeting moiety that selectively binds a target of interest;
L is a linker;
Oligo-HES is an oligonucleotide complex containing a therapeutic oligonucleotide and an H-type excitonic structure (HES);
n is 0 or 1;
x is 1 to 30, 1-20, 1-10, or 1-5; and
p is 1 to 30, 1-20, 1-10, or 1-5.

5. The conjugate of any one of claims 1 to 4 having the structure of formula (II) 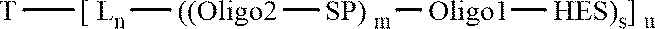
wherein:
T is a targeting moiety that selectively binds a target of interest;
L is a linker;
SP is a linker, optionally wherein SP is 6 to 12 amino acid residue peptide or alkyl chain spacer such as a C6, C10, or C18, linear or branched alkyl;
Oligo1-HES is an oligonucleotide complex containing oligonucleotide 1 (Oligo1) and an H-type excitonic structure (HES);
Oligo2 is an oligonucleotide that may be the same or different from Oligo1;
n is 0 or 1;
m is 0 or 1;
s is 1 or 2; and
u is 1, 2, 3, 4, or 5.

6. The conjugate of any one of claims 1 to 5, wherein the oligonucleotide-HES complex comprises a therapeutic oligonucleotide that specifically hybridizes to a nucleic acid sequence in vivo and modulates the level of a protein encoded or regulated by the nucleic acid.

7. The conjugate of claim 6, wherein the therapeutic oligonucleotide contains 1, 2, or 3 substitutions, deletions, or insertions, compared to the corresponding reverse complementary strand of the nucleic acid sequence.

8. The conjugate of any one of claims 1 to 7, wherein the therapeutic oligonucleotide is from about 8 nucleotides to about 750 nucleotides in length.

9. The conjugate of any one of claims 1 to 8, wherein the therapeutic oligonucleotide is 18-25, 18-35, 18-40, or 18-45, 18- 50, 18-60, 18- 70, 18-80, 18- 90, 18-100, 18- 150, or 18-200 nucleotides in length.

10. The conjugate of any one of claims 1 to 9, wherein the therapeutic oligonucleotide is single stranded.

11. The conjugate of any one of claims 1 to 9, wherein the therapeutic oligonucleotide is double stranded.

12. The conjugate of claim 11, wherein the therapeutic oligonucleotide is 36-50, 36-60, 36-70, or 36-100 nucleotides in length.

13. The conjugate of any one of claims 1 to 12, wherein the therapeutic oligonucleotide contains one or more modified nucleoside motifs selected from: locked nucleic add (LNA), alpha LNA, 2′-Fluoro (2′F), 2′-O(CH2)2OCH3 (2′-MOE), 2′--deoxy-2′--fluoro-D-arabinonucleic acids (FANA), 2′-OCH3 (2′-O-methyl) (2′OME), PNA, and morpholino.

14. The conjugate of claim 13, wherein the modified nucleoside motif is an LNA or alpha LNA in which a methylene (--CH2--)n group bridges the 2′ oxygen atom and the 4′ carbon atom wherein n is 1 or 2.

15. The conjugate of claim 13 or 14, wherein the LNA or alpha LNA contains a methyl group at the 5′ position.

16. The conjugate of any one of claims 1 to 15, that contains one or more modified internucleoside linkages selected from: phosphorothioate, phosphorodithioate, phosphoramide, 3′-methylene phosphonate, O-methylphosphoroamidiate, PNA and morpholino.

17. The conjugate of any one of claims 1 to 16, wherein the therapeutic oligonucleotide contains one or more modified nucleobases selected from C-5 propyne, and 5-methyl C.

18. The method according to any one of claims 1 to 17, wherein the therapeutic oligonucleotide of the conjugate specifically hybridizes to a nucleic acid selected from: EGFR, HER2/neu, ErbB3, cMet, p56lck, PDGFR, VEGF, VEGFR, FGF, FGFR, ANG1, ANG2, bFGF,TIE2, protein kinase C-alpha (PKC-alpha), p56lck PKA, TGF-beta, IGFIR, P12, MDM2, BRCA, IGF1, HGF, PDGF, IGFBP2, IGF1R, HIF1 alpha, ferritin, transferrin receptor, TMPRSS2, IRE, HSP27, HSP70, HSP90, MITF, clusterin, PARP1C-fos, C-myc, n-myc, C-raf, B-raf, A1, H-raf, Skp2, K-ras, N-ras, H-ras, farensyltransferase, c-Src, Jun, Fos, Bcr-Abl, c-Kit, EphA2, PDGFB, ARF, NOX1, NF1, STAT3, E6/E7, APC, WNT, beta catenin, GSK3b, PI3k, mTOR, Akt, PDK-1, CDK, Mek1, ERK1, AP-1, P53, Rb, Syk, osteopontin, CD44, MEK, MAPK, NF kappa beta, E cadherin, cyclin D, cyclin E, Bcl2, Bax, BXL-XL, BCL-W, MCL1, ER, MDR, telomerase, telomerase reverse transcriptase, a DNA methyltransferase, a histone deacetlyase (e.g., HDAC1 and HDAC2), an integrin, an IAP, an aurora kinase, a metalloprotease (e.g., MMP2, MMP3 and MMP9), a proteasome, or a metallothionein gene.

19. The method according to any one of claims 1 to 17, wherein the therapeutic oligonucleotide of the conjugate specifically hybridizes to a nucleic acid selected from: survivin, HSPB1, EIF4E, PTPN1, RRM2, BCL2, PTEN, Bcr-abl, TLR9, HaRas, Pka-rIA, JNK2, IGF1R, XIAP, TGF-β2, c-myb, PLK1, K-ras, KSP, PKN3, a Ribonucleotide Reductase (e.g., Ribonucleotide Reductase R1 and Ribonucleotide Reductase R2), a RecQ helicase (e.g., WRN, RecQL1, BLM, RecQL4, RecQ5, and RTS), MEM2 and TLR9.

20. The conjugate of any one of claims 1 to 19, wherein the oligonucleotide-HES complex comprises at least 1 fluorophore with an excitation and/or emission from 300-850 nm.

21. The conjugate of any one of claims 1 to 20, wherein the oligonucleotide-HES complex comprises 2, 3, 4, or more fluorophores capable of forming one or more HES.

22. The conjugate of any one of claims 1 to 21, wherein the oligonucleotide-HES complex comprises 2, 3, 4, or more fluorophores with an excitation and/or emission from 300-850 nm.

23. The conjugate of any one of claims 1 to 22, wherein the oligonucleotide-HES complex comprises at least 1 fluorophore selected from a xanthene, an indocarbocyanine, an indodicarbocyanine, and a coumarin.

24. The conjugate of any one of claims 1 to 23, wherein the oligonucleotide-HES complex comprises at least 1 fluorophore selected from: carboxyrhodamine 110, carboxytetramethylrhodamine, carboxyrhodamine-X, diethylaminocoumarin and an N-ethyl-N′-[5-(N″-succinimidyloxycarbonyl)pentyl]indocarbocyanine chloride, N-ethyl-N′-[5-(N″-succinimidyloxycarbonyl)pentyl]-3,3,3′,3′-tetramethyl-2′,2′-indodicarbocyanine chloride dye.

25. The conjugate of any one of claims 1 to 24, wherein the therapeutic oligonucleotide is selected from: siRNA, shRNA, miRNA, an antagmir, a Dicer substrate, and an antisense.

26. The conjugate of any one of claims 1 to 25, wherein the therapeutic oligonucleotide is a siRNA.

27. The conjugate of any one of claims 1 to 25, wherein the therapeutic oligonucleotide is a shRNA.

28. The conjugate of any one of claims 1 to 25, wherein the therapeutic oligonucleotide is a miRNA or an antagmir (inhibitor of miRNA).

29. The conjugate of any one of claims 1 to 25, wherein the therapeutic oligonucleotide is a Dicer substrate.

30. The conjugate of claims 1 to 29, wherein the therapeutic oligonucleotide contains 2 nucleic complementary nucleic acid strands that are each 18-25, 18-30, 18-35, 18-40, 18-45, or 18-50, nucleotides in length and a 2 nucleotide 3′ overhang.

31. The conjugate of any one of claims 1 to 30, wherein the therapeutic oligonucleotide can induce RNA interference (RNAi).

32. The conjugate of claim 25, wherein the therapeutic oligonucleotide is a substrate for RNAse H when hybridized to the RNA.

33. The conjugate of claim 25, wherein the therapeutic oligonucleotide is a gapmer.

34. The conjugate of claim 25, wherein the therapeutic oligonucleotide is not a substrate for RNAse H when hybridized to the RNA.

35. The conjugate of any one of claims 1 to 25, wherein the therapeutic oligonucleotide is an antisense oligonucleotide.

36. The conjugate of claim 35, wherein the antisense oligonucleotide specifically hybridizes to an RNA of interest.

37. The conjugate of claim 35 or 36, wherein the antisense oligonucleotide is DNA or a DNA mimic.

38. The conjugate of any one of claims 35 to 37, wherein each nucleoside of the therapeutic antisense oligonucleotide comprises a modified sugar moiety comprising a modification at the 2′-position, a PNA motif, or a morpholino motif.

39. The conjugate of any one of claims 35 to 38, wherein, the therapeutic antisense oligonucleotide sequence specifically hybridizes to a target region of an RNA selected from the group consisting of:
(a) a sequence within 30 nucleotides of the AUG start codon of an mRNA;
(b) nucleotides 1-10 of a miRNA;
(c) a sequence in the 5′ untranslated region of an mRNA;
(d) a sequence in the 3′ untranslated region of an mRNA;
(e) an intron/exon junction of an mRNA;
(f) a sequence in a precursor-miRNA (pre-miRNA) or primary-miRNA (pri-miRNA) that when bound by the oligonucleotide blocks miRNA processing; and
(g) an intron/exon junction and a region 1 to 50 nucleobases 5′ of an intron/exon junction of an RNA.

40. The conjugate of any one of claims 35 to 39, wherein hybridization of the oligonucleotide to its target mRNA sterically blocks translation of the coding sequences or modulates expression of the target mRNA, and/or blocks a nucleotide binding protein and thereby modulates target mRNA stability.

41. The conjugate of any one of claims 35 to 40, wherein the antisense oligonucleotide-HES complex comprises a plurality of antisense strands, optionally wherein the antisense strands are cross-linked.

42. The conjugate of claim 41, wherein the oligonucleotide-HES complex comprises a plurality of antisense oligonucleotides linked in linear with a spacer arm in series 5′ to 3′ and linked with a spacer arm (e.g., a 6 to 30 amino acid residue peptide or a linear alkyl (e.g., C6, C10, or C12) or a polyethyloxy-glycol (e.g., triethyloxy-glycol, tetraethyloxy-glycol or hexa-ethyloxy-glycol) to another antisense oligonucleotide linked with 2nd strand 5′ end or 3′ terminal residue.

43. The conjugate of any one of claims 1 to 42, wherein with linker arm (L or SP) is a linear alkyl of C6, C10, or C12, or a polyethyloxy-glycol (e.g., a triethyloxy-glycol, tetraethyloxy-glycol or hexa-ethyloxy-glycol).

44. The conjugate of any one of claims 41 to 43, wherein the Oligo-HES complex comprises a plurality of therapeutic antisense cross-linked with 2 or more antisense strands 5′ to 3′ and 2 or more antisense strands 3′ to 5′ in opposite orientation, Oligo-HES complex comprises a plurality of therapeutic antisense cross-linked with 2 or more antisense strands in 5′ to 3′ with a spacer SP then 3′ to 5′ orientation.

45. The conjugate of claim 44, wherein the antisense strands have 2 or more of the same complementary sequence and/or 2 or more different complementary sequences.

46. The conjugate of any one of claims 1 to 45, which comprises a linear or branched linker.

47. The conjugate of any one of claims 1 to 46, which comprises a peptide linker or SP spacer of 6 to 30 amino acid residues in length.

48. The conjugate of claim 46 or 47, wherein the linker is a linear peptide of 6 to 30 amino acid residues in length.

49. The conjugate of any one of claims 46 to 48, wherein the linker is cleavable.

50. The conjugate of claim 49, wherein the linker has an amino acid sequence comprising a protease cleavage site containing P1-P1′ residues and having a looped conformation.

51. The conjugate of claim 49 or 50, wherein the cleavable linker comprises an amino acid sequence that is a substrate for at least one protease.

52. The conjugate of any one claims 49 to 51, wherein the cleavable linker comprises an amino acid sequence that is a substrate for at least one protease that is active in a diseased tissue.

53. The conjugate of any one of claims 49 to 52, wherein the cleavable linker comprises an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

54. The conjugate of any one of claims 49 to 53, wherein the cleavable linker comprises an amino acid sequence that is a substrate for at least one protease selected from:
(a) MMP9;
(b) MMP14;
(c) MMP1, MMP2, MMP3, MMP7, MMP8, MMP10, MMP11, MMP12, MMP13, MMP15, MMP16, MMP17, MMP19, MMP20, MMP23, MMP24, MMP26, and MMP27;
(d) a serine protease (e.g., MT-SP1, uPA, and TMPRSS2);
(e) a cysteine protease;
(f) a metalloprotease including (a)-(c);
(g) an aspartyl protease; and
(h) a threonine protease.

55. The conjugate of claim 54, wherein the cleavable linker comprises an amino acid sequence that is a substrate for MMP9 or MMP14.

56. The conjugate of claim 54 or 55, wherein the cleavable linker comprises an amino acid sequence that is a substrate for at least one enzyme of the immune complement system such as u-plasminogen activator, tissue plasminogen activator, trypsin, or plasmin.

57. The conjugate of any one of claims 49 to 56, wherein the cleavable linker comprises an amino acid sequence that is a substrate for a protease known or reported to be colocalized with the target of the conjugate.

58. The conjugate of claim 49, wherein the linker is cleavable under intracellular conditions (e.g., conditions within a lysosome or endosome or caveolae).

59. The conjugate of claim 49, wherein the linker is pH-sensitive.

60. The conjugate of claim 49, wherein the linker is cleavable under reducing conditions.

61. The conjugate of conjugate of any of claims 44 to 56, wherein the linker is a malonate linker.

62. The conjugate of any of claims 46 to 48, wherein the linker is non-cleavable.

63. The conjugate of any one of claims 46 to 62, wherein the linker has an H-dimer forming fluorophore (e.g., a linker comprising an H-dimer forming fluorophore conjugated at the amino and/or carboxyl terminal residues);.

64. The conjugate of any one of claims 46 to 63, wherein the linker has an H-dimer forming fluorophore conjugated at the amino and carboxyl terminal residues.

65. The conjugate of any one of claims 46 to 64, wherein the linker has a sulfhydryl or amino functional group at the amino and carboxyl terminus of the peptide.

66. The conjugate of any one of claims 1 to 65, wherein the targeting moiety of the conjugate is an aptamer, avimer, a receptor-binding ligand, a nucleic acid, a biotin-avidin binding pair, a peptide, protein a carbohydrate, lipid, vitamin, a component of a microorganism, a hormone, a receptor ligand (including Fc fusion proteins containing the same), an antibody, an antigen binding portion of an antibody, an alternative binding scaffold, or any derivative thereof.

67. The conjugate of any one of claims 1 to 66, wherein the targeting moiety is an antibody, an antigen binding portion of an antibody (e.g., a Fab, and a scFv), or a single-domain antibody.

68. The conjugate of any one of claims 1 to 67, wherein the targeting moiety is an antibody, a humanized antibody, an antigen binding fragment of an antibody, a single chain antibody, a bi-specific antibody, a synthetic antibody, or a PEGylated antibody.

69. The conjugate of any one of claims 1 to 68, wherein the targeting moiety is an antibody.

70. The conjugate of claim 69, wherein the targeting moiety is a monospecific, bispecific or multispecific antibody and/or a monovalent, bivalent, or multivalent antibody.

71. The conjugate of claim 69 or 70, wherein the targeting moiety is an IgG1, IgG2, or IgG4 antibody.

72. The conjugate of any one of claims 69 to 70, wherein the targeting moiety is a therapeutic antibody.

73. The conjugate of claim 72, wherein the antibody is selected from: trastuzumab (HER2/neu), pertuzumab (HER2/neu), panitumumab (EGFR), nimotuzumab (EGFR), zalutumumab (EGFR), cetuximab (EGFR), (HER3), onartuzumab (c-MET), patritumab, clivatuzumab (MUC1), sofituzumab (MUC16), edrecolomab, (EPCAM), adecatumumab (EPCAM), anetumab (MSLN), huDS6 (CA6), lifastuzumab (NAPI2B), sacituzumab (TROP2), PR1A3, humanized PR1A3 (CEA), humanized Ab 2-3 (CEA), IMAB362/claudiximab (Claudin18.2), AMG595 (EGFRvIII), ABT806 (EGFRvIII), sibrotuzumab (FAP), DS-8895a variant 1 (EphA2), DS-8895a variant 2 (EphA2), anti-EphA2 (EphA2), MEDI-547 (EphA2), narnatumab (RON), RG7841 (LY6E), farletuzumab (FRA /folate receptor alpha), mirvetuximab (FRA), J591 variant 1 (PSMA), J591 variant 2 (PSMA), rovalpituzumab (DLL3), PF-06647020 (PTK7), anti-PTK7 (PTK7), ladiratuzumab (LIV1), cirmtuzumab (ROR1), rituximab (CD20), ibritumomab tiuxetan (CD52), alemtuzumab (CD33), Gemtuzumab ozogamicin (CD33), CT-011 (PD1), tositumomab (CD20), ipilimumab (CTLA4), tremelimumab (CP-675,206)(CTLA4), nivolumab (PD1), and pembrolizumab (PD1), durvalumab (PDL1) anti-MAGE-A3, anti-NY-ESO-1, anti-ACE2, anti-hyaluronidase, or anti-neuraminidase.

74. The conjugate of any one of claims 1 to 68, wherein the targeting moiety is an antigen binding fragment of an antibody, a single chain antibody, a single-domain antibody, or a bi-specific antibody.

75. The conjugate of any one of claims 1 to 66, wherein the targeting moiety is an alternative binding scaffold selected from: an affibody, nanobody, anticalin, fynomer, DARPin, Tetranectin, Transbody, AdNectin, Affilin, Microbody, peptide aptamer, alterase, plastic antibody, phylomer, stradobody, maxibody, evibody, Z domain, D domain, armadillo repeat protein, Kunitz domain, avimer, atrimer, probody, immunobody, triomab, troybody, pepbody, vaccibody, UniBody, Affimer, or a DuoBody.

76. The conjugate of any one of claims 1 to 75, wherein the targeting moiety specifically binds a cell surface antigen on or near a cell or tissue of interest such as, a diseased cell, a cancer cell, an immune cell, an infected cell, or an infectious agent.

77. The conjugate of any one of claims 1 to 76, wherein the targeting moiety specifically binds a cell surface antigen(s) derived, from or determined to be expressed on, a specific subject’s cancer (e.g., tumor) such as a neoantigen.

78. The conjugate of any one of claims 1 to 77, wherein the targeting moiety specifically binds a cell surface antigen that does not internalize the conjugate upon binding.

79. The conjugate of any one of claims 1 to 75, wherein the targeting moiety specifically binds a cell surface antigen that internalizes the conjugate upon binding.

80. The conjugate of any one of claims 1 to 79, wherein the targeting moiety specifically binds a tumor cell surface antigen.

81. The conjugate of any one of claims 1 to 80, wherein the targeting moiety specifically binds a tumor cell surface antigen a leukemic cell, lymphoma cell, pancreatic cancer cell, breast cancer cell, melanoma cell, lung cancer cell, head and neck cancer cell, ovarian cancer cell, bladder cancer cell, colon cancer cell, kidney cancer cell, liver cancer cell, prostate cancer cell, bone cancer cell, or brain cancer cell including a glioblastoma cell; or any lymphoma, myeloma, blastoma, sarcoma, leukemia or carcinoma cell.

82. The conjugate of any one of claims 1 to 81, wherein the targeting moiety specifically binds a cell surface antigen selected from: CD5, CD19, CD20, CD25, CD37, CD30, CD33, CD45, CD204, CD206, CD301, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Ley, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFRvIII, HER2/neu, MAGE A3, P53 nonmutant, NY-ESO-1, MelanA/MART1, Ras mutant, gp100, P53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PDL, VTCN1, and VISTA.

83. The conjugate of any one of claims 1 to 81, wherein the targeting moiety specifically binds a cell surface antigen selected from: HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15.

84. The conjugate of any one of claims 1 to 83, wherein the targeting moiety specifically binds a tumor microenvironment cell surface antigen (including certain membrane anchored proteases).

85. The conjugate of any one of claims 1 to 84, wherein the targeting moiety specifically binds a cell surface antigen expressed on endothelial cells or macrophages (e.g., VEGFR, TIE1, and TIE2), or tumor stromal cells such as cancer-associated fibroblasts (CAFs), and tumor infiltrating T cells and other leukocytes, and myeloid cells including mast cells, eosinophils, and tumor-associated macrophages.

86. The conjugate of any one of claims 1 to 85, wherein the targeting moiety specifically binds a cell surface antigen on an immune cell.

87. The conjugate of claim 86, wherein the targeting moiety specifically binds a cell surface antigen on an immune cell of lymphoid or myeloid origin such as a T cell, a B cell, an NK cell, an NKT cell, or a dendritic cell.

88. The conjugate of claim 86 or 87, wherein the targeting moiety specifically binds a cell surface antigen on an antigen presenting cell.

89. The conjugate of claim 88, wherein the targeting moiety specifically binds an antigen selected from: OX40L, 4-1BBL, MARCO, DC-SIGN, Dectin1, Dectin2, DEC-205, CLEC5A, CLEC9A, CLEC10A, CLEC12A, CD1A, CD16A, CD32A, CD32B, CD36, CD40, CD47, CD64, CD204, CD206, HVEM, PDL1, mannose scavenger receptor1, and BDCA2.

90. The conjugate of claim 86 or 87, wherein the targeting moiety specifically binds a cell surface antigen on an immune cell that is not an antigen presenting cell.

91. The conjugate of any one of claims 1 to 90, wherein the targeting moiety binds the target (e.g., cell surface antigen) of interest with an equilibrium dissociation constant (Kd) in a range of 0.5 × 10⁻¹⁰ to 10 × 10⁻⁶ as determined using BIACORE® analysis.

92. A method for modulating a nucleic acid or protein level in a cell, said method comprising contacting the cell with a therapeutically effective amount of the conjugate of any one of claims 1-91, wherein the oligonucleotide-HES complex comprises:
(a) a Targeting moiety that binds to a cell surface antigen on the cell or a nearby cell; and
(b) an oligonucleotide that specifically hybridizes to the nucleic acid and modulates the level of the nucleic acid and/or protein encoded or regulated by the nucleic acid.

93. The method of claim 92 wherein the targeting moiety of the T-Oligo-HES conjugate specifically binds a cell surface antigen on the contacted cell and the cell expresses a cell surface protease that cleaves a cleavable linker of the T-Oligo-HES conjugate to release an Oligo-HES complex.

94. The method of claim 92 or 93, wherein the modulated nucleic acid or protein is in a diseased cell, an infected cell, an infectious agent, or an immune cell.

95. The method of claim 94, wherein the modulated nucleic acid or protein is in a diseased cell.

96. The method of claim 95, wherein the modulated nucleic acid or protein is in a cancer cell.

97. The method of claim 96, wherein the cancer cell is a hematologic cancer cell or a solid tumor cancer cell.

98. The method of claim 97, wherein the cancer cell is a leukemic cell, lymphoma cell, pancreatic cancer cell, breast cancer cell, melanoma cell, lung cancer cell, head and neck cancer cell, ovarian cancer cell, bladder cancer cell, a colorectal cancer cell, a prostate cancer cell, a brain cancer cell including a glioblastoma cell, a sarcoma cell, or any carcinoma cell.

99. The method of claims 94, wherein the modulated nucleic acid or protein is in an infected cell or an infectious agent.

100. The method of claim 99, wherein the modulated nucleic acid or protein is in an infectious agent.

101. The method of claim 99 or 100, wherein the cell is infected with: HIV, HTLV-1, Zika Virus, Dengue Virus, Influenza Virus, Ebola Virus, Marburg Virus, Crimean Congo Hemorrhagic Fever Virus, Lassa Fever Virus, Variola Virus, SARS Virus, Rift Valley Fever Virus, TB, Anthrax, Botulism, Tularemia, Plague, Brucellosis, glanders, Mellioidosis, Q fever, or an Alpha virus such as Chikungunya virus, Sindbis virus, Semliki Forest virus, the western, eastern and Venezuelan equine encephalitis viruses, the Ross River virus, COVID, or Influenza.

102. The method of claim 94, wherein the modulated nucleic acid or protein is in an immune cell.

103. The method of 102, wherein the immune cell is a cell of lymphoid of myeloid origin such as a T cell, a B cell, an NK cell, an NKT cell, or a dendritic cell.

104. The method of any one of claims 92 to 103, wherein the therapeutic oligonucleotide is selected from: siRNA, shRNA, miRNA, antagmir, a Dicer substrate, and an antisense.

105. The method of any one of claims 92 to 104, wherein the conjugate comprises a cleavable linker containing an amino acid sequence that is a substrate for at least one protease.

106. The method of claim 105, wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

107. The method of any one of claims 92 to 106, wherein the targeting moiety of the conjugate is an antibody, an antigen binding portion of an antibody (e.g., a Fab, and a scFv), or a single-domain antibody.

108. A method for modulating a nucleic acid or protein level in a subject, said method comprising administering a therapeutically effective amount of the conjugate of any one of claims 1 to 91 to a subject in need thereof, and wherein the conjugate comprises:
(a) a targeting moiety that binds to a cell surface antigen on or near a cell in which the nucleic acid or protein is to be modulated; and
(b) an oligonucleotide that specifically hybridizes to the nucleic acid and modulates the level of the nucleic acid and/or protein encoded or regulated by the nucleic acid.

109. The method of claim 108, wherein the nucleic acid or protein is characterized by
(a) overexpression or underexpression of the nucleic acid in the subject, or
(b) overexpression or underexpression of the protein encoded by the nucleic acid in the subj ect.

110. The method of claim 108 or 109, wherein the modulated nucleic acid or protein is in a diseased cell, an infected cell, an infectious agent, or an immune cell.

111. The method of claim 108, wherein the modulated nucleic acid or protein is in a diseased cell.

112. The method of claim 110, wherein the modulated nucleic acid or protein is in a cancer cell.

113. The method of claim 112, wherein the cancer cell is a hematologic cancer cell or a solid tumor cancer cell.

114. The method of claim 113, wherein the cancer cell is a leukemia cell, lymphoma cell, pancreatic cancer cell, breast cancer cell, melanoma cell, lung cancer cell, head and neck cancer cell, ovarian cancer cell, bladder cancer cell, a colorectal cancer cell, a prostate cancer cell, a brain cancer cell including glioblastoma cell, a sarcoma cell, or any carcinoma cell.

115. The method of claim 110, wherein the modulated nucleic acid or protein is in an infected cell or an infectious agent.

116. The method of claim 115, where the modulated nucleic acid or protein is in an infectious agent.

117. The method of claim 115, wherein the infected cell is an immune cell of lymphoid or myeloid origin.

118. The method of claim 110, wherein the modulated nucleic acid or protein is in an immune cell.

119. The method of claim 118, wherein the immune cell is a lymphoid or a myeloid cell such as, a T cell, a B cell, an NK cell, an NKT cell, or a dendritic cell.

120. The method of any one of claims 108 to 119, wherein the therapeutic oligonucleotide is selected from: a siRNA, a shRNA, a miRNA, an antagmir, a Dicer substrate, and an antisense.

121. The method of any one of claims 108 to 120, wherein the conjugate comprises a cleavable linker containing an amino acid sequence that is a substrate for at least one protease.

122. The method of claim 121, wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

123. The method of any one of claims 108 to 122, wherein the conjugate targeting moiety is an antibody, an antigen binding portion of an antibody (e.g., a Fab, and a scFv), or a single-domain antibody.

124. A method for treating a disease or disorder in a subject, said method comprising administering to a subject in need thereof, a therapeutically effective amount of the conjugate of any one of claims 1 to 91, wherein the oligonucleotide specifically hybridizes to a nucleic acid sequence in vivo and modulates the level of a protein encoded or regulated by the nucleic acid; and wherein the disease or disorder is characterized by:
(a) overexpression or underexpression of the nucleic acid in the subject, or
(b) overexpression or underexpression of the protein encoded by the nucleic acid in the subj ect.

125. The method of claim 124, wherein the conjugate comprises a targeting moiety that binds to a cell surface antigen on or near a cell in which the nucleic acid or protein is to be modulated.

126. The method of claim 124 or 125, wherein the disease or disorder is a proliferative disease or disorder such as cancer, a disease or disorder of the immune system, an inflammatory disease or disorder, an infectious disease, a neurological disease or disorder, a disease or disorder of the cardiovascular system, a metabolic disease or disorder, a disease or disorder of the skeletal system, or a disease or disorder of the skin or eyes.

127. The method of any one of claims 124 to 126, wherein the disease or disorder is cancer, an inflammatory disease or disorder, or a disease or disorder of the immune system, an infectious disease, or a neurological or disease or disorder such as a neurodegenerative disease or disorder.

128. The method of any one of claims 124 to 127, wherein the disease or disorder is cancer.

129. The method of claim 128, wherein the cancer is a hematologic cancer or a solid tumor cancer.

130. The method of claim 129, wherein the cancer is leukemia, pancreatic cancer, breast cancer, melanoma, lung cancer, head and neck cancer, ovarian cancer, bladder cancer, colon cancer, kidney cancer, liver cancer, prostate cancer, bone cancer, or brain cancer including a glioblastoma; or any lymphoma, myeloma, blastoma, sarcoma, leukemia or carcinoma.

131. The method of claim 124, wherein the disease or disorder is an inflammatory disease or disorder, a disease or disorder of the immune system, or an infectious disease.

132. The method of claim 131, wherein the disease is an inflammatory disease or disorder, or an autoimmune disease or disorder (e.g., rheumatoid arthritis).

133. The method of claim 132, wherein the disease or disorder is inflammation.

134. The method of claim 133, wherein the disease or disorder is an infectious disease.

135. The method of claim 134, wherein the infectious disease is HIV, HTLV-1, Zika, Dengue, Influenza, Ebola, Marburg, Crimean Congo Hemorrhagic Fever, Lassa Fever Virus, Variola, SARS, Rift Valley Fever, TB, Anthrax, Botulism, Tularemia, Plague, Brucellosis, glanders, Mellioidosis, Q fever, or infected with an Alpha virus such as Chikungunya virus, Sindbis virus, Semliki Forest virus, the western, eastern and Venezuelan equine encephalitis viruses, the Ross River virus, or COVID.

136. The method of claim 126, wherein the disease or disorder is a neurological disease or disorder.

137. The method of claim 136, wherein the neurological disease or disorder is a neurodegenerative disease such as familial and sporadic amyotrophic lateral sclerosis (FALS and ALS, respectively), familial and sporadic Parkinson’s disease, Huntington’s disease (Huntington’s chorea), familial and sporadic Alzheimer’s disease, Spinal Muscular Atrophy (SMA), multiple sclerosis, diffuse cerebral cortical atrophy, dementia, or Pick disease.

138. The method of any one of claims 124 to 137, wherein the therapeutic oligonucleotide of the conjugate is selected from: a siRNA, a shRNA, a miRNA, an antagmir, a Dicer substrate, and an antisense.

139. The method of any one of claims 124 to 138, wherein the conjugate comprises a cleavable linker containing an amino acid sequence that is a substrate for at least one protease.

140. The method of claim 139, wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

141. The method of any one of claims 124 to 140, wherein the targeting moiety of the conjugate is an antibody, an antigen binding portion of an antibody (e.g., a Fab, and a scFv), or a single-domain antibody.

142. The method of any one of claims 124 to 141, wherein the targeting moiety of the conjugate is an antibody or antigen binding fragment of an antibody.

143. The method of claim 142, wherein the targeting moiety is an antibody or nanaobody.

144. The method of claim 143, wherein the antibody is an IgG1, IgG2, or IgG4 antibody.

145. The method of claim 143 or 144, wherein the antibody is a therapeutic antibody.

146. The method of any one of claims 124 to 145, wherein the conjugate is administered simultaneously, sequentially or separately with one or more other therapeutic drugs.

147. A method of treating cancer in a subject, said method comprising administering a therapeutically effective amount of the conjugate of any one of claims 1 to 91 to a subject in need thereof, wherein the oligonucleotide specifically hybridizes to a nucleic acid in the cancer cell or tissue and modulates the level of the nucleic acid and/or protein encoded or regulated by the nucleic acid, and wherein the nucleic acid or protein is characterized by:
(a) overexpression or underexpression of the nucleic acid in the cancer cell, tissue, and/or subj ect, or
(b) overexpression or underexpression of the protein encoded by the nucleic acid in the cancer cell, tissue, and/or subject.

148. The method of claim 147, wherein the targeting moiety of the conjugate specifically binds to a cell surface antigen on or near the cancer cell.

149. The method of claim 147 or 148, wherein the targeting moiety of the conjugate specifically binds to a cell surface antigen on the cancer cell.

150. The method of any one of claims 147 to 149, wherein the targeting moiety of the conjugate specifically binds to a cell surface antigen on a cell near the cancer cell (e.g., a cell in the tumor microenvironment such as a stromal cell, cancer associated fibroblast, immune cell, blood or lymphatic vascular cell, endothelial cell, adipose cell, or neuroendocrine cell).

151. The method of any one of claims 147 to 150, wherein the cancer is a solid tumor.

152. The method of any one of claims 147 to 151, wherein the cancer is leukemia, pancreatic cancer, breast cancer, melanoma, lung cancer, head and neck cancer, ovarian cancer, bladder cancer, colon cancer, kidney cancer, liver cancer, prostate cancer, bone cancer, or brain cancer including a glioblastoma; or any lymphoma, myeloma, blastoma, sarcoma, leukemia or carcinoma.

153. The method of claim 152, wherein the cancer is a hematologic cancer.

154. The method of claim 153, wherein the hematologic cancer is a leukemia or lymphoma.

155. The method of any one of claims 147 to 154, wherein the targeting moiety of the conjugate specifically binds a cell surface antigen selected from: CD5, CD19, CD20, CD25, CD37, CD30, CD33, CD45, CD204, CD206, CD301, CAMPATH-1, HLD-DR, carcinoembryonic antigen (CEA), TAG-72, EpCAM, MUC1, MUC15, folate-binding protein, A33, G250, prostate-specific membrane antigen (PSMA), ferritin, GD2, GD3, GM2, Ley, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, HIV GP120, HIV GP160, EGFRvIII, HER2/neu, MAGE A3, P53 nonmutant, NY-ESO-1, MelanA/MART1, Ras mutant, gp100, P53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, MAGE-A3, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, CMET, HER3, CA6, NAPI2B, TROP2, CLDN18.2, fibroblast activation protein (FAP), RON, LY6E, FRA, DLL3, PTK7, LIV1, ROR1, Fos-related antigen 1, VEGFR, endoglin, PDL, VTCN1, and VISTA.

156. The method of any one of claims 147 to 154, wherein the targeting moiety of the conjugate specifically binds a cell surface antigen selected from: DLL3, fibroblast activation protein α (FAPα), NG2 (Neuroglial Antigen-2), platelet-derived growth factor receptor-β (PDGFR-β), PD1, CD163, KIR, HMGB1, VEGFR3, LYVE1, CD31, CD34, P1GF, and VEGF.

157. The method of any one of claims 147 to 154, wherein the targeting moiety of the conjugate specifically binds a cell surface antigen selected from: HER2, EGFR, CMET, HER3, MUC1, MUC16, EPCAM, MSLN, CA6, NAPI2B, TROP2, CEA, CLDN18.2, EGFRvIII, FAP, EphA2, RON, LY6E, FRA, PSMA, DLL3, PTK7, LIV1, ROR1, MAGE-A3, NY-ESO-1, Endoglin, CD204, CD206, CD301, VTCN1, VISTA, GLP-3, CLDN6, CLDN16, UPK1B, STRA6, TMPRSS3, TMPRSS4, TMEM238, C1orf186, and LRRC15.

158. The method of any one of claims 147 to 156, wherein the therapeutic oligonucleotide of the conjugate is selected from: a siRNA, a shRNA, a miRNA, an antagmir, a Dicer substrate, and an antisense.

159. The method according to any one of claims 147 to 157, wherein the therapeutic oligonucleotide of the conjugate specifically hybridizes to a nucleic acid selected from: EGFR, HER2/neu, ErbB3, cMet, p56lck, PDGFR, VEGF, VEGFR, FGF, FGFR, ANG1, ANG2, bFGF,TIE2, protein kinase C-alpha (PKC-alpha), p56lck PKA, TGF-beta, IGFIR, P12, MDM2, BRCA, IGF1, HGF, PDGF, IGFBP2, IGF1R, HIF1 alpha, ferritin, transferrin receptor, TMPRSS2, IRE, HSP27, HSP70, HSP90, MITF, clusterin, PARP1C-fos, C-myc, n-myc, C-raf, B-raf, A1, H-raf, Skp2, K-ras, N-ras, H-ras, farensyltransferase, c-Src, Jun, Fos, Bcr-Abl, c-Kit, EphA2, PDGFB, ARF, NOX1, NF1, STAT3, E6/E7, APC, WNT, beta catenin, GSK3b, PI3k, mTOR, Akt, PDK-1, CDK, Mek1, ERK1, AP-1, P53, Rb, Syk, osteopontin, CD44, MEK, MAPK, NF kappa beta, E cadherin, cyclin D, cyclin E, Bcl2, Bax, BXL-XL, BCL-W, MCL1, ER, MDR, telomerase, telomerase reverse transcriptase, a DNA methyltransferase, a histone deacetlyase (e.g., HDAC1 and HDAC2), an integrin, an IAP, an aurora kinase, a metalloprotease (e.g., MMP2, MMP3 and MMP9), a proteasome, or a metallothionein gene.

160. The method according to any one of claims 147 to 157, wherein the therapeutic oligonucleotide of the conjugate specifically hybridizes to a nucleic acid selected from: survivin, HSPB1, EIF4E, PTPN1, RRM2, BCL2, PTEN, Bcr-abl, TLR9, HaRas, Pka-rIA, JNK2, IGF1R, XIAP, TGF-β2, c-myb, PLK1, K-ras, KSP, PKN3, a Ribonucleotide Reductase (e.g., Ribonucleotide Reductase R1 and Ribonucleotide Reductase R2), a RecQ helicase (e.g., WRN, RecQL1, BLM, RecQL4, RecQ5, and RTS), MEM2 and TLR9.

161. The method of any one of claims 147 to 159, wherein the conjugate comprises a cleavable linker containing an amino acid sequence that is a substrate for at least one protease.

162. The method of claim 160, wherein the cleavable linker contains an amino acid sequence that is a substrate for at least one protease selected from: a metalloproteinase (e.g., Meprin, Neprilysin, PSMA, and BMP1); a matrix metalloprotease (e.g., MMP1-3, MMP 7-17, MMP 19, MMP 20, MMP 23, MMP 24, MMP 26, and MMP 27), thrombin, an elastase (e.g., human neutrophil elastase), a cysteine protease (e.g., legumain and cruzipain), a serine protease (e.g., Cathepsin C, and a TTSP such, as DECC1, FAP, Matriptase-2, MT-SPl/Matriptase, and TMPRSS2-4), Urokinase (uPA), an aspartate protease (e.g., BACE and Renin); an aspartic cathepsin (e.g., Cathepsin D), and a threonine protease.

163. The method of any one of claims 147 to 161, wherein the conjugate targeting moiety is an antibody, an antigen binding portion of an antibody (e.g., a Fab, and a scFv), or a single-domain antibody.

164. The method of any one of claims 147 to 162, wherein the targeting moiety of the conjugate is an antibody or antigen binding fragment of an antibody.

165. The method of claim 163, wherein the targeting moiety is an antibody.

166. The method of claim 164, wherein the antibody is an IgG1, IgG2, or IgG4 antibody.

167. The method of claim 164 or 165, wherein the antibody is a therapeutic antibody.

168. The method of claim 166, wherein the antibody is selected from: trastuzumab (HER2/neu), pertuzumab (HER2/neu), panitumumab (EGFR), nimotuzumab (EGFR), zalutumumab (EGFR), cetuximab (EGFR), (HER3), onartuzumab (c-MET), patritumab, clivatuzumab (MUC1), sofituzumab (MUC16), edrecolomab, (EPCAM), adecatumumab (EPCAM), anetumab (MSLN), huDS6 (CA6), lifastuzumab (NAPI2B), sacituzumab (TROP2), PR1A3, humanized PR1A3 (CEA), humanized Ab 2-3 (CEA), IMAB362/claudiximab (Claudin18.2), AMG595 (EGFRvIII), ABT806 (EGFRvIII), sibrotuzumab (FAP), DS-8895a variant 1 (EphA2), DS-8895a variant 2 (EphA2), anti-EphA2 (EphA2), MEDI-547 (EphA2), narnatumab (RON), RG7841 (LY6E), farletuzumab (FRA /folate receptor alpha), mirvetuximab (FRA), J591 variant 1 (PSMA), J591 variant 2 (PSMA), rovalpituzumab (DLL3), PF-06647020 (PTK7), anti-PTK7 (PTK7), ladiratuzumab (LIV1), cirmtuzumab (ROR1), rituximab (CD20), ibritumomab tiuxetan (CD52), alemtuzumab (CD33), Gemtuzumab ozogamicin (CD33), CT-011 (PD1), tositumomab (CD20), ipilimumab (CTLA4), tremelimumab (CP-675,206)(CTLA4), nivolumab (PD1), and pembrolizumab (PD1), durvalumab (PDL1) anti-MAGE-A3, and anti-NY-ESO-1.

169. The method of any one of claims 147 to 167, wherein the conjugate is administered simultaneously, sequentially or separately with one or more other anticancer drugs.

170. A conjugate according to any one of claims 1 to 91 for use in medicine.

171. A conjugate as defined to any one of claims 1 to 91 for use in treatment of a disease or disorder in a subject.

172. The conjugate according to any one of claims 1 to 91 for use in treating a disease or disorder selected from: an infectious disease, cancer, a proliferative disease or disorder, a neurological disease or disorder, and inflammatory disease or disorder, a disease or disorder of the immune system, a disease or disorder of the cardiovascular system, a metabolic disease or disorder, a disease or disorder of the skeletal system, and a disease or disorder of the skin or eyes.

173. A conjugate according to any one of claims 1 to 91 for use in modulating a target nucleic acid or protein subject; treating a disease or disorder characterized by overexpression or underexpression of a nucleic acid in a subject, treating a disease or disorder characterized by overexpression or underexpression of a protein in a subject; treating a disease or disorder characterized by aberrant nucleic acid or protein expression in a subject.


SEQUENCE LISTING (87 sequences)

SEQ ID NO: 1 - DNA, 24 bp, Artificial Sequence
  synthesized complement of Beta-actin
cccggcgatatcatcatccataac

SEQ ID NO: 2 - DNA, 20 bp, Artificial Sequence
  PKC-alpha binding oligonucleotide
gttctcgctggtgagtttca

SEQ ID NO: 3 - DNA, 20 bp, Artificial Sequence
  P53 binding oligonucleotide
ccctgctcccccctggctcc

SEQ ID NO: 4 - DNA, 20 bp, Artificial Sequence
  H-Ras binding oligonucleotide
tccgtcatcgctcctcaggg

SEQ ID NO: 5 - DNA, 20 bp, Artificial Sequence
  Ha-Ras binding oligonucleotide
gggactcctcgctactgcct

SEQ ID NO: 6 - DNA, 20 bp, Artificial Sequence
  c-Raf binding oligonucleotide
tcccgcctgtgacatgcatt

SEQ ID NO: 7 - DNA, 18 bp, Artificial Sequence
  Bcl2 binding oligonucleotide
tctcccagcgtgcgccat

SEQ ID NO: 8 - DNA, 21 bp, Artificial Sequence
  VEGF binding oligonucleotide
tggcttgaagatgtactcgat

SEQ ID NO: 9 - DNA, 20 bp, Artificial Sequence
  Ribonucleotide Reductase R2 binding oligonucleotide
ggctaaatcgctccaccaag

SEQ ID NO: 10 - DNA, 24 bp, Artificial Sequence
  c-myb binding oligonucleotide
tatgctgtgccggggtcttcgggc

SEQ ID NO: 11 - DNA, 26 bp, Artificial Sequence
  Bcr-abl binding oligonucleotide
cgctgaagggcttcttccttattgat

SEQ ID NO: 12 - DNA, 25 bp, Artificial Sequence
  Bcr-abl binding oligonucleotide
cgctgaagggctttgaactgtgctt

SEQ ID NO: 13 - DNA, 18 bp, Artificial Sequence
  Pka-rIA binding oligonucleotide
gcgugcctcctcacuggc

SEQ ID NO: 14 - DNA, 15 bp, Artificial Sequence
  c-Myc binding oligonucleotide
aacgttgaggggcat

SEQ ID NO: 15 - DNA, 20 bp, Artificial Sequence
  JNK2 binding oligonucleotide
gctcagtggacatggatgag

SEQ ID NO: 16 - DNA, 18 bp, Artificial Sequence
  IGF1-R binding oligonucleotide
ggaccctcctccggagcc

SEQ ID NO: 17 - DNA, 20 bp, Artificial Sequence
  PTEN binding oligonucleotide
ctgctagcctctggatttga

SEQ ID NO: 18 - DNA, 22 bp, Artificial Sequence
  TLR9 binding oligonucleotide
tgactgtgaacgttcgagatga

SEQ ID NO: 19 - DNA, 20 bp, Artificial Sequence
  ICAM-1 binding oligonucleotide
gcccaagctggcatccgtca

SEQ ID NO: 20 - DNA, 20 bp, Artificial Sequence
  TNF-alpha binding oligonucleotide
gctgattagagagaggtccc

SEQ ID NO: 21 - DNA, 21 bp, Artificial Sequence
  Adenosine A1 receptor binding oligonucleotide
gatggagggcggcatggcggg

SEQ ID NO: 22 - DNA, 25 bp, Artificial Sequence
  HIV/Gag binding oligonucleotide
ctctcgcacccatctctctccttct

SEQ ID NO: 23 - DNA, 21 bp, Artificial Sequence
  CMV IE2 binding oligonucleotide
gcgtttgctcttcttcttgcg

SEQ ID NO: 24 - DNA, 20 bp, Artificial Sequence
  HCV binding oligonucleotide
gtgctcatggtgcacggtct

SEQ ID NO: 25 - DNA, 24 bp, Artificial Sequence
  CMV binding oligonucleotide
tcgtcgttttgtcgttttgtcgtt

SEQ ID NO: 26 - RNA, 28 nt, Artificial Sequence
  RSV RSV binding oligonucleotide
ggcucuuagcaaagucaaguugaaugau

SEQ ID NO: 27 - RNA, 30 nt, Artificial Sequence
  RSV binding oligonucleotide
aucauucaacuugacuuugcuaagagccau

SEQ ID NO: 28 - RNA, 21 nt, Artificial Sequence
  RSV RSV binding oligonucleotide
  misc_feature LOCATION: (20)..(21): 2'-deoxythymidines
cgauaauauaacugcaagann

SEQ ID NO: 29 - RNA, 21 nt, Artificial Sequence
  RSV binding oligonucleotide
  misc_feature LOCATION: (1)..(2): 2'-deoxythymidines
nngcuauuauauugacguucu

SEQ ID NO: 30 - RNA, 21 nt, Artificial Sequence
  RSV binding oligonucleotide
  misc_feature LOCATION: (20)..(21): 2'-deoxythymidines
ugcuguaacagaauugcagnn

SEQ ID NO: 31 - RNA, 21 nt, Artificial Sequence
  RSV binding oligonucleotide
  misc_features LOCATION: (20)..(21): 2'-deoxythymidines
  misc_feature LOCATION: (20)..(21): n is a, c, g, or u
cugcaauucuguuacagcann

SEQ ID NO: 32 - DNA, 22 bp, Artificial Sequence
  VP35 binding oligonucleotide
  misc_feature LOCATION: (1)..(1): n is Amino dT c6
  misc_feature LOCATION: (22)..(22): n is Amino dT c6
ncctgccctttgttctagttgn

SEQ ID NO: 33 - RNA, 21 nt, Artificial Sequence
  Ebola VP35 binding oligonucleotide
gcgacaucuucugugauauug

SEQ ID NO: 34 - RNA, 21 nt, Artificial Sequence
  Ebola VP35 binding oligonucleotide
auaucacagaagaugucgcuu

SEQ ID NO: 35 - RNA, 19 nt, Artificial Sequence
  Ebola VP35 binding oligonucleotide
cauuacgagucuugagaau

SEQ ID NO: 36 - RNA, 19 nt, Artificial Sequence
  Ebola VP35 binding oligonucleotide
ucucaagacucguaaugcg

SEQ ID NO: 37 - RNA, 21 nt, Artificial Sequence
  Ebola VP35 binding oligonucleotide
gcaacucauuggacaucauuc

SEQ ID NO: 38 - RNA, 21 nt, Artificial Sequence
  Ebola VP35 binding oligonucleotide
augauguccaaugaguugcua

SEQ ID NO: 39 - RNA, 19 nt, Artificial Sequence
  Ebola VP35 binding oligonucleotide
ugaugaagauuaagaaaaa

SEQ ID NO: 40 - RNA, 19 nt, Artificial Sequence
  Ebola VP35 binding oligonucleotide
uuucuuaaucuucaucacu

SEQ ID NO: 41 - RNA, 19 nt, Artificial Sequence
  Ebola VP35 binding oligonucleotide
gugcugagaugguugcaaa

SEQ ID NO: 42 - RNA, 19 nt, Artificial Sequence
  Ebola VP35 binding oligonucleotide
ugcaaccaucucagcacaa

SEQ ID NO: 43 - RNA, 19 nt, Artificial Sequence
  Ebola VP35 binding oligonucleotide
gcuaaugaccggaagaauu

SEQ ID NO: 44 - RNA, 19 nt, Artificial Sequence
  Ebola VP35 binding oligonucleotide
uucuuccggucauuagcug

SEQ ID NO: 45 - RNA, 19 nt, Artificial Sequence
  Ebola VP35 binding oligonucleotide
ccaauuaguacaagugauu

SEQ ID NO: 46 - RNA, 19 nt, Artificial Sequence
  Ebola VP35 binding oligonucleotide
ucacuuguacuaauuggug

SEQ ID NO: 47 - DNA, 22 bp, Artificial Sequence
  NP binding oligonucleotide
  misc_feature LOCATION: (1)..(1): n is Amino dT c6
  misc_feature LOCATION: (22)..(22): n is Amino dT c6
ngagaatccatactcggaattn

SEQ ID NO: 48 - DNA, 22 bp, Artificial Sequence
  NP binding oligonucleotide
  misc_feature LOCATION: (1)..(1): n is Amino dT c6
  misc_feature LOCATION: (22)..(22): n is Amino dT c6
ngacgagaatccatactcggan

SEQ ID NO: 49 - DNA, 21 bp, Artificial Sequence
  NP binding oligonucleotide
  misc_feature LOCATION: (1)..(1): n is Amino dT c6
  misc_feature LOCATION: (21)..(21): n is Amino dT c6
ngcatgtacttgaatttgccn

SEQ ID NO: 50 - RNA, 21 nt, Artificial Sequence
  Ebola NP binding oligonucleotide
  misc_feature LOCATION: (20)..(21): 2'-deoxythymidines
ggcaaauucaaguacaugcnn

SEQ ID NO: 51 - RNA, 21 nt, Artificial Sequence
  Ebola NP binding oligonucleotide
gcauguacuugaauuugccuu

SEQ ID NO: 52 - RNA, 21 nt, Artificial Sequence
  Ebola NP binding oligonucleotide
gcauggagaguaugcuccuuu

SEQ ID NO: 53 - RNA, 21 nt, Artificial Sequence
  Ebola NP binding oligonucleotide
aggagcauacucuccaugcuu

SEQ ID NO: 54 - DNA, 20 bp, Artificial Sequence
  Ebola NP binding oligonucleotide
atggtgattttccgtttgat

SEQ ID NO: 55 - DNA, 19 bp, Artificial Sequence
  Ebola NP binding oligonucleotide
tcaaacggaaaatcaccat

SEQ ID NO: 56 - DNA, 19 bp, Artificial Sequence
  Ebola NP binding oligonucleotide
gagaagcaactccaacaat

SEQ ID NO: 57 - RNA, 17 nt, Artificial Sequence
  Ebola NP binding oligonucleotide
uguuggaguugcuucuc

SEQ ID NO: 58 - DNA, 22 bp, Artificial Sequence
  RNA polymerase L binding oligonucleotide
  misc_feature LOCATION: (1)..(1): n is Amino dT c6
  misc_feature LOCATION: (22)..(22): n is Amino dT c6
ntgggtatgttgtgtagccatn

SEQ ID NO: 59 - RNA, 21 nt, Artificial Sequence
  Ebola L Polymerase binding oligonucleotide
guacgaagcuguauauaaauu

SEQ ID NO: 60 - RNA, 21 nt, Artificial Sequence
  Ebola L Polymerase binding oligonucleotide
uuuauauacagcuucguacuu

SEQ ID NO: 61 - DNA, 21 bp, Artificial Sequence
  VP24 binding oligonucleotide
  misc_feature LOCATION: (1)..(1): n is Amino dT c6
  misc_feature LOCATION: (21)..(21): n is Amino dT c6
ngccatggttttttctcaggn

SEQ ID NO: 62 - RNA, 21 nt, Artificial Sequence
  Ebola VP24 binding oligonucleotide
gcugauugaccagucuuugau

SEQ ID NO: 63 - RNA, 21 nt, Artificial Sequence
  Ebola VP24 binding oligonucleotide
caaagacuggucaaucagcug

SEQ ID NO: 64 - RNA, 21 nt, Artificial Sequence
  Ebola VP24 binding oligonucleotide
acggauuguugagcaguauug

SEQ ID NO: 65 - RNA, 21 nt, Artificial Sequence
  Ebola VP24 binding oligonucleotide
auacugcucaacaauccguug

SEQ ID NO: 66 - RNA, 21 nt, Artificial Sequence
  Ebola VP24 binding oligonucleotide
uccucgacacgaaugcaaagu

SEQ ID NO: 67 - RNA, 21 nt, Artificial Sequence
  Ebola VP24 binding oligonucleotide
uuugcauucgugucgaggauc

SEQ ID NO: 68 - RNA, 19 nt, Artificial Sequence
  Influenza NP binding oligonucleotide
ggaucuuauuucuucggag

SEQ ID NO: 69 - RNA, 19 nt, Artificial Sequence
  Influenza NP binding oligonucleotide
cuccgaagaaauaagaucc

SEQ ID NO: 70 - RNA, 19 nt, Artificial Sequence
  Influenza PA binding oligonucleotide
gcaauugaggagugccuga

SEQ ID NO: 71 - RNA, 19 nt, Artificial Sequence
  Influenza PA binding oligonucleotide
ucagcgacuccucaauugc

SEQ ID NO: 72 - RNA, 19 nt, Artificial Sequence
  Influenza PB1 binding oligonucleotide
gaucuguuccaccauugaa

SEQ ID NO: 73 - RNA, 19 nt, Artificial Sequence
  Influenza PB1 binding oligonucleotide
uucaaugguggaacagauc

SEQ ID NO: 74 - RNA, 19 nt, Artificial Sequence
  Influenza M2 binding oligonucleotide
acagcagaaugcuguggau

SEQ ID NO: 75 - RNA, 19 nt, Artificial Sequence
  Influenza M2 binding oligonucleotide
auccacagcauucugcugu

SEQ ID NO: 76 - DNA, 20 bp, Artificial Sequence
  PTP1VB binding oligonucleotide
gctccttccactgatcctgc

SEQ ID NO: 77 - protein, 5 aa, Homo sapiens
PLGIA

SEQ ID NO: 78 - protein, 23 aa, Artificial Sequence
  PLGIA Cleavage site peptide
  MISC_FEATURE LOCATION: (2)..(2): Xaa is 6-aminoahexanoic acid residue (C5)
  MISC_FEATURE LOCATION: (6)..(6): Xaa is 6-aminoahexanoic acid residue (C5)
  MISC_FEATURE LOCATION: (16)..(16): Xaa is 6-aminoahexanoic acid residue (C5)
  MISC_FEATURE LOCATION: (19)..(19): Xaa is 6-aminoahexanoic acid residue (C5)
EXKDPXGPLGIAGIGXPKXGCGY

SEQ ID NO: 79 - DNA, 32 bp, Artificial Sequence
  Kras binding oligonucleotide
taggaaaagctattaggagtctttatagtata

SEQ ID NO: 80 - DNA, 30 bp, Artificial Sequence
  Arm oligonucleotide
cucuccttctagcctccgctagtcaaaauu

SEQ ID NO: 81 - DNA, 28 bp, Artificial Sequence
  Arm oligonucleotide
attttgactagcggaggctagaaggaga

SEQ ID NO: 82 - DNA, 20 bp, Artificial Sequence
  BCL2 binding oligonucleotide
aaggcatcccagcctccgtt

SEQ ID NO: 83 - DNA, 30 bp, Artificial Sequence
  BCL2 binding oligonucleotide
ccacaaaggcatcccagcctccgttatcct

SEQ ID NO: 84 - DNA, 30 bp, Artificial Sequence
  KRAS oligonucleotide
auacuaaaucauuugaagauauucaccaut

SEQ ID NO: 85 - DNA, 30 bp, Artificial Sequence
  KRAS oligonucleotide
tauggugaauaucuucaaaugauuuaguau

SEQ ID NO: 86 - DNA, 31 bp, Artificial Sequence
  KRAS oligonucleotide
aguacagugcaaugagggaccaguacaugat

SEQ ID NO: 87 - DNA, 31 bp, Artificial Sequence
  KRAS oligonucleotide
tucauguacuggucccucauugcacuguacu